(19) United States
(12) Patent Application Publication
(10) Pub. No.: US 2026/0055363 A1
(43) Pub. Date: Feb. 26, 2026

(54) A MICROCAPSULE AND METHODS OF MAKING AND USING SAME

(71) Applicant: VILNIUS UNIVERSITY, Vilnius (LT)

(72) Inventors: Linas MAZUTIS, Vilnius (LT); Greta LEONAVICIENE, Vilnius (LT)

(21) Appl. No.: 18/715,650

(22) PCT Filed: Dec. 1, 2022
(86) PCT No.: PCT/EP2022/084071

§ 371 Date: May 31, 2024

Related U.S. Application Data
(60) Provisional application No. 63/284,657, filed on Dec. 1, 2021.

(51) Int. Cl. C12N 5/00; A61K 9/50; A61K 47/10; A61K 47/32; C12M 1/12; C12N 1/04; C12N 15/88

(52) U.S. Cl. CPC C12N 5/0012; A61K 9/5036; A61K 9/5057; A61K 47/10; A61K 47/32; C12M 25/16; C12N 1/04; C12N 15/88; C12N 2513/00; C12N 2533/40; C12N 2533/54

(57) ABSTRACT

The present disclosure provides in one aspect a microcapsule comprising: (a) a core comprising a polyhydroxy compound and/or an antichaotropic agent; and (b) a semi-permeable shell surrounding the core; wherein the semi-permeable shell comprises a gel formed from a polyampholyte, wherein the polyampholyte in the gel is covalently cross-linked. The present disclosure provides further aspects relating to a method of making a microcapsule, methods of using the microcapsule and kits for making a microcapsule.

### CROSS-REFERENCE TO RELATED APPLICATIONS

This application is the U.S. national phase of International Application No. PCT/EP2022/084071 filed Dec. 1, 2022 which designated the U.S. and claims priority to U.S. 63/284,657 filed Dec. 1, 2021, the entire contents of each of which are hereby incorporated by reference.

### INCORPORATION BY REFERENCE

The text file named V8392-seq1-000001, created on Jan. 5, 2023, and sized 6,284 bytes, which contains sequence ID listings, is herein expressly incorporated by reference.

### FIELD OF THE INVENTION

The present invention relates to a microcapsule comprising a semi-permeable shell. In particular, the microcapsule can comprise at least one biological entity, such as a cell, a microorganism, a bacterium, a virus, or a nucleic acid, or a part of any of the foregoing. The microcapsule can be utilized in methods of culturing, expanding, analyzing and/or storing the at least one biological entity. The microcapsules can also be utilized in vivo, for the delivery of the at least one biological entity, in a method of treatment, in particular where the method of treatment is cell therapy. The present invention also concerns a method for making the microcapsule, kits suitable for performing the method, and microcapsules produced by such a method.

### BACKGROUND OF INVENTION

Three-dimensional (3D) cell assemblies (structures) such as spheroids, organoids, tumoroids, tissues and other cell masses have important applications in biomedical and biological research. For example, 3D organoids can serve as useful models of complex diseases [1], drug screening [2], developmental biology, tissue engineering. Spheroids and tumoroids provide an important in vitro 3D model for studying cell death and response of tumors to radiotherapy and chemotherapy as well as cell development [3, 4]. Although most of the conventional cell culture methods rely on 2D cultures, where the cells are seeded and harvested in a cell culture dish, numerous efforts have been dedicated to develop tools and methods for in vitro 3D cell cultures (spheroids, organoids, tumoroids, etc). [5]. For example, 3D cell structures (e.g., spheroids) have been successfully generated using cell culture [6], microtiter plates [7], hanging drop [8], and even magnetic levitation methods [9]. However, these methods are cumbersome to implement, are low-throughput, do not comply with perfusion protocols and tend to generate 3D cell assemblies with nonuniform sizes. The use of microfluidics technology may circumvent some of these limitations [10]. Encapsulating cells into liquid (water-in-oil) droplets provide a high-throughput tool to generate uniform spheroids, yet it enables cell culture for just a few days [11]. During cell culture, a finite amount of nutrients available in the water-in-oil droplet gets depleted and as a result cell physiological functions may be significantly altered. Moreover, the soft interphase of water-in-oil droplets is unsuitable for culturing adherent cells, which rely on a solid substrate for maintaining viability and cellular functions.

The cell encapsulation using different materials have a long history of research [12, 13]. Encapsulating cells into hydrogel beads (e.g. composed of alginate, agarose, collagen, etc.) creates a 3D environment, which provides a structural support for encapsulated cell growth and function as well as formation of complex 3D assemblies such as spheroids [14-17]. In addition, cells within the hydrogel bead can be maintained in culture for a long period of time, and may be supplied with fresh nutrients that can diffuse into the hydrogel beads and may be consumed (e.g., glucose, amino acids) by the encapsulated cells. Indeed, the use of hydrogel beads have found many useful applications over the last few years (see a review by [18]). However, the use of hydrogel beads is not without limitations. First, since encapsulated cells get entangled in the hydrogel mesh, it is challenging to achieve desirable 3D cell assemblies since the hydrogel polymer may physically obstruct the encapsulated cells from forming required cell-cell interactions. For example, Yu et al., showed that cell entanglement in hydrogels may reduce the proliferation of encapsulated cells due to the increased hydrogel rigidity [19]. Siltanen et al., showed that while hydrogel beads supported the culture and differentiation of the encapsulated mouse embryonic stem cells [20], yet the encapsulated primary hepatocytes or human embryonic stem cells did not form the 3D cell assemblies such as spheroids, thus implying that hydrogel mesh of the beads impose physiological constrains on encapsulated cells. Therefore, for some applications it may be advantageous to have encapsulated cells in a liquid environment so that the cells could interact with each other without physical, electrostatic or other type of obstruction by the hydrogel mesh. Second, the spatial position of encapsulated cells inside the hydrogel beads is difficult to control. Once the cells are encapsulated into hydrogel beads, the said cells will randomly distribute in the entire volume of the hydrogel beads and given the large surface to volume ratio of a hydrogel bead, a significant fraction of encapsulated cells will end-up at the interface, or at the proximity to the interface [21]. As a result of this partition the cells may escape hydrogel beads during prolonged incubations such as culture, harvesting or testing. The cells may also remain adhered to the outer side of the hydrogel bead. In either case uncontrolled cell partition is undesirable for many biological testing and assays. Third, in many cases the hydrogel beads are reversibly cross-linked, for example, by exploiting the ionic interactions (calcium alginate) or making use of thermo-sensitive properties of polymers (agarose). The reversible cross-linking facilitates encapsulate cell release at a desirable step in the protocol, yet the use of such hydrogels becomes restricted to the conditions that sustain the cross-linking of polymer chains. For instance, gels based on calcium alginate are sensitive to pH changes, ions (e.g., phosphate) and chelating agents (e.g., citric acid), while thermosensitive gels such as agarose may partially melt at physiological temperatures (˜37° C.).

Microcapsules, having a liquid core and a hydrogel shell [22, 23]; as well as composite microcapsules having a core-shell structure where an inner core composed of one hydrogel type is surrounded by a shell composed of another hydrogel type [21], have found numerous applications in cell biology, biotechnology and biomedicine as reviewed by Jo and Lee et al., [24] and Huang et al., [25]. One of the main advantages provided by microcapsules is that the encapsulated cells are contained within the inner core of the microcapsule and cannot transverse the outer shell and escape compartmentalization. Therefore, cell encapsulation efficiency of microcapsules is higher than for hydrogel beads [21]. Second, the solidified shell acts as a sieve enabling passive nutrient exchange between the interior and exterior environments, and by doing so ensures cell survival, growth and metabolic functions. By controlling the porosity of the shell, the permeability to molecular compounds can be precisely controlled. Third, because encapsulated cells are suspended in a liquid core, the establishment of cell-cell interactions and formation of 3D cell assemblies may be unhindered as compared to entangled cells cultured within the hydrogel mesh. Fourth, by controlling the diameter of the microcapsule, and more specifically the diameter of the inner core, the size of the 3D cell assemblies (e.g. spheroids, tumoroids) can be precisely tuned.

The most common are core-shell microcapsules composed of a liquid core and a solidified alginate shell [22, 23, 26]. Such microcapsules are typically produced by first encapsulating the cell suspension into liquid droplets surrounded by alginate liquid shell and then solidifying the alginate shell upon reacting with cross-linking ions such as calcium or barium [23]. Unfortunately, such capsules are unstable in the presence of competing ions, dissolve in the presence of acids (e.g., citric acid), start to disintegrate at pH<7.0, and may lose integrity over longer periods of time (>14 days) in cell culture. In addition, it is known that calcium acts as a secondary messenger in stimulus-response reactions of cells, and therefore presence of high concentration of this secondary messenger may alter the physiological state, growth and response of the encapsulated cells. Over the last decade multiple groups have shown generation of cell-loaded capsules having a liquid core and a solid shell, where the shell is composed of polysaccharides [21-23, 26, 27] or PEG hydrogel [28], as reviewed elsewhere [18, 24, 25]. Despite using different methods to generate the capsules, the vast majority of cell loaded microcapsules reported to-date comprise the polysaccharide hydrogel shells. Such capsules lack the mechanical and chemical stability that may be required for long-term culture and assaying of the encapsulated cells. For example, the use of citric acid, phosphate ions, salts and other reagents may compromise the capsule's shell. Furthermore, the polysaccharide shells have generally weak cell adhesion properties [29]. Microfluidics technology has previously been employed for the generation of microcapsules. For example, using microfluidics device the water-in-oil droplets comprising aqueous two-phase system can be generated and cells encapsulated [30]. If the right conditions and composition are found, the liquid-liquid phase separation within the water-in-oil droplets may result in a liquid core and a liquid shell. By converting the liquid shell to solidified state the microcapsule can be formed [23, 31-33]. However, the decades of research and efforts have been so far unsuccessful in generating the microcapsules comprising encapsulated cells, and having suitable properties to withstand a broad range of experimental conditions.

The applicant has previously described the method for producing the semi-permeable capsules composed of the polyethylene glycol (PEG) hydrogel shell and polysaccharide hydrogel core (U.S. patent application Ser. No. 16/934,045; Published as US 2020/0400538) and [31]. These disclosures include examples in which the microcapsule is composed of dextran-rich solution that forms microcapsule's core, and modified PEG polymer (e.g., polyethylene glycol diacrylate (PEGDA) polymer) that forms a solid shell. Since PEG-based polymers, including PEGDA, PEGDMA, PEGMA etc. are partly soluble in dextran phase, upon polymerization the microcapsule's core forms a covalently crosslinked hydrogel mesh, and cannot be converted back to a liquid form. Therefore, in such examples the said microcapsule can be considered as a core-shell microparticle composed of two hydrogel layers; hydrogel (weak-hydrogel) constituting the inner core of a microcapsule and rich in dextran, and hydrogel (stiff-hydrogel) constituting the outer shell of a microcapsule composed of polymerized acrylate-functionalized polyethylene glycol. In addition, the PEG polymer constituting the semi-permeable shell of a microcapsule is biologically and biochemically inert, thus encapsulated cells cannot adhere to the said shell. Further, as stated in the article [31], “ . . . the generation of larger size concentric capsules becomes increasingly difficult to achieve.”, and therefore the utility of the microcapsules may be limited in some embodiments.

### SUMMARY OF THE INVENTION

Accordingly, in a first aspect the present invention provides a microcapsule comprising:
(a) a core comprising a polyhydroxy compound and/or an antichaotropic agent; and
(b) a semi-permeable shell surrounding the core;
   wherein the semi-permeable shell comprises a gel formed from a polyampholyte and/or a polyelectrolyte, wherein the polyampholyte in the gel is covalently cross-linked.

Optionally, the microcapsule may comprise at least one biological entity, such as a cell, a microorganism, a bacteria, a virus, or a nucleic acid, or a part of any of the foregoing. The at least one biological entity may be comprised in the core of the microcapsule or attached to the outer surface of the semi-permeable shell. Where when the at least one biological entity is comprised in the core it may be at the inner surface of the semi-permeable shell or attached to the inner surface of the semi-permeable shell.

In a second aspect, the present invention provides a plurality of the microcapsules of the first aspect.

In a third aspect, the present invention provides a composition comprising a microcapsule according of the first aspect, or a plurality of microcapsule according to the second aspect, in a carrier oil, or an aqueous solution.

In a fourth aspect, the present invention provides a method of producing a microcapsule according to the first aspect, the method comprising:
(a) forming a droplet from a first solute and a second solute, wherein the first solute is a polyampholyte or a polyelectrolyte and the second solute is a polyhydroxy compound and/or an antichaotropic agent, wherein the polyampholyte comprises one or more covalently cross-linkable groups;
(b) allowing phase separation inside the droplet into a shell phase enriched in the first solute and a core phase enriched in the second solute, and gelation and/or precipitation in the shell phase to form an intermediate microcapsule;
(c) forming intermolecular covalent cross-links with the one or more covalently cross-linkable groups to form a microcapsule comprising a semi-permeable shell of covalently cross-linked polyampholyte or polyelectrolyte and a core.

In a fifth aspect, the present invention provides a method of reducing the viscosity of the core of the microcapsule of the first aspect, or the core of a microcapsule produced according to the fourth aspect, wherein the core comprises a polyhydroxy compound, the method comprising suspending the microcapsule in a liquid comprising a hydrolase enzyme, and allowing the hydrolase enzyme to diffuse into the core of the microcapsule and hydrolyze the polyhydroxy compound, thereby reducing the viscosity of the core.

In a sixth aspect, the present invention provides a method of lysing at least one cell in the core of the microcapsule of the first aspect or the microcapsule produced by the method according to the fourth or fifth aspects, the method comprising suspending the microcapsule in an external environment comprising a cell lysis reagent and/or a cell lysis enzyme, and allowing the cell lysis reagent or the cell lysis enzyme to diffuse into the core of the microcapsule and lyse the at least one cell.

In a seventh aspect, the present invention provides a method of removing one or more components from the core of the microcapsule according to the first aspect, or the core of the microcapsule produced by the method of any of the fourth to sixth aspects, the method comprising suspending the microcapsule in an external environment to create a concentration gradient between the core and the external environment, and allowing the one or more components to diffuse down the concentration gradient from the core to the external environment.

In an eighth aspect, the present invention provides a method of adding one or more component to the core of the microcapsule according to the first aspect, or the core of the microcapsule produced by the method of any of the fourth to seventh aspects, the method comprising suspending the microcapsule in an external environment to create a concentration gradient between the core and the external environment, and allowing the one or more components to diffuse down the concentration gradient from the external environment to the core.

In a ninth aspect, the present invention provides a microcapsule produced by the method of any one of the fourth to eighth.

In a tenth aspect, the present invention provides an in vitro method for culturing at least one cell encapsulated in and/or attached to the inner and/or outer surface of a microcapsule according to the first or ninth aspects, comprising incubating the microcapsule in an aqueous environment suitable to allow for cell survival, cell growth and/or cell proliferation.

In an eleventh aspect, the present invention provides an in vitro method for determining the phenotype (growth, viability, cellular response, metabolic activity, biological activity, protein binding activity, enzymatic activity) of encapsulated cell(s) in microcapsule, and/or attached to the inner and/or outer surface of a microcapsule, according to the first or ninth aspects, comprising incubating the microcapsule in an aqueous environment suitable for conducting a phenotypic assay readout.

In a twelfth aspect, the present invention provides an in vitro method for determining the genotype or genetic make-up (full or partial sequence of transcriptome or genome sequence, epigenetic profile, DNA methylation) of encapsulated cell(s) in microcapsule, and/or attached to the inner and/or outer surface of a microcapsule, according to the first or ninth aspects, comprising incubating the microcapsule in an aqueous environment suitable for conducting a genomics assay readout.

In a thirteenth aspect, the present invention provides a method of storing cells, comprising suspending the microcapsule according to the first or ninth aspects that comprises at least one cell in a cell storage medium comprising a cryoprotectant and freezing the cells.

In a fourteenth aspect, the present invention provides a method of releasing a core of a microcapsule, wherein the microcapsule is the microcapsule of the first or ninth aspects comprising at least one biological entity, the method comprising breaking the semi-permeable shell of the microcapsule.

In a fifteenth aspect, the present invention provides a method of delivering at least one biological entity to a subject for the treatment of a disease, a disorder or an injury in the subject, the method comprising administering a microcapsule according to the first or ninth aspects, to the subject, wherein the microcapsule comprises the at least one biological entity, optionally wherein the at least one biological entity is comprised in the core of the microcapsule.

In a sixteenth aspect, the present invention provides a microcapsule according to the first or ninth aspects for use in delivering a medical therapy, wherein the microcapsule comprises at least one biological entity for the medical therapy, optionally wherein the at least one biological entity is comprised in the core of the microcapsule.

In a seventeenth aspect, the present invention provides a microcapsule according to the first or ninth aspects for the manufacture of a medicament for delivering a treatment to a subject, wherein the microcapsule comprises at least one biological entity for the treatment.

In an eighteenth aspect, the present invention provides a kit for making the microcapsule according to the first aspect, the kit comprising:
(a) a polyhydroxy compound and/or an antichaotropic agent;
(b) a polyampholyte comprising one or more covalently cross-linkable groups; and optionally (c) a microfluidic chip.

Preferred features of the above aspects of the invention are defined in the dependent claims.

In one example the invention disclosed here provides a microcapsule composed of an elastic, covalently cross-linked shell, the liquid core, and containing an encapsulated cell, or more than one cell, whereas the said cell(s) may be maintained alive for extended periods of time, cultured, harvested, expanded and/or analyzed. The inner core of the said microcapsule can be in a liquid form, or in a hydrogel form and is preferably enriched in polyhydroxy compound belonging to the class of polysaccharides, oligosaccharides, carbohydrates, or sugars. The outer shell is composed of polyampholyte(s), and/or polyelectrolyte(s), (either natural or synthetic polymers). The outer shell may be composed of or enriched in the proteinaceous material composed of but not limited to the biopolymer belonging to the group of the extra-cellular matrix oligopeptides, peptides or proteins such as collagen, laminin, elastin, fibrin, proteoglycans, glycosaminoglycans, or their hydrolyzed forms (e.g. gelatin) thereof, or any combination thereof. As exemplified in one of the embodiments, the microcapsule shell is composed of polyampholyte (e.g., gelatin), whereas the core is composed of polyhydroxy compound (e.g., dextran). The microcapsule may comprise a cell or more than one cells, and/or biological (e.g., nucleic acids) and non-biological (e.g. colloid particles) species. The invention is best understood from the detailed descriptions when read in conjunction with the accompanying drawings, wherein FIG. 1 summarizes an example of the invention in the form of schematics. Note, various features of the drawing are not to-scale, which is a common practice. In one aspect, the microcapsule shell comprises those macromolecules (polyampholytes and/or polyelectrolytes) that undergo liquid-liquid and/or liquid-gel phase transitions. Upon phase transition, such as liquid-liquid phase separation or liquid-gel phase separation, the precursors (monomers, pre-polymer, polymer, polyampholyte chains, etc.) that will constitute microcapsule shell may self-assembly into a liquid shell and/or solidified shell, which may be further stabilized by cross-linking the monomers of the microcapsule shell. The microcapsule revealed in this invention may make use of the coacervation phenomenon, and/or liquid-liquid phase separation phenomena of aqueous two-phase systems.

According to the present invention microcapsule comprises a semi-permeable shell, which may be composed of a cross-linked polyampholyte gel. In one example, as exemplified in the disclosure the said microcapsule's shell is permeable to low molecular weight molecules, reagents, nutrients and compounds (smaller than 120±80 kDa) that may enter and leave the microcapsule, while simultaneously may prevent larger molecules from entering, or leaving, the same microcapsule. Indeed, the porosity and thus permeability of the shell and microcapsule may be altered by adjusting the concentration of the precursors (e.g., polyampholyte constituting the shell), adjusting the number of cross-linking sites in the shell, adjusting the number of cross-linking moieties (substitutions) on the precursors, altering polymerization conditions, and/or altering the composition of the shell with additives (e.g., adding PEG, polymers, proteins, polysaccharides, salts, etc.) and other situations. Due to semi-permeability of the microcapsule's shell the composition and solvent of the inner core of the said microcapsule may be altered, modified or changed by exposing the said microcapsule to a solution having a desirable biochemical composition, and allowing the molecules from the said solution to transverse the shell and by doing so alter, modify or change the inner content of the microcapsule. Importantly, the viscosity of the inner core may be modified and/or changed by hydrolyzing the polyhydroxy compound constituting the microcapsule's core without compromising the compartmentalization. In one non-limiting example the polyhydroxy compound may be hydrolyzed using hydrolase enzyme (e.g., dextranase, cellulase, agarose, etc.).

In one example, the invention describes a biocompatible microcapsule that carries an encapsulated cell, two cells, three cells or population of cells and may support the growth, expansion, harvesting and metabolic activity of the encapsulated cell(s). The encapsulated single-cell in a microcapsule may form, over time, the clonal population of cells and/or masses of cells, including but not limited to spheroids, organoids, tumoroids, tissues and other types of 3D cell assemblies (structures). Likewise, the capsules carrying two, three or more cells can be incubated in suitable cell culture conditions to enable formation of spheroids, organoids, tumoroids, tissues and other types of 3D cell assemblies.

The present invention also relates, in another example, to a microcapsule as a biocompatible compartment providing a support (substrate) for cells to attach to. For example, when the microcapsule's shell comprises a macromolecule that is oligopeptide, polypeptide, protein, modified protein or is of proteinaceous origin, then the microcapsule may serve as a substrate for cells to attach to. Likewise, when the microcapsule's shell comprises synthetic macromolecule (e.g., synthetic polymer) that includes amino acids and/or di-, tri-, oligo- or poly-peptide chains, the microcapsule may also serve as a substrate for cells to attach to. Indeed, cells may also attach to different types of macromolecules, if added to the shell, that are not proteinaceous origin such as polysaccharides, sugars, lipids, etc. When multiple cells are present within the microcapsule the cells may attach and arrange themselves at the inner surface of the said shell. In yet another example, the outer surface of the microcapsule's shell may serve as a substrate for cells present outside the microcapsule to attach to the microcapsule.

The present invention also relates in further examples to a microcapsule as a biocompatible compartment for cell co-culture. The plurality of cells encapsulated in plurality microcapsules may be in biochemical communication with each other while remaining enclosed within the microcapsules. The cells of one type encapsulated in first type microcapsules may be brought in biochemical communication with cells of other type also encapsulated in second type of microcapsules. Similarly, the microcapsules carrying cells of one type (e.g. tumor cell) may be suspended in a solution having a different type of cells (e.g., fibroblasts, immune cells) and allowing encapsulated cells and cells in suspension to biochemically communicate with each other. In yet another scenario, the cells in a suspension might attach to the outer surface of the microcapsule, and yet still remain physically separated from the encapsulated cells by the microcapsule's shell. Therefore, co-culture of two or more cell types becomes possible when using microcapsules of the invention revealed here. Upon biochemical communication between the cells it may be desirable to follow, monitor, record or analyze the phenotypic changes, and/or transcriptional and/or epigenetic and/or genetic changes of the cells. The biochemical communication between the cells may reveal new mechanisms by which tumors cells are recognized by the immune cells through the secreted biomolecules.

The present invention also relates in further examples to a microcapsule as a biocompatible compartment for cell co-culture within the microcapsule. For example, microcapsule having two cells of the same type (e.g., tumor cells) or different type (e.g. tumor cell and fibroblast, or tumor cell and immune cell, or dendritic cells and immune cell, etc.) could be cultured, harvested, monitored and/or analyzed. Likewise, a microcapsule carrying two or more cells of the same or different type could be cultured, harvested and, if desirable, allowed to form 3D cell assemblies (e.g., spheroids, organoids, tumoroids, etc.).

The present invention also relates in further examples to a microcapsule as a biocompatible compartment for the co-encapsulation of different cell types in the same microcapsule. For example, two different cell types can be co-encapsulated to allow two cell types to biochemically interact with each other, and/or establish cell-to-cell contact. So called two-cell screening assays may include but are not limited to cell cytotoxicity assay [34, 35], cancer cell and immune cell interaction assay [36], functional T-cell screening assay [37], natural killer assay [38], antibody secretion assay, and others cell-based assays.

The present invention also relates in further examples to a microcapsule as a biocompatible compartment for maintaining and/or analyzing cellular functions (e.g., growth, division, metabolic activity, gene expression, etc.) of the encapsulated cell(s) and physiological properties of 3D cell assemblies in the presence or absence a screening compound (e.g. chemical or biological compound). The phenotype and/or genotype of the encapsulated cell(s) may be evaluated using variety of molecular biology and biochemistry techniques known to a skilled person in the art. The microcapsules carrying encapsulated single-cell or population of cells may be subjected to multistep analytical procedures some non-limiting examples of which include pipetting, centrifugation, flow cytometry, molecular biology assays, biochemical reactions, genetic assays, analyzed and sorted using a fluorescence activated cell sorting instruments, etc., and yet still retain the encapsulated cell(s).

The entire microcapsules carrying cells may be loaded into water-in-oil droplets, nano-wells, micro-wells, wells of microtiter plates, along with reagents, DNA barcodes and/or enzymes necessary for phenotypic and/or genotypic analysis of the cells.

Upon a desirable step, the microcapsule may be decomposed and/or dissolved to release the encapsulated cells. In one non-limiting example the encapsulated cell(s) as well as 3D cell assemblies (e.g. spheroids) can be released by treating the capsules with proteolytic enzyme (e.g. protease).

As described in more detail below, the microcapsules of this invention, and the method of producing same, provide new features (e.g., high circularity and concentricity, cell adhesion to inner surface of the microcapsule), and solves some key challenges in the state-of-the-art such as capsule stability in different aqueous buffers, stability at different pH, stability in organic solvents, thermostability, biodegradability, resistance to bursting under internal pressure by growing cells, etc., thus making the microcapsules reported herein superior compartments for cell culture, harvesting, and analysis of both the cell(s) and nucleic acid derived therefrom. In particular, the microcapsules and methods of the present invention may provide the following advantages:

1) Composition. The microcapsules having the shell and core identified above can be reliably produced using the methods described herein, even at larger diameters of greater than 60 μm. In particular embodiments, where the polyampholyte is a polymer comprising peptide bonds, the shell of the microcapsule can be readily digested e.g. so as to release any biological entity comprised in the microcapsule, with a protease enzyme. In this manner harsh chemical conditions (e.g. alkaline treatment) which may damage the biological entity can be avoided. This can be particularly important in embodiments where the biological entity is a cell and cell viability after release from the microcapsule is a relevant consideration. In contrast to some more complex microcapsules of the prior art, the invention reported here reveals microcapsule comprising the shell (envelope) that is composed of a single layer (envelope). Further, in some embodiments where the core is composed of a polyhydroxy compound (e.g. dextran), this can be hydrolyzed upon enzymatic treatment (e.g. dextranase) and such treatment will not dissolve the microcapsule shell, allowing the internal viscosity of the microcapsule to be reduced while still maintaining the integrity of the microcapsule. This can be particularly advantageous where reducing the viscosity of the core of the microcapsule can improve the efficiency of the methods being performed in the microcapsule, e.g. nucleic acid analysis, cell growth. It can also be advantageous after the biological entity has been released from the microcapsule in order to assist in separating the biological entity from the polyhydroxy compound of the core before further downstream method steps are performed.

2) Chemical Stability. Some previously reported capsules comprise a gelled shell that is cross-linked non-covalently, e.g. alginate chains cross-linked via metal complexation (e.g. with calcium). Such gels, while stable in certain aqueous buffers, provide limited long-term stability in cell culture conditions and physiological conditions [39]. Such gels are prone to dissolution (and thus loss of encapsulated cells) due to release of divalent ions into the surrounding media due to exchange reactions with monovalent cations. Such gels also dissolve in the presence of phosphate ions, citrate and other chemical compounds, ions or salts, that can displace, or chelate the metal cations participating in the complexation. The capsules reported by others are also sensitive to pH. For example, it is known that calcium-alginate hydrogel may decompose upon lowering pH below 7.0 [40], which may occur during cell culture and harvesting due to metabolic activity of encapsulated cells. As such the capsule reported previously do not provide sufficiently broad conditions for culturing and analyzing encapsulated cells, and restricts capsule use only in certain aqueous buffers and cell growth medium (e.g. deprived of phosphate ions), and prevents the use of some reagents to perform biochemical or biological reaction (e.g., EDTA, acetate, cations). In contrast, because the shell of microcapsules reported here is covalently cross-linked it cannot be dissolved in the presence of different salts, cations, chelating agents or change in pH, and overall provides superior stability in a higher variety of aqueous buffers than what is possible to achieve using state-of-the-art capsules.

3) Thermal Stability. The microcapsules disclosed in this invention are thermostable and do not degrade upon heating at elevated temperatures (e.g., 95° C.), allowing the microcapsules to be utilized to perform methods such as PCR or incubation at raised temperatures to denature proteins.

4) Physical Stability. The microcapsules disclosed in this invention withstand multi-step procedures such as pipetting, flow cytometry, FACS or centrifugation at 10.000 g and higher (where g is known as g-Force or Relative Centrifugal Force). Because of the high stability the microcapsules of this disclosure can withstand higher osmotic pressure differences than the capsules reported in the state-of-the-art. As result of this improved stability the disclosed microcapsules do not burst when 3D cell culture reaches confluency inside the microcapsule and exerts pressure on the microcapsule shell. The volume of microcapsules reported in this disclosure can increase 8-times and still retain the encapsulated cells, or 3D cell assemblies. For example, it has been recognized (see FIG. 14, US 2015/0017676A1) that once encapsulated cells reach confluency and exerts pressure on the shell, the microcapsule will eventually burst. Some capsules previously reported have a ratio h/R at about 0.1 (where h is shell thickness, and R microcapsule's radius) and could withstand radial deformation up to 30%. However, the capsules reported in the present disclosure can withstand more than 30% degree of radial deformation when h/R ratio value is at about 0.1, or even below 0.1.

5) Concentricity and Circularity. In comparison to the existing state-of-the-art capsules, the concentricity and circularity of the disclosed microcapsules is of higher quality, which is important feature for ensuring even diffusion of nutrients and reagents into the microcapsule core, as well as for uniform conditions for cell culture. High concentricity and circularity may also be important for reducing technical biases and measurement artifacts that may arise when working with microcapsules having uneven shell, or ellipsoidal shape. High concentricity and circularity may also be important for performing high-throughput assays, digital quantification, gene expression analysis, phenotypic analysis, genotypic analysis and for measuring cell response to (bio)chemical compounds. Some previously reported microcapsules have very broad circularity, C, varying between 0.4-1.0, with majority of microcapsules (55%) having circularity between 0.4 and 0.8, and remaining fraction (45%) having circularity between 0.8-1.0. In the present disclosure over 95% of microcapsules have extremely high circularity with a very narrow distribution, C=0.9±0.1. Where the circularity, C, is defined as a ratio of the minor axis (R min) over the major axis (R max) of the ellipse adjusted to the external edge of the projected equatorial section. The radius, R, of the microcapsule is defined as R=√(square root over (S/π)), wherein S is the equatorial transverse surface of the capsule. Moreover, the concentricity, O, of the microcapsule defined as O=(W_min/W_max)*100%, wherein W_min is thinnest part of the shell and W_max is the thickest part of the shell, in the present disclosure shows high values (O≥66%). The concentricity of the state-of-the-art capsules is typically below 66%.

6) Cell adhesion. The microcapsule's shell provides a substrate (support) for both encapsulated cells and cells in suspension (that reside outside the microcapsule) to adhere to the shell

7) Permeability. In one example, the invention disclosed here discloses a microcapsule comprising covalently cross-linked shell that is permeable to double stranded DNA fragments shorter than approximately 200 bp. and not permeable to double stranded DNA fragments of approximately 200 bp or longer. In further examples, the present invention discloses a microcapsule comprising a covalently cross-linked shell that is permeable to proteins of approximately 100 kDa. In particular, this allows reverse transcriptase reactions and PCR reactions to be performed on nucleic acids retained in the microcapsule while the enzymes for these reactions can diffuse into and out of the microcapsule.

8) Cell cytotoxicity. Previously disclosed capsules are produced using an ionic surfactant, which interferes with cell viability. The method of the invention disclosed here can be performed using non-ionic surfactants, thus solving the cytotoxicity problem, and accordingly does not interfere with cell viability. Even at relatively high concentration of the surfactant (>1% w/v) over 90% of cells remain alive after encapsulation.

### BRIEF DESCRIPTION OF THE DRAWINGS

To assist understanding of the present disclosure and to show how embodiments may be put into effect, reference is made by way of example to the accompanying drawings described below. It should be noted that in the schematics provided various aspects are not drawn to scale.

FIG. 1. Schematics showing examples of the microcapsules of the invention. Examples of microcapsules of the disclosure comprising a cross-linked shell enriched/composed of polyampholyte, a core enriched/composed of polyhydroxy compound or salts, and a cell. (A) A microcapsule comprising a shell enriched/composed of polyampholyte (1), liquid core enriched/composed of polyhydroxy compound or salt-rich solution (2) and encapsulated cell (3). (B) A microcapsule comprising a shell enriched/composed of polyampholyte (1), semi-liquid or hydrogel-based core composed of polyhydroxy compound (4) and encapsulated cell (3).

FIG. 2. Schematic showing an example of the encapsulated cell culture into 3D cell assemblies. A microcapsule carrying a single-cell can be incubated in suitable conditions to allow the encapsulated cell to divide and form a 3D cell assembly. 1—shell, 2—core, 3—cell, 4—incubation, 5—complex 3D cell structure (e.g. spheroid, organoid, assembly, or tissue).

FIG. 3. Schematics showing examples of the encapsulated cell culture into 3D cell assemblies. A microcapsule carrying more than once of different type can be incubated in suitable conditions to allow the encapsulated cell to divide and form 3D cell assemblies composed of poorly organized cells (A) or organized layers of cells (B). 1—shell, 2—core, 3—cell of type A, 4—cell of type B, 5—incubation.

FIG. 4. Schematics showing examples of the cell culture into various 3D cell assemblies. A microcapsule carrying one or more than one cell may be incubated in suitable conditions to allow the cell(s) to divide and form 3D cell assemblies composed of (A) cells attached to the inner surface of the shell; (B) cells attached to the outer layer of the shell; (C) cells attached to the inner and outer surface of the same microcapsule; (D) cells attached to the inner surface of the shell and forming multiple layers (sheets); (E) cells attached to the inner surface of the shell and forming multiple layers (sheets); (F) cells attached to the inner and outer surface of the same microcapsule, and forming multiple layers. 1—shell, 2—core, 3—encapsulated cell, 4—cell outside the microcapsule.

FIG. 5. Schematics showing examples of the cell-based assays exploring the interactions between the cells. A microcapsule can be used as a microcompartment for studying biological and biochemical interactions between the cells, within and outside the microcapsule or between the microcapsules. The communication and/or interactions between the cells may affect their biological response and/or phenotype. Two cells can be isolated into the same microcapsule and allowed to communicate with each other bidirectionally (A) or unidirectionally (B). Cells isolated in microcapsules can communicate with other cells present outside the microcapsule bidirectionally (C) or unidirectionally (D). Cells isolated in microcapsules can communicate with each other bidirectionally (E) or unidirectionally (F). 1 microcapsule, 2—cell of type A, 3—cell of type B, 4—molecules produced by cell of type A, 5—molecules produced by cell of type B. Arrow indicates the direction of the communication between the cells.

FIG. 6. Schematics showing examples of the cell-based assays exploring the interactions between the cells. A microcapsule can be used as a microcompartment for studying biological and biochemical interactions between the cells within and outside the microcapsule or between the microcapsules. The communication and/or interactions between the cells may affect their biological response and/or phenotype. (A) A 3D cell culture (e.g., spheroid) present in a microcapsule can be allowed to communicate with individual cells isolated in another microcapsule; (B) A 3D cell culture (e.g., spheroid) in one microcapsule can be allowed to communicate with a 3D cell culture (e.g., spheroid) in another microcapsule. (B) A 3D cell culture (e.g., spheroid) in a microcapsule can be allowed to communicate with cells forming a monolayer in another microcapsule; (D) Cells forming a monolayer in first microcapsule can be allowed to communicate with cells present in second microcapsule; (E) Cells forming a monolayer in first microcapsule can be allowed to communicate with cells forming a monolayer in a second microcapsule. 1—microcapsule, 2—cell of type A, 3—cell of type B, 4—molecules produced by cell of type A, 5—molecules produced by cell of type B. Arrow indicates the direction of the communication between the cells.

FIG. 7. Schematics showing examples of the cell-based assays exploring the interactions between the cells. A microcapsule can be used as a microcompartment for studying biological and biochemical interactions between the cells within and outside the microcapsule or between the microcapsules. The communication and/or interactions between the cells may affect their biological response and/or phenotype. (A) A cell attached to the outer surface of the shell of the microcapsule comprising a cell can communicate with each other; (B) A cell attached to the inner surface of the shell of the microcapsule can communicate with a cell outside the microcapsule. (C) Multiple cells attached to the outer surface of the shell of the microcapsule comprising a cell can communicate with each other; (D) Multiple cells attached to the outer and inner surface of the same microcapsule and forming monolayers can communicate with each other; (E) Multiple cells attached to the outer surface of the shell and forming a monolayer may communicate with multiple cells present inside the same microcapsule. (F) Multiple cells attached to the outer and inner surface of the same microcapsule and forming multilayers can communicate with each other. 1—microcapsule, 2—cell inside the microcapsule, 3—cell outside the microcapsule, 4—molecules produced by cell(s) inside the microcapsule, 5—molecules produced by cell(s) outside the microcapsule. Arrow indicates the direction of the communication between the cells.

FIG. 8. Schematics showing examples of the operation of microfluidics system for generation of microcapsules. 1—an inlet for aqueous phase enriched in shell-forming compound; 2—an inlet for aqueous phase enriched in core-forming compound; 3—carrier oil, 4—emulsion collection outlet. (FIG. 8A) Schematics of microfluidics chip and its operation. (FIG. 8B) Example of the microfluidics chip for the generation of capsules. (FIG. 8C) Digital micrographs. Scale bars, 100 μm

FIG. 9. Photograph of mammalian cells encapsulated in water-in-oil droplets. The cells encapsulated in water-in-oil droplets composed of the 12% (w/v) PEGDA (MW 2000) and 15% (w/v) Dextran (MW 500K) polymers distributed at the PEGDA/Dextran interface and/or PEGDA phase. Arrows indicate the water-in-oil droplets having a cell. The diameter of the capsule is approximately 75 μm.

FIG. 10. Photographs of mammalian cells encapsulated in PEGDA/dextran capsules having a thin shell. The cells (black arrows) escaped compartmentalization during capsule generation. The shell is between 4 and 10 μm thick.

FIG. 11. Photographs of mammalian cells encapsulated in PEGDA/dextran capsules having a thick shell. The PEGDA/Dextran capsules with a thick (˜20 μm) shell lost concentricity and the compartmentalized cells (black arrows) tend to escape the compartmentalization through the thinner part of the shell.

FIG. 12. Schematics showing an example of microcapsule generation and analysis cell encapsulation. (A) First a plurality of cells is encapsulated in plurality of water-in-oil droplets comprising shell forming solution and core forming solution. In a typical scenario, the water-in-oil droplets having liquid core and liquid shell are converted to intermediate-microcapsules having a liquid core and a solidified shell. The intermediate-microcapsules are converted to the final microcapsule by cross-linking the solidified shell. In some scenarios, the water-in-oil droplets having liquid core and solidified (gelled) shell are produced during droplet generation without obvious liquid shell formation. As described in the main text different approaches can be employed to form polymerized (cross-linked) shell. The capsules having polymerized shell can be subjected to chemical, physical or enzymatic treatments (e.g. capsules can be dispersed in cell culture to enable encapsulated cell division and growth). 1—suspension of cells, 2—encapsulated cell, 3—cell encapsulation, 4—an aqueous phase enriched in shell forming compound (e.g., gelatin methacrylate); 5—an aqueous phase enrich in core-forming compound (e.g., dextran); 6—carrier oil, 7—water-in-oil droplet collection, 8—liquid core, 9—gelled (solidified) shell, 10—polymerization (cross-linking) of the shell, 11—aqueous buffer, 12—covalently cross-linked shell. (B) The still microscopy images showing cell encapsulation and water-in-oil droplet collection. Scale bar, 100 μm. (C) Digital images and schematics of two-step process of microcapsule generation, which involves water-in-oil droplet cooling at 4° C. to induce the liquid shell solidification into a gelled shell followed by the solidified shell cross-linking by chemical, physical or biological means. The final microcapsule comprises semi-permeable membrane (shell) enriched in polyampholyte and liquid or semi-liquid core enriched in polyhydroxy compound.

FIG. 13. Photographs showing microcapsule generation using gelatin with a different degree of methacrylate substitution. Capsules were generated using gelatin/dextran blend where gelatin contained different percentage of methacrylate substitution. For each test 3% (w/v) of gelatin polymer with of a given degree of substitution, and 15% (w/v) dextran (MW ˜500k) were used. (A) gelatin with 0% degree of substitution, (B) GMA with 40% degree of substitution, (C) GMA with 60% degree of substitution, (D) GMA with 80% degree of substitution. Scale bars, 100 μm.

FIG. 14. Photograph showing microcapsule generation using GMA with a low-degree of substitution. Capsules were generated using 5% (w/v) GMA with 40% degree of substitution and 15% (w/v) dextran (MW ˜500k). Scale bar, 100 μm.

FIG. 15. Schematic showing examples of microcapsule generation using different polymerization approaches. (A) Capsule generation process where cross-linking of the capsule shell was performed during droplet generation step by exposing liquid droplets to photo-illumination. (B) Capsule generation process where cross-linking of the capsule shell was performed by exposing off-chip collected emulsion to photo-illumination. (C) Capsule generation process where at first the capsules' shell was solidified during temperature-induced gelation process, and only then cross-linked by photo-illumination. (D) Capsules, where the shell was polymerized following emulsion collection off-chip, incubation at 4° C. to induce gelation of the shell, dispersed solidified capsules in aqueous buffer only then cross-linked via light-induced polymerization. 1—photo-illumination, 2—capsule collection off-chip, 3—carrier oil, 4—liquid core, 5—chemically cross-linked shell, 6—capsule dispersion in aqueous buffer, 7—water phase, 8—generation of water-in-oil droplets, 9—liquid shell, 10—droplet incubation at low temperature (e.g. 4° C.) to induce the solidification of the shell, 11—solidified shell.

FIG. 16. Microcapsule generation using temperature-induced and/or light-induced polymerization. Photographs show capsules dispersed in aqueous buffer after polymerization of capsules' shell by temperature-induced gelation and/or light-induced cross-linking. (A) Capsules were generated by cross-linking capsule shell during droplet generation step by exposing droplets to photo-illumination and then dispersed in an aqueous buffer. (B) Capsules, where the shell was polymerized by photo-illumination immediately after emulsion collection off-chip and then dispersed in an aqueous buffer. (C) Capsules, where the shell was polymerized following emulsion collected off-chip, incubation at 4° C. to induce gelation (solidification) of the shell and cross-linking via light-induced polymerization, and then dispersed in an aqueous buffer. (D) Capsules, where the shell was polymerized following emulsion collection off-chip, incubation at 4° C. to induce gelation of the shell, dispersing capsules in an aqueous phase and only then cross-linking via light-induced polymerization. Scale bars, 100 μm.

FIG. 17. Microcapsule generation using chemical agent-induced polymerization. Photographs show capsules dispersed in an aqueous buffer after polymerization of capsules' shell by chemical agent induced cross-linking and the combination of temperature-induced gelation and chemical agent induced cross-linking. (A) Capsules were generated using GMA/dextran blend where GMA phase was supplemented with 0.3% (w/v) APS, and carrier oil phase was supplemented with 0.4% (v/v) TEMED. The emulsion was collected off-chip and incubated at room temperature for 2 hours to allow sufficient period of time for shell polymerization to occur. Shown are the resulting capsules suspended in an aqueous buffer. (B) The water-in-oil droplets containing GMA/dextran blend were generated and collected off-chip, and incubated at 4° C. for 30 min to induce solidification of the shell. The solidified capsules were then resuspended in an aqueous buffer containing polymerization initiators (0.3% (w/v) APS and 0.4% (v/v) TEMED) and incubated at room temperature for 2h to induce chemical cross-linking of capsules' shell. Shown are the resulting capsules suspended in an aqueous buffer. Scale bars, 100 μm.

FIG. 18. Photographs showing microcapsule generation using natural (a non-modified) polyampholyte. The microcapsules comprising a polyampholyte comprising amine and amide residues were cross-linked using genipin and microbial transglutaminase (mTG). The microcapsules comprising a natural polyampholyte (gelatin from porcine skin) were generated as described in the Example 4. (A) Performing a cross-linking reaction with 0.5% genipin for 1h at 4° C. is not sufficient to obtain stable and intact microcapsules. (B) Performing a cross-linking reaction with 0.5% genipin for 24 h at 4° C. produced thermo-resistant microcapsules that remained intact after heating at 50° C. for 10 min. (C) Performing a cross-linking reaction with 0.3 U/mL of mTG for 30 min at 23° C. was sufficient to obtain stable microcapsules that remained intact after heating at 50° C. for 10 min. (D) Performing a cross-linking reaction with 0.3 U/mL of mTG for 60 min at 4° C. was sufficient to obtain stable microcapsules that remained intact after heating at 50° C. for 10 min. The average capsule size ˜50 μm. The grid size 200×200 μm\hat{ }2.

FIG. 19. Photographs showing microcapsule production using variety of polyhydroxy compounds. Capsules were generated using a mixture of GMA and polyhydroxy compounds. (A) Capsules composed of GMA and hydroxyethyl-cellulose, solidified and polymerized at ˜4° C. temperature. (B) Capsules composed of GMA and Ficoll PM400, solidified and polymerized at ˜4° C. temperature. Scale bars, 100 μm.

FIG. 20. Photograph of microcapsules having a core comprising salts. Capsules were generated using a mixture of GMA and ammonium sulphate. Scale bar, 100 μm.

FIG. 21. Photographs showing of microcapsules having different diameter. (A) Capsules having a diameter of 35 μm, (B) Capsules having a diameter of 60 μm, (C) Capsules having a diameter of 180 μm, (D) Capsules having a diameter of 24 μm. Scales bars, 100 μm.

FIG. 22. Photographs showing microcapsule size control by temperature. (A) Capsules composed of GMA and dextran, were solidified and photo-polymerized at −4° C. temperature. (B) Capsules composed of GMA and dextran, were solidified at −4° C. temperature and photo-polymerized after 15 minutes incubation at room (˜22° C.) temperature. (C) Capsules composed of GMA and hydroxyethyl-cellulose, were solidified and photo-polymerized at ˜4° C. temperature. (D) Capsules composed of GMA and hydroxyethyl-cellulose, were solidified at ˜4° C. temperature and photo-polymerized after 15 minutes incubation at room (˜22° C.) temperature. (E) Capsules composed of GMA and Ficoll PM400, were solidified and photo-polymerized at ˜4° C. temperature. (F) Capsules composed of GMA and Ficoll PM400, were solidified at ˜4° C. temperature and photo-polymerized after 15 minutes incubation at room (˜22° C.) temperature. Scale bars, 100 μm.

FIG. 23. Increasing the concentration of the shell-forming precursor leads to capsules with thicker shells. The capsules were generated by emulsifying 5% (w/v) GMA with 15% (w/v) dextran solutions followed by physical gelation and light-induced cross-linking of the shell either at ˜4° C. (panel A) or ˜22° C. temperature (panel B). (A) The photograph shows ˜68 μm diameter capsules having 6.5 μm shell and 55 μm core. (B) The photograph shows ˜82 μm diameter capsules having 6 μm shell and 70 μm core. Scale bars, 100 μm.

FIG. 24. Single-cell isolation in capsules. Cell retention comparison in droplets and in capsules. Boxplots (FIG. 24A) and bar plots (FIG. 24B) representing mammalian cell retention in droplets and capsules. Independent samples t-test showed there is no statistically significant difference of cell occupancy in droplets and capsules. (p=0.2281). Cell occupancy in droplets is 8.2±1.6, cell occupancy in capsules is 8.8±1.3. In both FIG. 24A and FIG. 24B values for retention in droplets is shown on left with the values for retention in capsules shown on the right.

FIG. 25. Photographs showing results of comparison of long-term cell culture in microcapsules and in beads. The formation of 3D cell assemblies (e.g., spheroids) originating from a single mammalian cell was conducted using microcapsules (based on gelatin/dextran composition), gelatin beads and agarose beads. The K-562 cells were loaded to microcapsules or beads as described in Example 11. (A) Cells cultured in microcapsules comprising gelatin/dextran composition were retained in microcapsules during 7 days of cell culture without bursting the microcapsules. (B) Cells cultured in gelatin-based beads escaped compartmentalization after 4-7 days of cell culture. (C) Cells cultured in agarose-based beads escaped the compartmentalization after 3 days of culture.

FIG. 26. Photographs showing results of a 3D cell culture in microcapsules. The 3D cell culture over time of human breast adenocarcinoma cells (MDA-MB-231) in microcapsules. Encapsulated cells attached to the inner surface of the microcapsules during the 12 hours of cell culture and subsequently grew into more complex 3D cell assemblies over long-term culture lasting 11 days. Scale bar, 50 μm.

FIG. 27. Photographs showing results of a 3D cell culture in microcapsules. The 3D cell culture over time of human alveolar basal epithelial cells (A549) in microcapsules. Encapsulated cells attached to the inner surface of the microcapsules during the 12 hours of cell culture and subsequently divided and formed more complex 3D cell assemblies over long-term culture lasting 11 days. Scale bars, 50 μm.

FIG. 28. Photographs showing results of cell culture on microcapsules. The cells in suspension may attach to the microcapsules when cells and microcapsules are mixed together. Human breast adenocarcinoma cells (MDA-MB-231) were incubated with microcapsules in 1×DMEM medium supplemented with 10% FBS and 1× Penicillin-Streptomycin for 12 hours. The cells started to adhere to the outer surface of the microcapsules during 2 hours of incubation and in some cases almost completely covered the surface of the capsules after 12 hours of incubation. Scale bars, 50 μm.

FIG. 29. Photographs and schematics of selected examples of cell-based co-culture assays using microcapsules. (A) A co-culture assay may involve the cells of interest inside microcapsules and cells present in the same suspension (outside the microcapsule). The cells present inside and outside of the microcapsule may communicate biochemically via soluble factors. (B) A co-culture assay may involve the cells of interest present inside microcapsules and cells attached to the outer surface of the same microcapsule. The cells present inside and outside of the microcapsule may communicate biochemically via soluble factors. (C) A co-culture assay where the two cells are present in the same microcapsule. (D) A co-culture assay where the cells present in different microcapsules are co-cultured in the same mix. (E) A co-culture assay where the cells attach to the outer surface of the microcapsule, and where the same microcapsule carries a cell (or several cells). The cells present inside and outside of the microcapsule may communicate biochemically via soluble factors. (F) A cell assay where the cell attaches to the outer surface of one or more than one the microcapsule and may bring two or more microcapsules in close proximity. 1—cell inside a microcapsule, 1A—cell of type A, 1B—cells of type B, 2—cell outside a microcapsule, 3—microcapsule, 4—molecules produced by cell of type A, 5—molecules produced by cell of type B. Arrow indicates the direction of the communication between the cells.

FIG. 30. Photographs and schematics of selected examples of cell-based co-culture assays using microcapsules. (A) The cell co-culture assay may involve the cells attached to the outer surface of the microcapsule and forming a layer (e.g., monolayer, multilayer), whereas the cells inside the microcapsule may form a 3D cell assembly (e.g., spheroid, tissue, etc.); (B) the formation of a 3D cell assembly comprising multiple layers of cells; (C) the formation of a 3D cell assembly comprising a layer of cells attached to the inner surface of the microcapsule; (D) the cell co-culture assay where a 3D cell assembly (e.g., spheroid) in one microcapsule is incubated in the suspension having microcapsules comprising a single-cell (or several cells), and whereas the cells inside the microcapsules may communicate biochemically via secreted factors; (E) the cell co-culture assay where the 3D cell assembly (e.g., spheroid) of one cell type present in one microcapsule is suspended in the suspension having microcapsules comprising the 3D cell assembly of another cell type, and whereas the cells inside the microcapsules may communicate biochemically via secreted factors; (F) the cell co-culture assay where the 3D cell assembly comprising cells attached to the outer surface of the microcapsule and cells residing inside the same microcapsule are assayed in the mix comprising the other type of cells which also form a 3D cell assembly comprising cells attached to the outer surface of the microcapsule and cells residing inside the same microcapsule, and whereas the cells inside the microcapsules may communicate biochemically via secreted factors. 1—cell inside a microcapsule, 2—cell outside a microcapsule, 3—microcapsule, 4— molecules produced by present outside the microcapsule, 5—molecules produced by cell present inside the microcapsule. Arrow indicates the direction of the communication between the cells.

FIG. 31. Photographs showing cell release from microcapsules. The microcapsules comprising a shell composed of cross-linked GMA, and having HEK293 cells inside, were treated with collagenase A to release the encapsulated cells over the course of 110 seconds. Scale bars, 100 μm.

FIG. 32. Photographs showing cell release from microcapsules. The microcapsules comprising a shell composed of cross-linked GMA, and having K-562 cells inside, were treated with collagenase A to release the encapsulated cells over the course of 110 seconds. Scale bars, 100 μm.

FIG. 33. Photographs showing cell viability during cell culture and harvesting. The microcapsules comprising K-562 were incubated in a cell growth medium for extended period of time, and at selected time points the viability of cells was evaluated using fluorescent dyes (SYTO 9 and Ethidium homodimer-1). Cells remain highly viable for a few days of culture and few dead cells appear on Day 8 due to cell confluency, lack of nutrients and/or other factors. Fluorescence visible in second column (“SYTO 9”) indicates live cells, and fluorescence vising in third column (“EthD-1”) indicates dead cells. Scale bars, 100 μm.

FIG. 34. Photographs showing fluorescence analysis of 3D assemblies in microcapsules. The PFA-fixed cells were stained for actin and nuclei, using phalloidin and DAPI dyes, respectively revealing the cellular structure of complex 3D cellular structure. Scale bars, 50 μm.

FIG. 35. Photograph of a 3D cell culture in microcapsules treated with dextranase. The 3D cell culture of Hela cells at Day 5 in microcapsules treated with dextranase enzyme. Scale bar 70 μm.

FIG. 36. Microcapsules comprising a composite mixture of polyampholytes. Bright field microscopy images of microcapsules comprising a shell composed of 2% gelatin methacrylate and 2% gelatin from porcine skin. The ratio h/R is about 0.18 (where h is shell thickness, and R microcapsule's radius) and the average concentricity is approximately 75%. Scale bar 40 μm.

FIG. 37. Photographs and box plots showing microcapsules are compatible with bacteria culture. Escherichia coli MG1655 cells were harvested inside ˜40 μm microcapsules suspended in LB-Miller containing 0.1% (w/v) Pluronic F-68 for extended periods of time. Bacteria cells formed isogenic microcolonies derived from single-cells. Microcapsules comprised a shell composed of 2% gelatin methacrylate and 2% gelatin from porcine skin. Scale bars, 50 μm.

FIG. 38. Photographs and box plots showing microcapsules are compatible with unicellular organism culture. Saccharomyces cerevisiae were harvested in 55 μm diameter microcapsules over extended periods of time by suspending microcapsules in YPD containing 0.1% (w/v) Pluronic F-68. Yeast cells divided very efficiently inside the microcapsules and formed clonal micro-colonies derived from single-cells. Microcapsules comprised a shell composed of 2% gelatin methacrylate and 2% gelatin from porcine skin. Scale bars, 50 μm.

FIG. 39. Photographs showing adherent cell culture in microcapsules comprising a composite mixture of polyampholytes. The human colon derived cells (SW620) were harvested in microcapsules those shell comprises 2% gelatin methacrylate and 2% gelatin from porcine skin. SW620 cells divided and expanded inside the microcapsules and formed 3D cell structures after 4 days. Scale bars, 50 μm.

FIG. 40. Photographs of suspension cell culture in microcapsules comprising a composite mixture of polyampholytes. The bone marrow cells (K-562) were harvested in microcapsules those shell comprises 2% gelatin methacrylate and 2% gelatin from porcine skin. K-562 cells divided and expanded inside the microcapsules and formed spheroids after 4 days of cell culture. Scale bars, 50 μm.

FIG. 41. Photographs of microcapsules stability evaluation at different chemical and physical conditions. The microcapsule stability was evaluated by incubating microcapsules at different buffer for 60 min unless stated otherwise. The said conditions include, capsules stability evaluation in MQ-water, 1× Dulbecco's phosphate-buffered saline (DPBS) buffer, 1×DPBS buffer containing 1% Pluronic F68, 1× Hanks' Balanced Salt Solution (HBSS) buffer, 1× saline-sodium citrate (SSC) buffer, 10 mM Tris-HCl, 100 mM NaCl, 5% DMSO in water, 25% glycerol, 70% ethanol, 90% methanol, 90% acetone, 2M Acetic acid for 30 min, 2M NaOH for 15 min. Scale bars, 100 μm.

FIG. 42. Photographs of microcapsules stability evaluation after freezing at −20° C. or −80° C. The microcapsules were added to a given solution and then transferred to either −20° C. or −80° C. and incubated for 14 hours or longer. Following incubation, the microcapsules were centrifuged, supernatant discarded and microcapsules resuspended in 1×PBS supplemented with 0.1% Pluronic F68 and evaluated under bright field microscopy. The solutions in which microcapsules were suspended and cooled down at −20° C. or −80° C. included: water, 1×DPBS buffer containing 0.1% Pluronic F68, 5% DMSO, 25% glycerol, 70% ethanol, 90% methanol, 90% acetone. In all conditions tested microcapsules retained core/shell structure. Scale bars, 100 μm.

FIG. 43. Photographs of microcapsules stability after centrifugation and sonication. (A) microcapsules after centrifugation at 3000 g for 15 min; (B) microcapsules after centrifugation at 20000 g for 15 min; (C) microcapsules after sonication (1 second=65 joules) using VTUSC3 (Velleman) instrument for 15 minutes (D) microcapsules after sonication after 15 cycles (30 seconds ON and 30 seconds OFF). Microcapsules were 70 μm in diameter.

FIG. 44. Graphs and photograph of microcapsule analysis using FACS instrument. The FITC-dextran labelled capsules were analyzed on FACS instrument using forward scatter (FSC), side scatter (SSC) and fluorescence. (A) Side vs. forward scatter plot of capsule sample. (B) The capsules gated on the forward vs. side scatter plot were re-plotted on the FSC vs. fluorescence scatter plot. (C) The capsules gated on the forward vs. side scatter plot were re-plotted on the SSC vs. fluorescence scatter plot. (D) The digital photograph of FITC-dextran labelled capsules used in the flow cytometry analysis. Scale bar, 100 μm.

FIG. 45. Agarose gel photograph showing retention of DNA fragments inside the semi-permeable compartments. The microcapsules subjected to different conditions (see Example 20 for more details). GeneRuler 100 bp Plus DNA ladder (cat no. SM0321, ThermoFisherScientific) was encapsulated in water-in-oil droplets and processed as follow. The encapsulated DNA ladder was released immediately after droplet collection off-chip showing that there is no preferential DNA fragment loss during encapsulation process (well #1). The intermediate-microcapsules also retained encapsulated DNA fragments and shown now preferential loss (well #2). The microcapsules incubated at room temperature (well #3 and #5) or incubated at 50° C. for 30 min (well #4 and #6) show the same degree of DNA fragment retention. The capsules treated with dextranase (well #5 and #6) did not affect DNA fragment retention. Microcapsules were broken by treating with protease, the released material was combined with DNA loading dye and individual samples were loaded in agarose wells for DNA electrophoresis. Well #M indicates a well having GeneRuler 100 bp Plus DNA ladder.

FIG. 46. Photographs of epifluorescence microscopy analysis of capsules after multiplex RT-PCR. The first (top) row shows multiplex RT-PCR results on capsules carrying a mixture of K562 and HEK293 cells. The second row shows multiplex RT-PCR results on capsules carrying K562 cells. The first column shows bright field images. The second column shows Alexa Fluor 647 dye fluorescence images corresponding to ACTB positive capsules. The third column shows Alexa Fluor 488 dye fluorescence images corresponding to PTPRC positive capsules. The fourth column shows Alexa Fluor 555 dye fluorescence images corresponding to YAP positive capsules. Fifth column shows merged (superimposed) images demonstrating cell specific markers (PTPRC and YAP) overlapping with ACTB expression. Scale bars, 50 μm.

FIG. 47. Photographs of microcapsules after whole genome amplification of single-cells. The single-cells (K-562 cell line) were isolated in microcapsules, lysed and the genome was amplified using phi29 DNA polymerase. After whole genome amplification (WGA) the microcapsules were stained with fluorescent dyes to evaluate the reaction yields. A) Photographs of single-cell whole genome amplification product stained with SYBR Green I dye. B) Photographs of single-cell whole genome amplification product stained with SYTO-9 dye. After WGA reaction the majority of microcapsules expanded in size due to increased levels of DNA present in the microcapsules. Scale bars, 100 μm.

FIG. 48. Photographs of cell culture in microcapsules that were previously cryopreserved. The A-549 cells isolated in microcapsules were cryopreserved in liquid nitrogen, stored for 1 week, recovered and cultivated in cell culture for 14 days. A) Time-lapse images of A549 cells before cryopreservation (16-18h) and after freezing/thawing at day 2, 6 and 14. Scale bars, 50 μm. B) Cell viability evaluation after 14 days in culture post cryopreservation. Scale bars, 100 μm.

FIG. 49. Photographs of cell spheroid culture in microcapsules that were previously cryopreserved. The A-549 cells isolated in microcapsules were cryopreserved in liquid nitrogen, stored for 1 week, recovered and cultivated in cell culture for 14 days. A) Microscopy analysis showing microcapsule and 3D cell structure integrity before cryopreservation and after freezing/thawing. The cryopreserved spheroids were stored in a liquid nitrogen for 1 week before thawing. Scale bars, 50 μm. B) Microscopy analysis of spheroids in microcapsules after cryopreservation and culture at Day 2 and Day 8. Scale bars, 100 μm.

FIG. 50. Cell viability evaluation of 3D cell structures in microcapsules before and after cryopreservation. The A-549 cells isolated in microcapsules were cultured for 7 days to form spheroids, next microcapsules were cryopreserved in liquid nitrogen, stored for 1 week, recovered and cultivated in cell culture for additional 7 days. A) Microscopy analysis showing microcapsule with spheroids before freezing and after freezing/thawing steps. The cryopreserved spheroids were stored in a liquid nitrogen for 1 week before thawing. Scale bars, 100 μm. B) Cell viability distribution before and after cryopreservation. C) Cell viability as a function of spheroid diameter before and after cryopreservation.

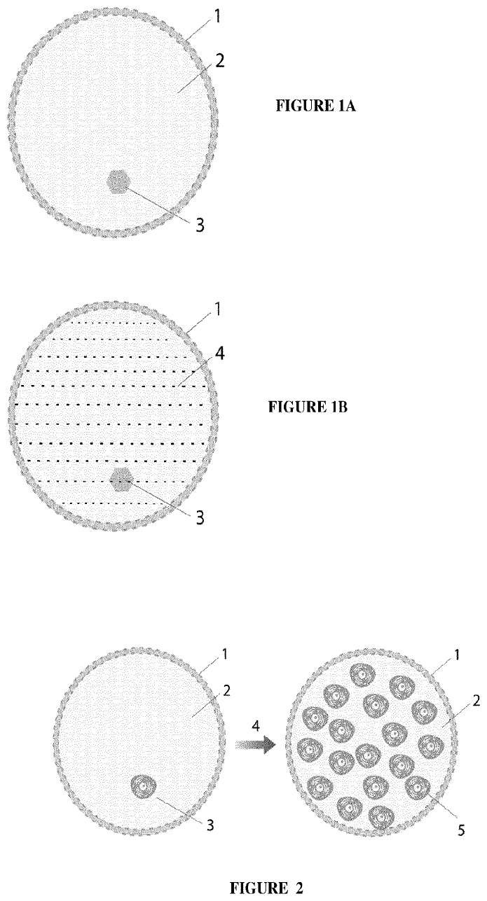

### DETAILED DESCRIPTION OF INVENTION

Throughout the text the terms of “comprising” and “containing” have been used interchangeably and have the same meaning.

The terms “approximately” or “about” are used herein and generally refer to a range of ±30% of the stated value, preferably ±20% of the stated value, more preferably ±10% of the stated value, and even more preferably ±5% of the stated value.

Articles such as “a,” “an,” and “the” include the singular and the plural reference unless the context clearly indicates otherwise. For example, a reference to “a cell” includes one cell and two or more of such cells.

The properties of the microcapsule described below are those at room temperature, i.e. 22° C., unless otherwise specified.

The term “polyampholyte” refers to a polyelectrolyte that bears both cationic and anionic groups, or corresponding ionizable groups, and where the “polyelectrolytes” are polymers whose repeating units bear an electrolyte group. It should be understood that term “polyampholyte” and “ampholytic polymer” are synonyms as defined by IUPAC [41].

### Microcapsules

The present invention provides a microcapsule comprising:
(a) a core comprising a polyhydroxy compound and/or an antichaotropic agent; and
(b) a semi-permeable shell surrounding the core;
wherein the semi-permeable shell comprises a gel formed from a polyampholyte and/or a polyelectrolyte, wherein the polyampholyte and/or the polyelectrolyte in the gel is covalently cross-linked.

In particular, the present invention provides a microcapsule comprising:
(a) a core comprising a polyhydroxy compound and/or an antichaotropic agent; and
(b) a semi-permeable shell surrounding the core;
wherein the semi-permeable shell comprises a gel formed from a polyampholyte, wherein the polyampholyte in the gel is covalently cross-linked.

As is discussed in more detail below, the purposes of the microcapsule include encapsulation of at least one biological entity, i.e. the microcapsule acts as a microcompartment, and may be for isolation, expansion, culture, analysis and/or storage of the at least one biological entity. The purpose of the microcapsule may also be to act as a carrier or a support for the at least one biological entity, where the at least one biological entity is attached to the outer surface of the shell, such as, for example, where the microcapsule provides support for growth of cells in an adherent culture on the outer surface of the shell.

Accordingly, the microcapsule may comprise at least one biological entity. The term “biological entity” includes a cell (including a prokaryotic cell and a eukaryotic cell), a microorganism (including a bacterium, an archaea, or a fungi), a virus, a prion, a nucleus, a chromosome, or a part or product of any of the foregoing (e.g. a cell lysate), and also includes a biological molecule such as a nucleic acid (including a DNA or an RNA) or a protein (such as an enzyme). Preferably the at least one biological entity is at least one selected from a cell, a microorganism, a bacteria, a virus, or a nucleic acid.

The microcapsule may comprise two or more than two biological entities of the same or different type. For example, the microcapsule may comprise a plurality of cells of the same or different cell type and/or subtype. For example, the microcapsule may comprise a plurality of fibroblast cells, alternatively the microcapsule may comprise a population of B- or T-cells of different subtypes. The plurality of cells may form an aggregate or other 3D structure, such as a cell cluster, a spheroid, an organoid, a tumoroid or a tissue.

Where the at least one biological entity is at least one cell, the cell may be a prokaryotic (such as a bacterial cell or an archaea cell) or a eukaryotic cell (such as an animal, plant or fungal cell). In particular, the cell may be a mammalian cell, preferably a human cell. In some examples, the microcapsule may comprise a human cell infected with a virus, a human cell and a bacterial cell, or a bacterial cell infected with a phage.

Where the at least one biological entity is at least one cell, the cell may be a cell that grows in adherent culture, i.e. an adherent cell, or a cell that grows in a suspension.

The at least one biological entity may be in the core of the microcapsule or may be attached to the outer surface of the shell of the microcapsule (e.g. an adherent cell attached to the outer surface of the shell). It is preferred that the at least one biological entity is encapsulated in the core of the microcapsule, such that it is separated from the external environment of the microcapsule by the semi-permeable shell and the microcapsule acts as a microcompartment. Where the at least one biological entity is encapsulated in the core of the microcapsule, it may be e.g. suspended in a liquid core, or located at an inner surface of the semi-permeable shell (e.g. at the interface between the shell and the core). In particular, where the microcapsule acts as a microcompartment for at least one adherent cell the cell may be attached to the inner surface of the shell. This would enable it to grow as an adherent culture within the microcapsule.

In addition, or alternatively, to the at least one biological entity, the microcapsule may comprise at least one solid particle, optionally wherein the at least one solid particle is a metal nanoparticle, a mineral particle, a polymer particle, a fluorescent nanoparticle, a magnetic nanoparticle or a composite particle. These particles may carry ligands or other functional groups, e.g. DNA primers, reagents, antigens, etc., for use in the methods of culturing and analysis described herein. For example, a microcapsule may comprise a magnetic or polymer particle attached to an antigen for use in a sandwich ELISA assay to be performed in the microcapsule. The particle may be present in the shell and/or the core of the microcapsule. The size of said particle is preferentially from 10 nm to 10 μm, and can be chosen depending on the intended use and the size of the microcapsule.

### The Shell

The microcapsules described herein comprise a core surrounded by a semi-permeable shell, (which in most embodiments is a single-layer shell with no additional layers between the core and the shell). The semi-permeable shell permits the passive diffusion (down a concentration gradient) of lower molecular weight molecules and compounds, while retaining larger molecular weight molecules, particularly the biological entity which is to be kept encapsulated/compartmentalized, within the microcapsule. Accordingly, the permeability/porosity of the shell of the microcapsule should be selected according to the purpose to which the microcapsule is to be put. For example, where the microcapsule is to be used for cell culture the lower molecular weight molecules diffusing through the shell, (i.e. between the external environment of the microcapsule and the core) will be cell culture and/or storage medium(s). If the microcapsule is to be used for nucleic acid analysis the lower molecular weight molecules may be a polymerase, a reverse transcriptase enzyme, primers and/or other reagents and substrates. It may also be desirable to ensure that the permeability ensures that e.g. waste products from certain reactions (e.g. products from cell lysis), or products from cell culture, can diffuse across the shell from the core to the external environment). The permeability/porosity should also be selected bearing in mind the desired time frame in which diffusion should occur; diffusion of molecules may take longer where their size is very close to the pore size of the shell.

As is described further herein, microcapsules having different shell permeabilities (porosities) can be prepared using the method of the invention by adjusting the concentration of the shell precursors (i.e. the polyampholyte and/or polyelectrolyte) that are used to form the shell, adjusting the number of cross-linking moieties, and/or altering the composition of the shell with additives.

In some examples the biological entity may be a cell that is encapsulated for the purpose of cell culture. In such examples, there is a significant difference between the relatively large biological entity being retained in the microcapsule and the relatively small molecular weight of the cell culture reagents that need to be permitted to diffuse into the microcapsule and cell waste products that need to be permitted to diffuse out of the microcapsule during use. In such examples, any microcapsule selected from a group having a broad range of shell permeabilities can be used (although it may also be desired to ensure that certain cell products can diffuse from the core to the external environment). In other examples, where the molecular weight of the biological entity to be retained within the microcapsule is much lower than that of a cell, the distinction in terms of molecular weight between the biological entity and the largest of the compounds that are to be permitted diffuse into and out of the microcapsule is narrower, and the requirements for the permeability of the shell are stricter. Such examples include those where the biological entity is a polynucleotide of at least 100 nucleotides in length and the microcapsule is to be used as a microcompartment in which to perform reactions (e.g. amplification, labelling etc) on the polynucleotide. In such examples, the polynucleotide is retained in the core while enzymes, such as reverse transcriptase and nucleic acid polymerase that may have molecular weights of about 100,000 Da or less, and/or antibodies that may have molecular weights of about 150,000 Da, and/or primers and oligonucleotides that may about 30-100 nucleotides in length or less, can diffuse between the external environment and the core of the microcapsule.

In some examples, of the invention the semipermeable shell of the microcapsule retains an at least one biological entity which is a cell in the core of the microcapsule, while permitting regents of cell culture and products of cell culture to diffuse to and from the core.

In some examples of the invention the semipermeable shell of the microcapsule is impermeable to nucleic acids larger than 200 nucleotides, 150 nucleotides, or preferably 100 nucleotides in length, while being permeable to nucleic acids shorter than 100 nucleotides, preferably shorter than 50 nucleotides.

In some examples, the semi-permeable shell allows for diffusion of smaller molecular weight compounds having a molecular weight of 120,000±80,000 Da or less through the shell, while retaining larger molecular weight compounds having molecular weight of 300,000±100,000 Da and above. (In particular examples, the microcapsules can contain very large molecular weight compounds including a cell genome, which has a mass of 2.15×10\hat{ }9 Da.)

In some examples, the said microcapsule's shell is permeable to low molecular weight molecules and compounds that may diffuse into and out of the core of the microcapsule. For example, microcapsule's shell may be permeable to compounds, reagents, molecules having molecular weight smaller than approximately 1000 Da, 2000 Da, 3000 Da, 5000 Da, 10,000 Da, 20,000 Da, 30,000 Da, 50,000 Da, 100,000 Da, 120,000 Da, 200,000 Da, 300,000 Da, 400,000 Da or 500,000 Da.

Alternatively, or in addition, the said microcapsule's shell prevents larger biomolecules from entering, or leaving, the core of the microcapsule. For example, microcapsule's shell may prevent biochemical compounds, reagents and molecules having molecular weight larger than approximately 10,000 Da, 20,000 Da, 30,000 Da, 50,000 Da, 100,000 Da, 200,000 Da, 300,000 Da, 500,000 Da or 1,000,000 Da from entering and leaving the core of the microcapsule.

As noted above, the semi-permeable shell of the microcapsule is formed from a polyampholyte and/or a polyelectrolyte, and preferably may be formed from a polyampholyte or a composite mixture of polyampholyte and polyelectrolyte. The polyampholyte and/or the polyelectrolyte in the gel are covalently cross-linked. Specifically, the individual polymer strands of the polyampholyte and/or the polyelectrolyte are cross-linked to each other to create a polymer mesh, i.e. the cross-links comprise intermolecular cross-links, and form an elastic gel.

Polyampholytes are a class of natural and synthetic polymers that comprise neutral, positively and negatively charged groups, and are thus soluble in aqueous solution. Polyampholytes offer a unique set of properties defined by the interactions between the charged groups (e.g., interaction between amino acid side chains). Polyampholyte gels are strongly viscoelastic, have high toughness, high fatigue resistance, tunable mechanical properties, supports swelling due to changes in pH or salt concentration [42, 43]. As revealed below, the invention disclosed here benefits from the physicochemical properties of polyampholyte gels.

In some examples, the polyampholytes and/or polyelectrolytes are “thermo-responsive” polymers. As is discussed further below, “thermo responsive” polymers are those that are capable of undergoing a transformation when subjected to a change in temperature. In the context of the present invention the “thermo responsive” polymers are capable of forming a gel when subjected to a change in temperature, for example when cooled, below sol-gel transition temperature. The gel that is formed in response to the temperature change is a mesh or 3-dimensional network of polymer strands, with a solid structure due to physical cross-linking of individual polymer strands.

The polyampholyte and/or the polyelectrolyte may be a natural biopolymer, a modified biopolymer or a synthetic polymer.

In a preferred example, the polyampholyte and/or the polyelectrolyte comprise peptide bonds, such that the semi-permeable shell is hydrolysable with a protease. In particular, this is advantageous as it enables the shell of the microcapsule to be broken using a protease enzyme to release the inner content, including any biological entity, comprised in the microcapsule. Accordingly, the harsher chemical conditions that may be necessary to break some of the microcapsules of the prior art (e.g. those formed with a shell comprising PEG) are avoided, and also the risk of damage to the encapsulated biological entity can be reduced.

In particular, the polyampholyte and/or the polyelectrolyte comprise amino acids and is a peptide, a polypeptide, an oligopeptide or a protein. Accordingly, the polyampholyte and/or the polyelectrolyte may be described as “proteinaceous”. The proteinaceous polyampholytes that show liquid-liquid phase separation properties are typically characterized by long segments of low diversity amino acids. These segments are often repetitive and are enriched in glycine (G), glutamine (Q), asparagine (N), serine (S), arginine (R), lysine (K), aspartate (D), glutamate (E) or aromatic amino acids such as phenylalanine (F) and tyrosine (Y) amino acids. These segments often encompass multiple short motifs such as YG/S-, FG-, RG-, GY-, KSPEA-, SY- and Q/N-rich regions, or regions of alternating charges [44].

As is described further below, the microcapsules of the present invention can be made by creating a droplet comprising a first solution of the polyampholyte and/or polyelectrolyte, and a second solution of the polyhydroxy compound and/or the antichaotropic agent of the core. Therefore, it is important that the polyampholyte and/or the polyelectrolyte are polymers that able to undergo liquid-liquid phase separation in the droplet. Numerical simulations and experimental evidence have shown that polyampholytes such as intrinsically disordered proteins (IDPs), elastin-like polypeptides (ELP), proteins comprising structured and disordered regions, and variety of synthetic and natural biopolymers may self-assembly into coacervates and form polyampholyte-rich liquid phase, and a polyampholyte-dilute liquid phase [45-49]. The IDPs often comprise highly repetitive and low complexity amino acid sequences, and contain disorder-promoting amino acids such as glycine and/or proline, and may also contain glutamate, serine, lysine, alanine, arginine and/or glutamine [50, 51].

Accordingly, in some embodiments of this invention the polyampholyte is a protein, polypeptides or oligopeptides with a primary amino acid sequence comprises at least 10% disorder promoting amino acids, and preferably at least 30%. Disorder promoting amino acids include proline, glycine, glutamic acid/glutamate, serine, lysine, alanine, arginine, and glutamine.

A significant fraction of extra-cellular proteins is expected to be enriched in disorder promoting amino acids (e.g., proline and/or glycine) and thus may be used for making a disclosed microcapsule. One specific example non-limiting example is collagen, the polyampholyte enriched in disorder promoting amino acids, proline and glycine. In some specific cases microcapsules may be created whose shell is made of proteins, polypeptides or oligopeptides containing disordered segment of ≥30 amino acid long, where the term “disordered segment” means the amino acid sequence does not adopt any tertiary structure and may comprise disorder-promoting amino acids.

Another, highly relevant class of polyampholytes that may be used for making a disclosed microcapsule are the elastin-like polypeptides (ELPs). The ELPs share a common amino acid sequence (Valine-Proline-Glycine-X-Glycine)n, or close analogues such as (Valine-Proline-Alanine-X-Glycine)n or (Isoleucine-Proline-Glycine-X-Glycine)n, where n is the monomeric unit, and where the “X” denotes any amino acid. The amino acid in a position “X” affect the coacervation temperature and other biochemical properties of the ELPs. As such choosing the corresponding amino acid in a position “X”, it is possible to alter the physical and biochemical properties of the ELP gel such as gelation temperature, the elasticity of the hydrogel, Young modulus, etc. It has been elucidated that the length of charged blocks, block asymmetry, and charge asymmetry, and number of connections between oppositely charged blocks in polyampholytes are critical for liquid-liquid phase separation [52]. The ELPs include various proteins and polypeptides such as elastin, gluten, gliadin, abductin, resilin, tropoelastin, fibronectin, silks proteins that may be suitable for making a disclosed microcapsule. Therefore, in the context of this invention the aforementioned studies can provide some guiding principles for rationally designing the polyampholytes for semi-permeable microcapsule synthesis.

In view of the above, the polyampholyte may be an extracellular matrix protein, a proteoglycan, a glycosaminoglycan, or a hydrolyzed form of any of the foregoing. Preferably, the polyampholyte is selected from the group consisting of collagen, mucin, laminin, elastin, elastin-like polypeptides, fibrin, silk fibrion, fibronectin, vimentin, or hydrolyzed forms thereof, such as gelatin. In a particularly preferred embodiment the polyampholyte is gelatin or a derivative thereof.

Alternatively, the polyampholyte may be a product such as Matrigel™ or Geltrex™, or synthetic analogs thereof.

The structure of the semi-permeable shell is further stabilized by covalent cross-linking between the polymer strands of the polyampholyte and/or the polyelectrolyte. Such covalent cross-links may be formed with groups that are part of the polyampholyte and/or the polyelectrolyte. For example, when the polyampholyte comprises amino acids the amine groups can be covalently cross-linked using glutaraldehyde or genipin. Alternatively, the polyampholyte and/or the polyelectrolyte may be modified with a chemical group, which chemical group participates in a covalent cross-linking reaction to form the covalent cross-link. Examples of suitable chemical groups are acrydite, acrylate, methacryloyl, acrylamide, methacrylamide, bisacrylamide, methacrylate, methacrylic acid, acrylic acid, polyacrylic acid, methacrylic anhydride, acryloyl, vinyl, vinylsulfone, vinylpyrrolidone, thiol, disulphide, cystamine, carboxyl, amine, imine, azide, triazole, tetrazine, azidophenylalanine, alkynyl, alkenyl, alkynes, diisocyanate, hydroxypropionic acid, hydroxy phenol, azobenzene, methylcyclopropene, trans-cyclooctene (TCO), norbornene, diacrylcyclooctyne (DBCO) or cyclooctanyl moieties and/or reagents. Further suitable chemical groups are set out in the section regarding the “Production of the Microcapsule” below. Preferred chemical groups are methacryloyl, methacrylamide or methacrylate.

Accordingly, in some examples of the microcapsule, the polyampholyte from which the gel is formed is modified with a chemical group and is a gelatin derivative, preferably selected from gelatin methacryloyl, gelatin methacrylamide, gelatin acrylamide and gelatin methacrylate. Preferably the gelatin derivative is gelatin methyacrylate.

The degree of substitution of these chemical groups on the polyampholyte and/or polyelectrolyte can be varied to achieve the desired microcapsule stability, which will depend on the nature of the reactions that are to be performed on the biological entity in the microcapsule and/or the processes to which the microcapsule is to be subjected. For example, the polyampholyte, such as gelatin derivatives, may have a degree of substitution of 10 to 90%, or 20 to 90%, preferably 40 to 90%, and more preferably 60 to 80%.

The shell may be formed from other precursors (additives) in addition to the polyampholyte and/or the polyelectrolyte and/or comprise more than one type of polyampholyte and/or polyelectrolyte. In particular, the microcapsule's shell may comprise a composite mixture and/or include synthetic polymers (e.g., PEG, poly-L-lysine) that may change the properties of the shell (e.g., porosity, stiffness, elasticity, mechanical stability, etc.). Proteinaceous material does not need to be the major ingredient or exclusive precursor of the cross-linked shell.

### The Core

The core of the microcapsule may comprise polyhydroxy compound and/or an antichaotropic agent. The polyhydroxy compound may be a naturally occurring polymer or derivatives thereof. In particular, the polyhydroxy compound may be selected from a polysaccharide, a carbohydrate, an oligosaccharide, or a sugar. In one example, the polyhydroxy compound is one or more of dextran, alginate, hyaluronic acid, glucan, glycogen, starch (amylose, amylopectin), agarose, agar-agar, heparin, pectin, cellulose (including hydroxyethyl cellulose), hemicellulose, chitosan, chitin, xanthan gum, curdian, pullulan, inulin, graminan, levan, carrageenan, polyglycerol, and derivatives of the foregoing that are chemically modified or partly hydrolyzed. Preferably the polyhydroxy compound is glucan, more preferably dextran. Alternatively, the polyhydroxy compound may be a synthetic polymer, such as Ficoll (e.g. Ficoll PM 4000).

The polyhydroxy compound is preferably an enzyme degradable polymer, such that it can be hydrolyzed upon treatment with a hydrolase (e.g. a glycosidase, a dextranase, an amylase, or a cellulase). This is advantageous since, as described further below, it enables the viscosity of the core of the microcapsule to be reduced without breaking the microcapsule. In addition, it further allows the polyhydroxy compound to be more effectively separated from the biological entity in the core, when the biological entity is released when the microcapsule is broken.

The polyhydroxy compound may have a molecular weight of 300 Da to 5000 kDa. In one example the molecular weight is greater than 10 kDa (i.e. is between 10 kDa and 800 kDa).

In another example the molecular weight is greater than 100 kDa (i.e. is between 100 kDa and 800 kDa). In a preferred example, the polyhydroxy compound has a molecular weight of 400 to 600 kDa, more preferably approximately 500 kDa.

The antichaotropic agent may be kosmotropic salt, and in particular may be a carbonate, a sulphate, a phosphate or a citrate. In a preferred example kosmotropic salt is an ammonium sulphate.

The core of the microcapsule may be liquid, semi-liquid or a hydrogel. In particular, the hydrogel may be formed during the production of the microcapsule, as is described further below.

As noted above, due to the semi-permeable nature of the shell, low molecular weight molecules and compounds can be removed from the core by placing the microcapsule in a suitable external environment to set up a concentration gradient to allow the low molecular weight molecules and compounds to passively diffuse from the core down the concentration gradient to the external environment. Similarly, low molecular weight molecules and compounds can passively diffuse into the microcapsule from the external environment. In this manner the composition of the core of the microcapsule can be altered. In particular, where the core comprises a polyhydroxy compound of a relatively high molecular weight (which do not diffuse across the shell) this compound can be hydrolysed as described above to produce low molecular weight hydrolysis products that can diffuse across the shell. Alternatively, the polyhydroxy compound used to form the microcapsule may have a relatively low molecular weight, and depending on the permeability of the shell of the final microcapsule, may diffuse out of the microcapsule when the final microcapsule is placed in a suitable external environment.

Accordingly, the invention also provides a microcapsule in which the core no longer comprises a polyhydroxy compound and/or an antichaotropic agent which were present when the microcapsule was produced. Particular aspects are:
(i) A microcapsule comprising:
   (a) a core comprising a reaction buffer, a cell culture medium or a cell storage buffer; and
   (b) a semi-permeable shell surrounding the core;
   wherein the semi-permeable shell comprises a gel formed from a polyampholyte and/or a polyelectrolyte, wherein the polyampholyte and/or the polyelectrolyte in the gel is covalently cross-linked;
(ii) A microcapsule comprising:
   (a) a core comprising a reaction buffer, a cell culture medium and/or a cell storage buffer; and
   (b) a semi-permeable shell surrounding the core;
   wherein the semi-permeable shell comprises a gel formed from a polyampholyte, wherein the polyampholyte in the gel is covalently cross-linked.

As above, preferably such microcapsules comprise the at least one biological entity.

### Microcapsule Properties

The examples of the present disclosure show that microcapsules with a range of sizes and shell thicknesses can be made, including at sizes above 60 μm in diameter. In particular, the examples provided below demonstrate that the core and shell material described herein reliably produce microcapsules encapsulating cells, even those of larger size. Microcapsule dimensions can be measured from images taken with a microscope.

In particular, the microcapsule may be from about 1 μm to about 100,000 μm in diameter, from about 1 μm to about 10,000 μm in diameter, from about 1 μm to about 1,000 μm in diameter, from about 1 μm to about 500 μm in diameter, from about 20 μm to about 200 μm in diameter, from about 60 μm to about 150 μm in diameter. The size of the microcapsule can be selected according to the use to which the microcapsule is to be put. For the encapsulation and analysis of a mammalian cell, a microcapsule of from about 60 μm to about 150 μm in diameter may be suitable. For cell culture to produce a 3D cell assembly such as a spheroid, larger microcapsules can be selected. The diameter referred to is generally the largest diameter of the microcapsule, although in many embodiments the microcapsules are generally circular.

The shell of the microcapsule may be from about 0.2 μm to about 100 μm thick, preferable about 1 μm to about 10 μm thick. Again, the thickness can be selected according to the use to which the microcapsule is to be put. For analysis of a single cell, a thickness in the range of 2 to 6 μm, and preferably about 5 μm can be used. The thickness referred to is generally the maximum thickness of the shell. Although for most embodiments the shell thickness is generally uniform (e.g. with the thickness of the thinnest part of the shell being no more than 10% less than the thickness of the thickest part of the shell).

Another advantage of present disclosure is that the microcapsule may comprise a very thin shell (1-4 μm thick) and still support mechanical integrity of the microcapsule and retain encapsulated cell and/or 3D cell assemblies. The microcapsules disclosed here maintain integrity even when the shell is thin (below 5 μm) and the radius of the microcapsule is large (>100 μm). The use of thin shell may, in some cases, be important for facilitating the diffusion of growth factors and other biochemical compounds from the exterior environment to the core of microcapsule. Moreover, the high elasticity of the cross-linked shell prevents the microcapsules from bursting during cell growth and 3D cell assembly formation.

In one example the semi-permeable shell and the core are concentric or approximately concentric.

In particular, one of the unique advantages of present disclosure is that microcapsule has high circularity and high concentricity. Considering the average radius, R, of the microcapsule being R=√(square root over (S/π)), wherein S is the equatorial transverse surface of the capsule. The circularity, C, is a ratio of the minor axis (R min) over the major axis (R max) of the ellipse adjusted to the external edge of the projected equatorial section. In the present disclosure the microcapsule may have a circularity C=0.8±0.2, preferably C=0.9±0.1, more preferably C=0.94±0.06, and even more preferably C=0.95±0.05.

The concentricity, O, of the microcapsule is defined as O=(Wmin/Wmax)*100%, wherein Wmin is thinnest part of the shell and Wmax is the thickest part of the shell. In the present disclosure the microcapsule shows O≥66%. The high circularity and concentricity of microcapsules may be advantageous when culture of encapsulated cells in plurality of microcapsules requires identical conditions. For example, if microcapsule comprises uneven shell the diffusion of nutrients could be affected with highest flux of nutrients through the thinnest part of the shell. Also, poor circularity could affect the structure of 3D cell assemblies produced inside the microcapsule. In addition, in some examples high circularity and concentricity may be important during the performance of reactions in the microcapsule to analyze the at least one biological entity comprised in the microcapsule, e.g. a nucleic acid, to ensure that reactions are efficient. However, it should be understood that in other examples the microcapsule may be of irregular shape too, such as having oval, oblong, amorphous, pancake, cylindrical, or non-spherical shape.

As indicated above, the microcapsules of the present invention are robust, and their chemical and physical stability gives the microcapsules a wide range of use.

In particular, the microcapsules are thermostable and can withstand heating.

Accordingly, the microcapsules can be used for methods comprising steps including thermocycling (PCR), and/or incubation at elevated temperatures, such as for protein denaturation. In particular, the microcapsules do not disintegrate on incubation at an elevated temperature (such as 10 minutes at 95° C.). As a result, after such incubation the microcapsule continues to retain its core/shell structure and any biological entity comprised in the core is not lost.

The microcapsule is stable, i.e. retains its shell and core structure, in standard cell culture conditions for at least 2 weeks, preferably at least one month (provided the growth of cells does not rupture the semi-permeable shell).

It will be appreciated that the dimensions of the microcapsule described in the paragraphs above refer to the size and shape of the microcapsule after production. However, due to the elastic nature of the shell, the microcapsule is suitable for growth of cells in the core. In particular, in some embodiments the volume of the microcapsule can increase at least 2-times, or at least 4-times. With thinner shells of between 2 to 10 μm in thickness (e.g. of about 3-4 μm) the volume of the microcapsule may be increase at least 8-times without rupture as the cells inside the core proliferate and stretch the shell.

In a further aspect the present invention provides a plurality of microcapsules, the microcapsules being as defined herein. In particular, the method of producing the microcapsules that is discussed further below, generally produces a plurality of microcapsules.

The plurality of microcapsules may be monodisperse, or polydisperse, and be about 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, 5 mm, 10 mm or even 100 mm in size. In some cases, a microcapsule may have a diameter of at least about 1 μm, 5 μm, 10 μm, 20 μm, 30 μm, 40 μm, 50 μm, 60 μm, 65 μm, 70 μm, 75 μm, 80 μm, 90 μm, 100 μm, 250 μm, 500 μm, 1 mm, 5 mm, 10 mm or even 100 mm in size, or more. In some cases, the size of a microcapsule may vary and be in range of about 1-100 μm, 10-100 μm, 1-1000 μm, 10-1000 μm, 0.1-10 mm, 1-10 mm or 1-100 mm.

The microcapsule or the plurality of microcapsules of the invention may be comprised in a composition with a carrier oil or an aqueous solution. Suitable carrier oils are described further below. The aqueous solution may be a reaction buffer, or a buffer for washing, transporting or storing the microcapsules. In particular, where the microcapsule(s) comprise at least one cell, the aqueous buffer may be a buffer for washing cells, such as PBS or HEPES, or may be a storage buffer comprising a cryoprotectant.

Further features of the polyampholyte and/or the polyelectrolyte of the shell, the polyhydroxy compound and/or the antichaotropic agent of the core, and the properties of the microcapsule are described in the following section, which sets out the process for producing the microcapsule, but are equally applicable to the microcapsule per se as described in this section.

### Process for Producing the Microcapsules

The microcapsules of the invention may be prepared by a method comprising:
(a) forming a droplet from a first solute and a second solute, wherein the first solute is a polyampholyte and/or a polyelectrolyte and the second solute is a polyhydroxy compound and/or an antichaotropic agent, wherein the polyampholyte/polyelectrolye comprise one or more covalently cross-linkable groups;
(b) allowing phase separation inside the droplet into a shell phase (outer film) enriched in the first solute and a core phase enriched in the second solute, and gelation and/or precipitation in the shell phase to form an intermediate microcapsule;
(c) forming intermolecular covalent cross-links with the one or more covalently cross-linkable groups to form a microcapsule comprising a semi-permeable shell of covalently cross-linked polyampholyte and/or polyelectrolyte and a core.

In particular, where the microcapsule is to comprise at least one biological entity the method may be a method of encapsulating at least one biological entity, or a method of producing a microcapsule encapsulating at least one biological entity, the method comprising:
(a) isolating the at least one biological entity in a droplet with a first solute and a second solute, wherein the first solute is a polyampholyte and/or a polyelectrolyte and the second solute is a polyhydroxy compound and/or an antichaotropic agent, wherein the polyampholyte/polyelectrolyte comprises one or more covalently cross-linkable groups;
(b) allowing phase separation inside the droplet into a shell phase (outer film) enriched in the first solute and a core phase enriched in the second solute, and gelation and/or precipitation in the shell phase to form an intermediate microcapsule; and
(c) forming intermolecular covalent cross-links with the one or more covalently cross-linkable groups to form a microcapsule comprising a semi-permeable shell of covalently cross-linked polyampholyte or polyelectrolyte and a core, wherein the biological entity is in the core.

It is noted that, depending on conditions and the nature of the first and second solutes, phase separation, and gelation and/or precipitation may begin to occur as soon as the droplet forms. Alternatively, phase separation may occur first, followed by gelation and/or precipitation Accordingly, these processes may occur (essentially) simultaneously or separately.

### Forming the Droplets

The droplet may be a water-in-oil droplet, a water-in-water droplet or a water-in-air droplet. In a preferred example the droplet is a water-in-oil droplet.

The droplet may be produced in any suitable device. In particular (a) may be performed in a microfluidic device or other device, assembly or instrument capable of forming a droplet, such as a glass capillary device. In a preferred example, a microfluidic device is used.

The process of producing the microcapsule may involve the (co)encapsulation of, i) the first solute, ii) the second solute, and optionally iii) the at least one biological entity (e.g. cell), in a droplet. Generally, a plurality of droplets is produced. The first solute, the second solute, and optionally the biological entities (e.g. cells), may be added to the same aqueous solution or to separate aqueous solutions and emulsified with a continuous phase (e.g. carrier oil, or an aqueous medium) having a surfactant (preferably a non-ionic surfactant), to generate water-in-oil (when continuous phase is immiscible oil) or water-in-water droplets (when continuous phase is an aqueous medium), respectively, or sprayed into air to form the water-in-air droplets. For example, a first aqueous solution comprising the first solute may be emulsified along with a second aqueous solution comprising second solute and optionally the biological entity, to create droplets. As another example, an aqueous solution comprising the first solute may be emulsified along with multiple aqueous solutions comprising the second solute and/or the biological entities to create droplets. In particular, where the at least one biological entity is two or more types of biological entity, these may be comprised in the same or separate aqueous solutions prior to mixing. Other emulsification strategies will be known to a person experienced in the art. It should be noted that the solutions may comprise further components which are to be incorporated in the shell or the core.

By controlling the volumetric ratio of aqueous solutions loaded into the droplets (e.g. by using different infusion rates) the said droplets may comprise a desirable ratio of first solute, second solute, and optionally the at least one biological entity. Emulsification can be performed using extrusion, shaking, agitation, micro-sieve, microfluidics system, glass capillary assemblies or other droplet generation devices and/or methods. Droplets may be generated in so called dripping mode or in so called jetting mode. Water-in-oil droplets may be either monodisperse or polydisperse, whereas a more preferable case is monodisperse droplets.

In particular, water-in-oil droplet generation methods are well described and are known to the skilled person in the art [53-61] including (Torii et al., JP Pub. No. 2004/083802; Link et al., WO 2004/091763; Weitz et al., U.S. Pub. No. 2009/0012187; Bibette et al., WO 2010/063937; Weitz et al. U.S. Pub. No. 2012/0211084; Weitz et al., U.S. Pub. No. 2013/0064862). In a preferred case scenario, an emulsion comprising water-in-oil droplets may be generated using a microfluidic device.

The water-in-oil droplets may be formed in a fluorinated, perfluorinated, hydrocarbon or synthetic continuous oil phase. The water-in-oil droplets may be stabilized with fluorosurfactants, for example based on Krytox and PEG co-polymers [62], supplemented in the carrier oil.

Further, water-in-water droplet generation methods are well described and are known to the skilled person in the art [63]

In a preferred case scenario the water-in-oil droplets are generated in a microfluidic device having a flow focusing junction [57]. A microfluidic device may contain microchannels of different lengths and/or widths and/or heights that intersect at a junction (e.g., nozzle, flow focusing junction) where the aqueous phase gets dispersed in the continuous (carrier oil) phase.

More than one aqueous phase may be introduced separately in a microfluidics device and brought into contact just upstream of the nozzle or at the nozzle, or downstream the nozzle. For example, one aqueous phase containing the first solute may be brought in contact with a second aqueous phase containing the second solute and optionally the at least one biological entity, and then brought into contact with the carrier oil. When one or more aqueous phases meet the carrier oil the water-in-oil droplets may form, for example, at the flow focusing junction, or downstream the flow focusing junction.

Droplets may be of different size, ranging from 10 μm to 100 mm, and more preferably in the range of 50-1000 μm. For example, where the droplet is a water-in-oil droplet, by controlling the size of the microfluidics channels, the geometry of the flow focusing junction and the speed at which the aqueous phase(s) and the carrier oil are introduced into a microfluidics chip, the droplet size may be precisely controlled.

In one example, the droplet may be produced using a microfluidics system comprising:
(i) an inlet and a microfluidic channel for the continuous oil phase;
(ii) an inlet and a microfluidic channel for a first solution comprising the first solute;
(iii) an inlet and a microfluidic channel for a second solution comprising the second solute;
(iv) a nozzle where carrier oil meets the aqueous solutions;
(v) a water-in-oil droplet collection outlet; and
(vi) a microfluidic channel connecting the flow focusing junction with the outlet.

The microfluidic system may optionally comprise one or more inlet(s) and microfluidic channel(s) for other aqueous solution(s).

In another example, the water-in-oil droplets may be produced using a microfluidics system comprising:
one or several inlets for the continuous phase (carrier oil) optionally supplemented with a surfactant;
one or several inlets for the first aqueous solution comprising the first solute;
one or several inlets for the second and any additional aqueous solution(s) comprising the second solute and/or the at least one biological entity;
a microchannel where the aqueous fluids are combined;
the flow focusing junction (nozzle) where carrier oil meets the aqueous solutions(s);
the channel for droplet stabilization by the surfactant supplemented in the carrier oil, and;
the water-in-oil droplet collection outlet.

The first solution may comprise 0.1 to 20% (w/v) of the polyampholyte and/or polyelectrolyte, optionally 1 to 15% (w/v) of the polyampholyte. In the preferred embodiment where the polyampholyte is a gelatin derivative selected from gelatin methacryloyl, gelatin methacrylamide, gelatin acrylamide and gelatin methacrylate, and the first solution may comprise in the range of 2 to 6% (w/v) of the gelatin derivative.

In examples where the polyampholyte is modified with one or more chemically cross-linkable groups for the covalent cross-linking in (c), the polyampholyte may have a degree of substitution of 10 to 90%, optionally 40 to 90%. In a preferred embodiment where the polyampholyte is the gelatin derivative of the above paragraph, the degree of substitution may be 60 to 80%.

The second solution may comprise 0.1 to 40% (w/v) of the polyhydroxy compound. In particular, in the preferred embodiment where the polyhydroxy compound is dextran the second solution may comprise 5 to 30% (w/v) of the dextran. Alternatively, the polyhydroxy compound may be a synthetic polymer such as Ficoll and accordingly the second solution may comprise 3 to 30% (w/v) of the Ficoll.

In addition, or alternatively, the second solution may comprise an antichaotropic agent at a concentration of about 0.1 to about 2 M, preferably about 0.5 to about 1.5 M.

### Liquid-Liquid and Liquid-Solid Phase Separation

Step (b) of the method comprises allowing phase separation inside the droplet into a shell phase (outer film) enriched in the first solute and a core phase enriched in the second solute, and allowing gelation and/or precipitation in the shell phase to form an intermediate microcapsule.

The skilled in the art will be aware of liquid-liquid phase separation phenomena in aqueous two phase systems [64, 65], which may occurs in aqueous solutions comprising different water-soluble polymers, or an aqueous mixtures comprising a single polymer and a certain salt. The skilled in the art will be also aware of liquid-solid phase separation phenomena often known as precipitation [66]. Similar to liquid-liquid phase separation, liquid-solid phase separation typically arises during desolvation, when favorable intermolecular interactions between the polymers arises, which then leads to abrupt expulsion of counterions and water. Ability to distinguish between liquid-liquid and liquid-solid phase transitions is not a trivial task and classical techniques such as turbidimetry are not considered to be suitable [66]. Consequently, the terms liquid-liquid phase separation (coacervation) and liquid-solid phase separation (precipitation) are sometimes used interchangeably. Liquid-liquid and liquid-solid phase separation may also occur in aqueous solutions comprising a macromolecule (e.g. biopolymer) in response to temperature change, salt concentration or pH change. Liquid-liquid phase separation may also occur when two, or more, polymers such as oppositely charged polyelectrolytes (e.g., a mixture of oppositely charged proteins such as poly-lysine and poly-glutamic acid), interact with each other and form a condense phase enriched in both polyelectrolytes. Liquid-liquid phase separation may also be described through a coacervation [67], a thermodynamic process when macromolecules (such as biopolymers, polyelectrolytes or polyampholytes) in aqueous solution undergo liquid-liquid phase separation [68-70]. When right conditions are met the macromolecules may form a dense phase in a thermodynamic equilibrium with a dilute phase, where the dense phase comprising the macromolecule may be referred as coacervate. The relative interaction strength (e.g., ionic and/or hydrophobic) between the macromolecules involved in coacervate formation, temperature, pH, salt concentration, and the chemical groups on the macromolecules, contribute to the saturating concentration at which coacervation takes place and coacervates are produced [71]. Physical and chemical factors driving coacervate formation via liquid-liquid phase separation have been extensively studied [71-74]. Likewise, physical and chemical factors driving liquid-solid phase separation have been reported [66, 75].

In the context of this invention liquid-liquid phase separation, liquid-solid phase separation, or a combination of both, may be applied to generate a microcapsule. However, it should be understood that the generation and use of microcapsule does not depend on a specific type of phase separation that occurs during the microcapsule generation/production process.

The liquid-liquid phase separation or liquid-solid phase separation may be performed in bulk. The liquid-liquid phase separation or liquid-solid phase separation may be performed in emulsion droplets. In a preferred case the liquid-liquid or liquid-solid phase separation phase separation is conducted in water-in-oil droplets, or water-in-water droplets. Person experienced in the field will be aware of the techniques to generate water-in-oil or water-in-water droplets that are also described above. It may be desirable to produce monodisperse water-in-oil droplets. Encapsulation of polyampholyte along with other macromolecules (e.g., polyhydroxy compound) and/or antichaotropic agent (e.g., kosmotropic salts) may be used control and organize the liquid-liquid phase separation or liquid-solid phase separation inside the liquid droplets, and formation of the core/shell structure.

### Allowing Phase Separation—Liquid-Liquid and Liquid-Solid Phase Separation Inside Water-In-Oil Droplets

Throughout the text of this section and below the terms of monomer and precursor have been used interchangeably and have the same meaning. Likewise, solidification and gelation have been used interchangeably and have the same meaning.

In the context of this disclosure, it is preferable to achieve conditions when two co-existing aqueous phases, emulsified in droplets (preferably water-in-oil droplets), preferentially phase separate into an outer shell phase (liquid shell) and an inner phase (liquid core). Liquid-liquid phase separation may occur upon a difference of two-aqueous phases in solvent affinity, sufficient to induce phase separation. Liquid-liquid phase separation may be enhanced by irradiation, temperature, salts, favorable interactions, coacervation, pH change, enzymatic or chemical treatment, or any combination thereof. When liquid-liquid phase separation occurs, the liquid shell (liquid film) must totally envelope the liquid core. Otherwise the microcapsules will be defective. When the liquid film is gelled, an intermediate microcapsule is formed, which is discussed further below. In some circumstances, when the liquid film is gelled, the liquid core may also form a hydrogel. In such case the intermediate microcapsule having a hydrogel core is formed.

In some circumstances, it may be also preferable to achieve the conditions when macromolecules acting as precursors of the microcapsule's shell (including the polyampholyte and/or the polyelectrolye) preferentially phase separate into a solidified outer phase (shell) contemporaneously, during the inner phase (core) formation. This may occur when gelled (outer) phase and liquid (inner) phase experience difference in solvent affinity sufficient to induce liquid-solid (or liquid-gel) phase separation. Formation of solidified outer phase may be promoted by irradiation, temperature, salts, favorable interactions, coacervation, pH change, enzymatic or chemical treatment, or any combination thereof. When liquid-solid phase separation occurs, the solidified shell must totally envelope the liquid core. In some circumstances, the liquid core may also form a hydrogel. In such case the intermediate microcapsule having a hydrogel core is formed.

In the context of this invention it may be desirable to use precursors of microcapsule's shell (including the polyampholyte and/or polyelectrolytes) that exhibit a phase-transition behavior in an aqueous (e.g., water) solution. Some macromolecules may undergo simple phase separation or coacervation (when macromolecules self-assembly) and some may undergo complex coacervation or charge-mediated coacervation (when oppositely charged macromolecules form coacervates) [69]. When using polyelectrolytes to form microcapsule shell it may be desirable to apply complex coacervation, charge-mediated coacervation, precipitation, or combination thereof. Complex coacervation may occur as a result of favorable interactions between different types of macromolecules, such as polyelectrolytes of opposite charge. When using polyampholytes it may be more preferable (but not limited) to apply simple coacervation. Simple coacervation may occur as a result of favorable interactions between identical or highly similar macromolecules (e.g., polyampholytes, polymers or proteins). Precipitation may also occur as a result of favorable interactions between identical or highly similar macromolecules.

In the context of this invention, it may be beneficial to use the polyampholytes that may undergo thermo-responsive phase transition, i.e. a polyampholyte that is a thermo-responsive polymer (as discussed above). In one preferred scenario, the polyampholytes (constituting the microcapsule shell) have an upper critical solution temperature (UCST) that is preferably in the range from 4° C. to 80° C., and more preferable below 50° C. The UCST is defined as the critical temperature above which the microcapsule's shell components in a solution are miscible with the microcapsule's core components. Due to high biocompatibility it may be preferable to use the polyampholytes that not only meet UCST requirements, but also belong to extracellular matrix proteins or their hydrolyzed forms such as gelatin, which shows UCST at 40° C. In another preferred case scenario, the polyampholytes have a lower critical solution temperature (LCST) that is not higher than 42° C., and preferably in the range of 4° C. to 37° C. The LCST is defined as the critical temperature below which the microcapsule's shell components in a solution are miscible with the microcapsule's core components. To fulfill the UCST and/or LCST requirements the polyampholytes may be mixed with other macromolecules having thermo-responsive properties (e.g., N-isopropylacrylamide, ELP, IDP) or macromolecules having gel-stabilizing properties (e.g., chitosan, alginate, hyaluronic acid, polyacrylic acid, polyethylene glycol, etc.).

In view of the above, where the polyampholyte is a thermo-responsive polymer step (b) of the method of producing the microcapsule may comprise producing an intermediate microcapsule by changing the temperature so as to induce physical cross-linking of the thermo-responsive polymer to achieve solidification in the shell phase, i.e. to produce a thermoreversible gel. Thereafter the performance of step (c) covalently links the thermo-responsive polymers together such that the gel is maintained even after the temperature change is reversed.

The temperature may be changed to a temperature from above 0° C. to below 40° C., and may involve raising the temperature (e.g. where the thermoresponsive polymer is one such as Matrigel™) or lowering the temperature (e.g. where the thermoresponsive polymer is one such as gelatin or a gelatin derivative). In preferred embodiments the temperature is below 30° C. and more preferably the temperature is cooled to a temperature that is above 0° C. but below 10° C., most preferably to about 4° C. The temperature may be maintained at this level until the thermoreversible gel is formed e.g. for a period up to an hour, preferably for a period of 1 to 45 minutes, or more preferably for a period of 15 to 30 minutes.

Relevant to this invention, upon liquid-liquid phase separation the polyampholyte may form a liquid coacervate film (liquid shell) entirely enveloping a liquid core comprising a dilute phase of the same polyampholyte. For example, previous reports have proven that single and multi-layered coacervates may form inside water-in-oil droplets [49], core-shell coacervate formation inside the water-in-oil droplets [76]. However, in none of these reports, the generation of capsules and/or encapsulation of cells have been accomplished.

Without being bound to a particular theory the polyhydroxy compound and/or antichaotropic agent added to the solution comprising a polyampholyte, may facilitate the coacervation of the polyampholyte constituting the liquid shell, when both the polyampholyte and antichaotropic agent and/or polyhydroxy compound are mixed together. When two or more polyampholytes are present in the same aqueous mix, upon liquid-liquid phase separation the resulting liquid shell may be enriched in polyampholytes entirely enveloping a liquid core made of the diluted suspension of the same polyampholytes. In other scenarios, when two or more polyampholytes are present in the same aqueous mix, upon liquid-liquid phase separation the first polyampholyte may form a liquid shell entirely enveloping a liquid core comprising the second polyampholyte. In some preferred case scenarios, it may be desirable to form a liquid core comprising a coacervate that has different chemical structure from a coacervate forming a liquid shell. In more complex scenarios, upon liquid-liquid phase separation the liquid shell may envelop multilayered-coacervates, where each layer envelops an inner liquid core, semi-liquid core, or a hydrogel core.

The polyhydroxy compound and/or antichaotropic agent added to the solution comprising a polyampholyte, may also facilitate the precipitation of the polyampholyte constituting the shell, when both the polyampholyte and salt and/or polyhydroxy compound are mixed together. When two or more polyampholytes are present in the same aqueous mix, upon liquid-solid phase separation the resulting solidified shell may be enriched in polyampholytes entirely enveloping a liquid core made of the diluted suspension of the same polyampholytes. In other scenarios, when two or more polyampholytes are present in the same aqueous mix, upon liquid-solid phase separation the first polyampholyte may form a solidified shell entirely enveloping a core comprising the second polyampholyte. In some preferred case scenarios, it may be desirable to form a core comprising a polyelectrolyte that has different chemical structure from a polyelectrolyte forming a shell.

Relevant to this invention, the polyampholyte may be mixed with other macromolecules such as polyhydroxy compounds (e.g., dextran) and allowed to phase separate into one phase enriched in a polyampholyte and another phase enriched in a macromolecule. Upon phase separation the polyampholyte may form a shell entirely enveloping a liquid core enriched in a macromolecule (e.g., polyhydroxy compound). Without being bound to a particular theory the macromolecule constituting the liquid core may facilitate the coacervation and/or precipitation of the polyampholyte constituting the shell, when both the polyampholyte and macromolecule are mixed together. In some scenarios, when two or more polyampholytes are present in the same aqueous mix with a macromolecule, upon liquid-liquid or liquid-solid phase separation the polyampholytes may form a single liquid shell or a single solid shell entirely enveloping a liquid core enriched in a said macromolecule. In other scenarios, when two or more polyampholytes are present in the same aqueous mix with a macromolecule, upon liquid-liquid or liquid-solid phase separation the polyampholytes may form a multi-layered film (multiple shells) entirely enveloping a liquid core enriched in macromolecule, and where the number of layers (shells) surrounding the core corresponds to the number of polyampholytes. For example, two liquid layers surrounding the core may comprise two polyampholytes, three layers surrounding the core comprising three polyampholytes, etc.

In the context of this invention the polyampholyte may be replaced with polyelectrolyte to accomplished liquid-liquid phase separation and form a liquid shell enriched in the said polyelectrolyte.

In some circumstances it may be beneficial to use a mixture of polyampholyte and polyelectrolyte to create an intermediate microcapsule with a solidified shell. For example, the use of a mixture comprising gelatin and chitosan may allow formation of a shell comprising both polymers. The use of a mixture constituting a gelatin and gum arabic may allow formation of a shell comprising both polymers. The use of a mixture constituting a gelatin and alginate may allow formation of a shell comprising both polymers. Experienced in the art and based on literature examples will be able to identify many different composite mixtures suitable for formation of a liquid or solidified shell.

As disclosed in this invention, the aqueous solution containing polyampholyte and the aqueous solution containing polyhydroxy compound may form two aqueous phases with a shared solvent, whereas the solvent may include salts. When encapsulated in water-in-oil droplet, the aqueous solution containing polyampholyte may form an outer (shell) liquid phase and the aqueous solution containing polyhydroxy compound may form an inner (core) liquid phase. The liquid core and/or liquid shell may comprise a single cell or more than two cells. In a preferred scenario the liquid core and not the shell comprise encapsulated cell(s). In another preferred scenario the liquid core and/or shell comprise biological species and/or entities (e.g., nucleic acids, viruses, microorganisms). In one set of embodiments the liquid core and/or liquid shell may comprise solid particles, such as metal nanoparticles, mineral particles, polymer particles, or composite particles. The size of said particle is preferentially from 10 nm to 10 μm.

The aqueous solution containing polyhydroxy compound mixed with polyampholyte may phase separate into two aqueous phases, where polyampholyte may be enriched in one liquid phase and polyhydroxy substance may be enriched in another liquid phase. When liquid-liquid phase separation occurs, the polyampholyte and polyhydroxy compound may be unevenly distributed between the two phases. As revealed in this disclosure, when encapsulated in water-in-oil droplets the polyampholyte preferentially accumulates in an outer (shell) liquid phase and the polyhydroxy compound preferentially accumulate in an inner (core) liquid phase, inside the said water-in-oil droplet. In the context of this invention it may be desirable for polyampholyte to be enriched in the outer liquid phase and the polyhydroxy substance to be enriched in the inner liquid phase. Furthermore, the dynamic viscosity of liquid shell and liquid core is in the range 0.1 to −100 cP (centipose) and preferably in the range of 1.0 to 10 cP. (The dynamic viscosity can be measured at 22° C. using atomic force microscopy or a viscometer.) In a preferred scenario the liquid core contains a cell, or multiple cells. In another preferred scenario the liquid core and/or shell contains any biological species (e.g., nucleic acids, virus particles, microorganisms, etc.).

### Converting the Water-In-Oil Droplets to Microcapsules

Throughout the text of this section the terms of monomer and precursor have been used interchangeably and have the same meaning. Likewise, solidification and gelation have been used interchangeably and have the same meaning.

Without being bound to a particular theory, the liquid transition (transformation) to a gel state, such as liquid shell transformation to a gelled (solidified) shell, may be achieved when the external conditions are changed either during ongoing liquid-liquid phase separation (e.g., liquid shell and liquid core phase separation), or after liquid-liquid phase separation has occurred. For example, the liquid-liquid phase separation and/or the liquid to gel transition, may be facilitated by the inter- and intra-molecular interactions driven by salt-induced dehydration. The dehydration may also lead to dynamic arrest of gel, or gel-like state. Temperature change, irradiation, pH change, ions (monovalent, divalent and multivalent), osmotic pressure difference, chemical concentration gradients, may also lead to dynamic arrest of gel or gel-like state.

In addition, without being bound to a particular theory, under some conditions (e.g., using high charge density polymers, pH far from pI, low salt concentration, temperature, etc.,), core and shell formation may be driven be a precipitation (liquid-solid phase separation). Without being bound to a particular theory, solid shell formation may be achieved when the external conditions are changed such as when the inter- and intra-molecular interactions arise due salt-induced dehydration. Temperature change, irradiation, pH change, ions (monovalent, divalent and multivalent), osmotic pressure difference, chemical concentration gradients, may also lead to precipitation and by extension a solid shell formation.

In the context of this invention, upon core/shell formation (driven either by liquid-liquid phase separation, liquid-solid phase separation or combination of both), the phase constituting the shell may be converted to a gel. When gel forms the intermediate-microcapsule forms. In some cases, the precursors (monomers, pre-polymers, polymers) may form a solidified shell (an intermediate-microcapsule) either contemporaneously (during liquid-liquid phase separation) or sequentially (after liquid-liquid phase separation). In some cases, the formation of the intermediate-microcapsule may occur without a clear liquid shell formation. This may occur when precursors (e.g., polyampholyte) are being continuously deposited onto the outer shell, while polyhydroxy compound simultaneously forms an inner core. This may also occur when precursors (e.g., polyampholyte) precipitate into a solidified (gelled) state, while polyhydroxy compound simultaneously forms an inner (liquid) state.

Without being bound to a particular theory, during the transition from liquid state to the gelled state, the precursors constituting the shell join and form a non-covalently cross-linked gel (solidified shell). In the context of this invention, the solidified shell comprises the precursors (such as a monomeric, pre-polymeric or polymeric species) that may be further cross-linked covalently upon activation by photo-initiator and/or irradiation and/or chemical agent, or any combination thereof. In some cases, the covalent bonds comprise carbon-carbon bonds, disulfide bonds, amide bonds, or ether bonds. The precursors may constitute polyampholytes, polyelectrolytes, or synthetic polymers or any combination thereof. As revealed below in some circumstances it may be beneficial to use the precursors (polyampholyte chains) that are chemically modified (e.g., comprising cross-linkable moieties). The shell precursor may be loaded into water-in-oil droplets during encapsulation (emulsification) step.

Various means may be employed to convert the liquid state to a solidified state (gel) and skilled in the art will be aware of standard methods to induce gelation of the liquid phase. Gelation (solidification) of the liquid shell may be achieved by chemical, enzymatic and/or physical methods. The liquid state may be converted to a solidified state (gel), upon heating, cooling, desalting, pH change, metal complexation, irradiation, precipitation, coacervation, glassy transition, colloidal aggregation, enzymatic or chemical treatment, or any combination thereof. The gelation may be induced by the temperature leading to reversible formation of intermolecular bonds between the individual monomers constituting the intermediate-microcapsule's shell. In the present disclosure the intermediate-microcapsule's shell may form a thermo-reversible gel. In some cases, the intermediate-microcapsule's shell may form a physically cross-linked gel during cooling, or heating, as a result of the inter-molecular forces between the monomers constituting the shell. Physical cross-linking may happen due to chain entanglements of monomers. In some cases, a cationic polyelectrolyte or polyampholyte may interact with an anionic polyelectrolyte or polyampholyte, and lead to a solidified shell. In some scenarios, the intermediate-microcapsule's shell may be ionically cross-linked via electrostatic attraction between two groups of opposite charge. In some cases, the monomers constituting the shell may be ionically cross-linked via metal coordination (e.g., calcium, cobalt, barium) or charged ions (e.g., sulfate). In some cases, the solidification of the liquid shell may be achieved using polyelectrolyte(s), and/or a mixture of polyelectrolyte(s) and polyampholyte(s), where the polyelectrolyte(s) may act as a reversible cross-linker (mold). For example, chitosan may serve as a cross-linker (mold) under neutral pH conditions (pH 7.0), while poly(ethylenimine) may serve as a cross-linker (mold) under alkaline pH conditions (pH 10.5). In some scenarios when liquid shell is comprising a block copolymer the solidified shell may form through glassy junction points. In some cases, the solidified shell may comprise a composite mixture comprising the polyampholyte and macromolecules including some non-limiting example such as synthetic polymers, poly(L-lactic acid), poly(glycolic acid), poly(caprolactone), pol(urethane), glycosaminoglycans, chitosan, hyaluronic acid, poly(acrylic acid), or their modified forms.

In a preferred scenario of the disclosure, the liquid shell is converted to a gel, while core remains in a liquid, or semi-liquid state. In the experiments involving encapsulation of cells or biological species it may be beneficial to use temperature induced gelation to convert the outer liquid phase into the solidified state (hardened shell). In some circumstances the temperature induced gelation of the inner liquid phase comprising traces of polyampholyte may also result in the increased viscoelasticity of the core. In other circumstances both the liquid shell and liquid core may be converted to a gel. When both liquid phases (e.g., constituting the core and shell) are converted to a gel, the gel comprising a shell may have lower water content than the gel comprising the core.

Upon the solidification (and/or precipitation) of a shell, the, e.g. water-in-oil, droplet is termed “intermediate microcapsule” and may be considered as a new type “water-in-oil droplet” that can be named in different terms such “gel-in-oil droplet”, “microcapsule-in-oil”, “water-gel-in-oil droplet”, “bead-in-oil”, “hydrogel-in-oil”, etc. However, critical to the disclosure, upon solidification of a shell (including partial-gelation, or partial-solidification) the resulting intermediate-microcapsule may be released into aqueous environment by destabilizing the water-oil interface (or gel-oil interface) and/or by bursting (breaking) water-in-oil droplet (or gel-in-oil droplet, or capsule-in-oil). Breaking water-in-oil droplets is a well-known and may be achieved chemically, thermally, by dialysis, by extraction, or using an electrical field. Typically, in the context of this disclosure an excess of aqueous buffer is added to the emulsion droplets and microcapsules are released by a process known as deemulsification. Deemulsification may involve chemical reagents, temperature, dialysis, extraction, using an electrical field, etc. During deemulsification process the water-oil (or gel-oil) interface becomes unstable, and inner content of droplets may merge with an aqueous buffer and as a result the encapsulated intermediate-microcapsules may become freed (released) into aqueous buffer. The carrier oil may be removed or replaced in order to enhance or promote deemulsification process. For example, the carrier oil containing surfactant may be replaced with a carrier oil containing no surfactant. Repeating removal of carrier oil process one, two, three or more times the surfactant concentration in the carrier oil may drop below a critical concentration such that water-oil (or gel-oil) interface becomes unstable. As it is reveled here, and as known in the state-of-the-art, the carrier oil containing surfactant (e.g., HFE-7500 with fluorosurfactant) may be mixed (or replaced) with another type of carrier oil (e.g. perfluorooctanol), in order to induce destabilize the water-oil (or gel-oil) interface and release the intermediate-microcapsules. In some circumstances (e.g., when the intermediate-microcapsules are suspended in aqueous buffer) it may be desirable to separate (collect) the intermediate-microcapsules by sedimentation. When traces of the carrier oil are present in the same mix as the intermediate microcapsules it may be desirable to remove or replace the carrier, until a desirable purity is obtained. For example, removal of carrier oil may be repeated two, three or more times until a desirable purity of the intermediate-microcapsules is obtained (e.g., carrier oil contamination below 0.1% (w/v)).

Once dispersed in aqueous solution the intermediate-microcapsule remains intact and does not burst. Moreover, the intermediate-microcapsule not only remains intact but also may retain at least one cell. In other embodiments, once dispersed in aqueous solution the intermediate-microcapsule may retain other biological entities (e.g., nucleic acids, viruses, large macromolecules) and remains intact. The intermediate-microcapsule carrying cells and/or other biological entities can be processed through multistep laboratory procedures such as pipetting, centrifugation, etc., and still retain encapsulated species. The intermediate-microcapsule may be dispersed in different aqueous buffers and still retain encapsulated species. The retention of encapsulated species may depend on the size, molecular weight and charge of the encapsulated species. When a solidified shell comprises polyampholyte, polyelectrolyte, or synthetic polymer that is known to be sensitive to pH change, salts, ions, metal ion chelating agents, temperature or irradiation in those circumstances it may be beneficial to avoid incubation of the intermediate-microcapsule in the conditions that would melt, damage or decompose the solidified shell. It may be desirable to avoid exposing the intermediate-microcapsule to the stimulus or stimuli that cause the intermediate-microcapsule degradation or damage, including for example poor quality, clumping or/and aggregation. For example, when a solidified shell comprises a gelatin it may be beneficial to process the intermediate-microcapsule at temperatures below the gelation temperature (such as gelation temperature of the shell), which is preferably below 40° C., and more preferably below 30° C. and even more preferably below 20° C. and optimally in the range of 0° C. to 8° C.

### Step (c) Covalent Cross-Linking

Step (c) of the method comprises forming intermolecular covalent cross-links with the one or more covalently cross-linkable groups to form a microcapsule comprising a semi-permeable shell of covalently cross-linked polyampholyte and/or polyelectrolyte and a core.

In particular, as described in more detail below, (c) may comprise exposing the shell phase or the thermoreversible gel of the intermediate microcapsule to a chemical agent, an enzyme, irradiation or heat, or any combination thereof, to covalently cross-link the polyampholyte and/or polyelectrolyte.

In one example, (c) comprises exposing the shell phase or the thermoreversible gel of the intermediate microcapsule to an enzyme, such as an aminotransferase.

In a further example, (c) comprises activating the chemically cross-linkable groups by exposing the shell phase or the thermoreversible gel of the intermediate microcapsule to an initiator such as chemical-initiator (e.g., tetramethylethylenediamine, ammonium persulfate), a photo-initiator (e.g., lithium phenyl-2,4,6-trimethylbenzoylphosphinate), a thermal initiator (e.g., heat), a radiative-initiator (e.g., visible or UV light), or any combination thereof.

When the solidified shell of intermediate-microcapsule is covalently cross-linked the final microcapsule of this invention is formed, hereinafter for simplicity referred as microcapsule. The covalent crosslinking should be understood as the process during which two or more molecules (e.g. precursors) are chemically joined by a covalent bond. The shell of the intermediate-microcapsule may comprise the precursors that form covalent bonds upon a reaction with a chemical agent, upon irradiation, or upon enzymatic reaction. In the context of this invention, the shell of intermediate-microcapsule may preferentially comprise the precursors that form covalent bonds upon a reaction with a photo-initiator. Independently of the cross-linking reaction used, upon the polymerization process, the solidified (gelled) shell may form a chemically (covalently) cross-linked gel. Person experienced in the art will be aware of different cross-linking agents and strategies that may covalently cross-link individual monomers into a polymer mesh. Likewise, person experienced in the art will be aware of different cross-linking moieties that can be incorporated into monomers. In some cases, the monomers (e.g., polyampholyte) may be composed of a variety of chemical groups such as amino acid side chains that can be chemically modified in order to introduce the desirable cross-linking moieties.

In order to covalently cross-link the solidified shell of the intermediate-microcapsules and obtain the final-microcapsule (or just microcapsule) individual precursors (monomers, oligomers, pre-polymers) have to begin to polymerize and form a covalently cross-linked and elastic shell. For this purpose, an initiator and/or an accelerator, electromagnetic radiation (irradiation), temperature, pH changes, and any combinations thereof may be applied. In the context of this invention, an initiator and/or accelerator may be added in the same suspension where the intermediate microcapsule is present. A large variety of accelerators and initiators are available and will be known to those experienced in the art. In the context of this invention, an accelerator may be a chemical reagent or an agent which initiates or facilitates the polymerization reaction (process). An accelerator is expected to accelerate polymerization reaction rate. In some cases, an accelerator may speed up the activation of an initiator (e.g., via the generation of free radicals) used to then activate monomers and, thus, initiate a polymerization reaction. An initiator may be a reagent, an agent or species capable of initiating a polymerization reaction by activating one or more chemical moieties (e.g., acrydate, methacrylate,—SH group) in the polymerization reaction. In some circumstances of this disclosure it may be preferential to use fast activation of an initiator in order to accomplish fast polymerization. The activation of precursors may occur via the generation of free radicals or activation of chemical groups. In the context of this invention the use of an initiator such as lithium phenyl-2,4,6-trimethylbenzoylphosphinate (LAP) may be applied in the cross-linking reaction (polymerization reaction).

In some cases, speed up of polymerization process may be achieved by other means such as heat, irradiation (e.g., visible light, UV light, etc.), pH change, etc. In the context of this invention it may be advantageous to explore the irradiation as a mean to increase polymerization (cross-linking) rate, or in some other cases, a use of heat. When certain precursors constituting the microcapsule's shell contain light-sensitive properties the polymerization may be initiated by exposing microcapsule to a visible light, UV light. Irradiation may involve a combination of visible light combined with a sensitizer, or UV light combined with a sensitizer, or combinations thereof. An example of a sensitizer may be riboflavin, 3-hydroxypyridine, etc.

During the polymerization reaction a covalently cross-linked and elastic shell may form. In some scenarios, an accelerator may speed-up polymerization by activating a polymerization initiator. The polymerization reaction can be conducted in the presence of a single or many accelerators. Likewise, the polymerization reaction may be conducted in the presence of a single or many initiators. It may be necessary to optimize the polymerization conditions such as concentration of accelerator and/or initiator and/or intensity of light, to obtain microcapsule with desirable properties and cross-linked shell. In a set of embodiments of this disclosure an initiator may be applied to cross-link individual precursors (monomers, oligomers, and/or pre-polymers) into a covalently cross-linked and elastic shell. An initiator and accelerator may be water-soluble, oil-soluble, or may be both water-soluble and oil-soluble. For example, an accelerator TEMED and an initiator APS are commonly used in polymerization reaction may be suitable for this disclosure. Other type of initiators, azo-based initiators, may be used as thermal based initiators that may generate free radicals thermally. In a preferred scenario of this disclosure an accelerator or initiator is added to the same suspension in which an intermediate microcapsule is suspended, in order to initiate a cross-linking (polymerization) reaction. However, an accelerator and/or initiator may be also added to the carrier oil containing a surfactant prior to water-in-oil droplet generation, or after collection of water-in-oil droplets. An accelerator and/or initiator may be also added to the carrier oil containing a surfactant, after water-in-oil droplet collection off-chip, or during water-in-oil droplet generation process. An accelerator and/or initiator may be also added to the carrier oil with or without a surfactant, and then such oil mixed with water-in-oil droplets collected off-chip. An accelerator and/or initiator may be also added to the aqueous phase prior to water-in-oil droplet generation. When cross-linking reaction is performed within the emulsion droplets the resulting microcapsules may be released into aqueous environment by bursting (breaking) water-in-oil droplet following the same procedure as described above for the intermediate-microcapsule.

Cross-linking (polymerization) reaction may vary depending on multiple factors such as the size of the microcapsule, type of an accelerator or initiator, irradiation, temperature, when and whether an accelerator or initiator is added, when and how electromagnetic radiation is applied, the precursor concentration, etc. In the context of this invention the cross-linking (polymerization) may be completed in about 0.3, 0.5, 0.6, 0.7, 0.8, 1.0, 2.0, 3.0, 4.0, 5.0, 6.0, 7.0, 8.0, 10, 11, 12, 13, 14, 15, 20, 30, 60, 120 minutes. In some cases, the cross-linking may be complete after more than about 0.3, 0.5, 0.6, 0.7, 0.8, 1.0, 2.0, 3.0, 4.0, 5.0, 6.0, 7.0, 8.0, 10, 11, 12, 13, 14, 15, 20, 30, 60, 120 minutes, or even longer.

In the context of this disclosure it may be highly desirable that upon the cross-linking of the solidified shell, the encapsulated cell viability is minimally affected by the polymerization (cross-linking) reaction. It may be beneficial to replace or dilute the aqueous solution in which microcapsules are suspended one or several times with a buffer or cell culture medium, in order to remove activator and/or initiator present in the aqueous solution with microcapsules. For example, it is possible to envision that upon a cross-linking reaction some chemical species toxic to the cells may be present or appear in the same solution (e.g. unreacted accelerator, activated initiator, new radical species, etc.). Removing those chemical species by replacing or diluting the aqueous solution in which microcapsules are present may reduce encapsulated cell cytotoxicity and/or damage of encapsulated species.

As one of the relevant examples but not limiting example of this disclosure, it details the use of the polyampholyte namely gelatin derivative. The rheological properties of the gelatin-based hydrogels can be controlled by the degree of substitution, polymer concentration, initiator concentration, irradiation conditions, etc. [77]. Other proteins and oligopeptides, including but not limited to collagen, laminin, elastin, fibrin, silk fibroin, proteoglycans, glycosaminoglycans, or their hydrolyzed forms may be modified with reactive chemical moieties as detailed below (and that are known for experienced in the art) and applied for generating the microcapsules. As disclosed in this invention the microcapsule may be generated by cross-linking the modified gelatin monomers into an elastic covalently linked polymer mesh, where the gelatin monomers are chosen from one of the following gelatin derivatives: gelatin methacryloyl, gelatin methacrylamide, gelatin methacrylate or gelatin acrylamide. However, gelatin and more broadly almost any polyampholyte may be modified with groups (moieties) that participate in the cross-linking reaction. The number of possible cross-linking moieties, groups, substitutions and compounds is enormous, and person experienced in the art will be able to identify various cross-linking moieties suitable for generating the microcapsules. The non-limiting examples of cross-linking moieties that may benefit the disclosed invention comprise but are not limited to acrylates and its derivatives such as methacrylate, methacrylic anhydride, 2-(Dimethylamino)ethyl methacrylate, 2-(Diethylamino)ethyl methacrylate, 2-Carboxyethyl acrylate, 3-Sulfopropyl methacrylate, 2-Sulfoethyl methacrylate, ethylene glycol di(meth)acrylate, ethylene glycol diacrylate, di(ethylene glycol) diacrylate, tetra(ethylene glycol) diacrylate, ethylene glycol dimethacrylate, di(ethylene glycol) dimethacrylate, tri(ethylene glycol) dimethacrylate, glycidil methacrylate, methacrylic acid, acrylic acid, methacrylic anhydride, etc. Some cross-linking moieties that may benefit the disclosed invention comprise but are not limited to acryloyl, methacryloyl and their derivatives such [2-(Methacryloyloxy)ethyl]trimethylammonium chloride, 2-(Acryloyloxy ethyl)trimethyl ammonium chloride, [3-(Methacryloylamino)propyl]trimethylammonium chloride, acryloyl pyrrolidine, acryloyl piperidine, etc. Some cross-linking moieties that may benefit the disclosed invention comprise but are not limited to are derivatives of acrylamide such as methacrylamide, N-[3-(Dimethylamino)propyl]acrylamide, N,N-(1,2-Dihydroxyethylene) bisacrylamide, N,N-Methylenebisacrylamide, N-isopropylacrylamide, etc. Some other cross-linking moieties that may benefit the disclosed invention may comprise but are not limited to vinylsulfone, vinylpyrrolidone, thiol, azide, alkynes, carboxylated poly-L-lysine, hydroxyproprionic acid, hydroxy phenol, diisocyanates, poly(epoxy) polymer, polyacrylic acid, and other.

Other shell cross-linking strategies may include modifying the precursor with phenolic hydroxyl group, which can then be cross-linked enzymatically as exemplified in reference [78]. In another aspect the precursor may be functionalized with norborene. As was shown previously, norborene functionalized gelatin can be crosslinked with poly(ethylene glycol) dithiol using thiolene photo-click reaction [79]. In another aspect the monomers can be cross-linked using disulfide bonds thereby forming a cross-linked shell. Overall, experienced person in the field will be able to identify the modified polyampholytes, polyelectrolytes or polymers that can be successful applied to produce a microcapsule with a cross-linked shell following the concept/method/procedure disclosed here.

Noteworthy, changing the cross-linker moiety density, different size and type of crosslinker, and/or monomer amount in the shell may allow tuning the mechanical properties (e.g., elasticity, porosity) of microcapsules.

In the context of this invention it is beneficial to cross-link intermediate-microcapsule's shell by supplying the (bio)chemical reagent (e.g., cross-linking agent, photo-initiator, catalyst) externally, through the solution in which the intermediate-microcapsules are dispersed. Covalent cross-linking of the intermediate-microcapsule may be initiated upon irradiation. For example, the shell of the intermediate-microcapsules may be converted to a covalently cross-linked polymer mesh (shell) by supplying the photo-initiator externally and activating the said photo-initiator with a light. Photo-initiator may be used to initiate the polymerization and cross-linking of monomers (precursors) into a 3D polymer mesh. Different photo-initiators are known to the experienced in the art. Photo-initiators suitable for this disclosure may comprise but not limited to Norish Type I and Norish Type II initiators, Amine synergists. Some non-limiting examples of photo-initiators include: 2,2-Dimethoxy-1,2-diphenylethan-1-one, 2-Hydroxy-2-methyl-1-phenylpropanone; 1-Hydroxy-cyclohexylphenylketone; Benzophenone; Isopropyl thioxanthone; 2-ethylhexyl-(4-N,N-dimethyl amino)benzoate; Ethyl-4-(dimethylamino)benzoate, Water-soluble TPO based nanoparticle photoinitiator; Phenylbis(2,4,6-trimethylbenzoyl)phosphine oxide; 2,4-Diethyl-9H-thioxanthen-9-one; Benzoin; Benzophenone; Benzoin methyl ether; Bis(4-tert-butylphenyl)iodonium p-toluenesulfonate; Bis(4-tert-butylphenyl)iodonium perfluoro-1-butanesulfonate; Bis(4-tert-butylphenyl)iodonium triflate; Tris(4-tert-butylphenyl)sulfoniumperfluoro-1-butanesulfonate; Triphenylsulfonium perfluoro-1-butanesufonate; 1-Naphthyl diphenylsulfonium triflate; Triarylsulfonium hexafluoroantimonate salts; 2-Hydroxy-2-methylpropiophenone; 1-Hydroxycyclohexyl phenyl ketone; 2-Benzyl-2-(dimethylamino)-4′-morpholinobutyrophenone; Lithium phenyl-2,4,6-trimethylbenzoylphosphinate; Diphenyl(2,4,6-trimethylbenzoyl)phosphine oxide; Lauryl acrylate; Michler's ketone and their derivatives thereof. In the context of this invention it may be advantageous to use lithium phenyl-2,4,6-trimethylbenzoylphosphinate (LAP) as a photo-initiator.

The precursors (polyampholytes, polyelectrolytes or polymers) lacking chemical moieties (substitutions) may be successful applied to produce a microcapsule when mixed with another macromolecule (e.g., modified polyampholytes, polyelectrolytes or polymers) having groups (moieties, substitutions) required for a cross-linking reaction to occur. For example, collagen, gelatin or other polyelectrolytes may be mixed with polyacrylic acid, acrylamide or other molecules that serve as cross-linkers themselves, or have chemically active groups (e.g. cross-linking groups), necessary for a covalent cross-linking of the intermediate-microcapsule. In some scenarios the precursors lacking chemical moieties (substitutions) may be cross-linked into polymer shell by activating them with chemical agents that produce reactive groups. The precursors could be modified before loading them in water-in-oil droplets, or once the intermediate-microcapsule is formed. The non-limiting examples of chemical agents that produce reactive groups facilitating the cross-linking reaction between individual monomers comprising the shell include EDC (1-ethyl-3-(˜3-dimethylaminopropyl) carbodiimide hydrochloride) or homologs thereof for generation of carboxyl-to-amine reactive crosslinking groups. N-Hydroxysuccinimide esters, imidoester, pentafluorophenyl ester, hydroxymethyl phosphine or homologs thereof may be applied to produce amino reactive groups. Maleimide, haloacetyl (bromo- or iodo-), pyridyldisulfide, thiosulfonate, vinylsulfone or homologs thereof may be used to generate sulfhydryl-reactive groups. Hydrazide or alkoxyamine or homologs thereof may be used to generate aldehyde-reactive groups (e.g., oxidized sugars (carbonyls)). Diazirine or aryl azide or homologs thereof may be used to generate photoreactive groups. Isocyanate or homologs thereof to generate hydroxyl (nonaqueous)-reactive groups. Dithiobis(succinimidylpropionate) or homologs thereof to generate amine-reactive groups. Succinimidyl 4-(N-maleimidomethyl)cyclohexane-1-carboxylate (SMCC) or Sulfo-SMCC or homologs thereof to generate amine-to-sulfhydryl crosslinking. These non-limiting examples illustrate that strategies for crosslinking the solidified shell are vast and person experienced in the art will be able to identify a suitable cross-linking strategy for generating a microcapsule.

In one specific example disclosed here the solidified shell of the intermediate-microcapsule is covalently cross-linked during radical polymerization reaction (e.g. applying TEMED and APS). Other non-limiting examples of radical initiators include chemical agent called Irgacure 2959. The shell of the intermediate-microcapsule might be also covalently cross-linked by using chemical agents that act as cross-linkers the non-limiting examples include aldehydes (e.g., glutaraldehyde, glyceraldehyde), N,N′diallyltartardiamide (DATD), cystamine, N,N′-Bis(acryloyl)cystamine (BAC), dimethyl suberimidate, sodium tetraborate (borax), and others. The solidified shell of the intermediate-microcapsule may be covalently cross-linked using natural cross-linking agents for proteins (e.g., genipin).

In some circumstances, and as revealed below, the shell precursors (polyampholytes, polyelectrolytes or polymers) lacking synthetic cross-linking moieties (substitutions) may be successful applied to produce a microcapsule by using a suitable enzymatic reaction (e.g., transaminase). For example, the shell might be cross-linked by using aminotransferases such as transglutaminase to link lysine to glutamine residues [80]. In some other cases, the solidified shell might be cross-linked using amine oxidase enzyme (e.g. lysyl oxidase) that converts lysine moieties into highly reactive aldehydes.

In some cases, the cross-linking agent or photo-initiator may be soluble in the liquid shell, and/or in the liquid core. The cross-linking agent or photo-initiator may be soluble in the carrier oil with or without a surfactant. The cross-linking agent or photo-initiator may be soluble in the aqueous buffer in which solidified intermediate-microcapsules are dispersed. The cross-linking agent or photo-initiator may not be present within the water-in-oil droplets during their formation. The cross-linking agent or photo-initiator may be supplied externally after formation of the intermediate-microcapsule. Therefore, the experienced person in the field will notice that non-modified polyampholytes, polyelectrolytes or polymers may be successful applied to produce final-microcapsules when the intermediate-microcapsule is treated with a cross-linking agent supplied externally, and where the cross-linking agent crosslinks individual monomers via covalent carbon-carbon, disulfide, carbon-oxygen, carbon-nitrogen, or other covalent bonds.

The final-microcapsule may contain labile bonds where non-limiting examples include an ester bond (e.g., cleavable with an acid, base, or hydroxylamine), a Diels-Alder linkage (e.g., cleavable by heating), a vicinal diol bond (e.g., cleavable with sodium periodate), a sulfone linkage (e.g., cleavable via a base), a silyl ether bond (e.g., cleavable with an acid), a phosphodiester bond (e.g., cleavable with hydrolase (e.g., endonuclease), a glycosidic linkage (e.g., cleavable with amylase) or a peptide linkage (e.g., cleavable with a protease) amongst others.

An intermediate microcapsule may comprise about 1, 10, 100, 1′000, 10′000, 100′000, 1′000′000, 10′000′000, 100′000′000, 1000′000′000, or more chemical moieties (e.g. methacrylate, methacrylamide) participating in the cross-linking reaction for generating a final microcapsule. In other examples, an intermediate microcapsule may comprise at least 1, 10, 100, 1′000, 10′000, 100′000, 1′000′000, 10′000′000, 100′000′000, 1000′000′000, or more chemical moieties participating in the cross-linking reaction for generating a final microcapsule.

In the context of this invention it may be preferable when upon the covalent cross-linking the shell may form porous polymer with pore diameters ranging from 0.1 to 200 nm, and preferably in the range of 10-100 nm. Moreover, as reported in the disclosure, upon the covalent cross-linking the microcapsule's shell become thermo-resistant (thermo-stable) and does not decompose at elevated temperatures (e.g., at approximately 95° C.).

The final microcapsules may have pores about 0.1 nm, 0.5 nm, 1 nm, 2 nm, 3 nm, 5 nm, 7 nm, 10 nm, 15 nm, 20 nm, 30 nm, 40 nm, 50 nm, 60 nm, 70 nm, 80 nm, 90 nm, 100 nm, 150 nm or 200 nm. In some cases, a microcapsule may have pores at least about 0.1 nm, 0.5 nm, 1 nm, 2 nm, 3 nm, 5 nm, 7 nm, 10 nm, 15 nm, 20 nm, 30 nm, 40 nm, 50 nm, 60 nm, 70 nm, 80 nm, 90 nm, 100 nm, 150 nm or 200 nm. In some cases, the pores may vary in size and be in range of about 0.1-1 nm, 0.1-10 nm, 1-10 nm, 0.1-100 nm, 1-100 nm, 10-100 μm, 0.1-200 nm, 1-200 nm, 10-200 nm. Pore size may be determined by scanning electron microscopy or by determining the diffusion of compounds of different molecular weight.

A further aspect of the disclosure reveals the microcapsule where the covalently cross-linked shell comprises proteinaceous biomaterial. The term “proteinaceous” refers to a biomaterial containing, resembling, or being made from a protein(s), peptides, oligopeptides or polypeptides, or any combination thereof. It should be understood that proteinaceous shell may comprise composite mix and/or include additives such as sugars, synthetic polymers (e.g., PEG) in order to change the properties of the shell (e.g., porosity, stiffness, elasticity, mechanical stability, etc.). The protein(s), peptides, oligopeptides or polypeptides constituting the shell do not need to be the major precursor or ingredient of the microcapsule's shell to ensure the applicability of the microcapsule. In another aspect the outer shell of microcapsule may be composed of the proteoglycans containing heparin, chondroitin-sulfate, dermatan-sulfate, heparan-sulfate, hyaluronan, hyaluronic acid, or derivatives thereof. In one aspect the disclosure reveals the outer shell of a microcapsule that is composed of natural biopolymer or a fragment comprising such polymer. Natural biopolymers are found in nature and are preferentially are found in mammals, animals, plants or microorganisms. Nature biopolymer may comprise proteins and/or polysaccharides and/or nuclei acids, or fragments thereof.

However, the outer shell of microcapsule may comprise synthetic polymers or a fragment comprising synthetic polymer. Synthetic polymer is preferably analogous to natural biopolymer. In one aspect, the outer shell of microcapsule may comprise or contain the proteinaceous material such Matrigel™ or Geltrex™, or synthetic analogs as reviewed elsewhere [81].

In the context of this invention it may be desirable to obtain a microcapsule where the outer shell comprises a polyampholyte belonging to the group of the extra-cellular matrix oligopeptides, peptides or proteins such as collagen, mucin, laminin, elastin, fibrin, proteoglycans, glycosaminoglycans, or their hydrolyzed forms (e.g. gelatin) thereof, or any combination thereof. In one aspect of the disclosure the outer shell of microcapsule preferentially comprises the proteins and/or polypeptides and/or oligopeptides and/or peptides that belong to, or are derived from the collagen, laminin, gelatin, elastin, fibrin, silk fibroin, fibronectin, vimentin, poly-L-lysine.

In one example, the microcapsules may be produced by:
i) Injection of the first aqueous fluid comprising the polyampholyte in a water-in-oil droplet generation device;
ii) Injection of the second and/or other aqueous fluid(s) comprising polyhydroxy substance(s) and/or cells in a water-in-oil droplet generation device;
iii) Forming the water-in-oil droplets comprising the polyampholyte, the polyhydroxy substance, and the cells;
iv) Providing sufficient time for the outer shell and inner core to form inside the water-in-oil droplets whereas the outer shell may be enriched in the polyampholyte, and the inner core may be enriched in the polyhydroxy substance; and where the cells are preferentially distributed in the core;
v) Temperature-, salt- or pH-induced, or combination thereof, gelation of the shell; vi) Formation of the intermediate microcapsule with a solidified shell;
vii)Breaking the emulsion droplets to release the intermediate microcapsules having a solidified shell;
viii) Further stabilization of the intermediate microcapsule by covalently cross-linking the shell;

### Functionalizing the Microcapsules

The microcapsules may have one or more types of functionality at their inner and/or outer surface. The microcapsules may have one or more types of functionality at their core. In particular, in the context of this invention it may be advantageous to functionalize the microcapsules with components providing the desired chemical or biological properties, such as hydrophilicity, hydrophobicity or altered cell adhesion properties. In some preferred scenarios the cross-linked shell of microcapsule may contain the peptide those amino acid sequence comprises Arg-Gly-Asx, Gly-Arg-Gly-Asx-Tyr, Gly-Arg-Gly-Asx-Ser, Tyr-Ile-Gly-Ser-Arg, Gly-Tyr-Ile-Gly-Ser-Arg-Gly, Ile-Lys-Val-Ala-Val, Lys-Arg-Glx, Arg-Glx-Asx-Val, Gly-Arg-Glx-Asx-Val-Tyr, Leu-Gly-Thr-Ile-Pro-Gly, Pro-Asx-Ser-Gly-Arg, Arg-Asx-Ile-Ala-Glu-Ile-Ile-Lys-Asx-Ala, Asp-Gly-Glx-Ala, Val-Thr-X-Gly, Val-Gly-Val-Ala-Pro-Gly or X-B-B-X-B-X, or any combination thereof. Where X is a hydrophobic amino acid, and B is positively charged basic amino acid, namely arginine or lysine.

The microcapsules revealed in this disclosure may also contain biomolecules and/or components such as oligonucleotides (e.g., DNA or RNA primers, nucleic acid fragments) that may become incorporated into the microcapsule during cross-linking (polymerization) reaction. The incorporation of biomolecules (e.g., oligonucleotides, peptides) may be achieved via covalent or non-covalent association with the microcapsule shell or the microcapsule core, or combination thereof. In some cases, the oligonucleotides and/or peptides may be supplemented in the aqueous phase during (dispersed phase) during water-in-oil droplet formation. In some other cases, the oligonucleotides and/or peptides may be supplemented in the aqueous phase in which the intermediate microcapsule is suspended. In a preferred scenario the DNA oligonucleotides are incorporated via acrydite moiety that becomes cross-linked to the microcapsule during the polymerization reaction. In other scenarios it may be preferential to have the DNA oligonucleotides incorporated into microcapsule via disulfide bond. For example, the oligonucleotides may be attached to the acrydite moiety by a disulfide linkage resulting in a composition comprising a microcapsule-acrydite-S-S-oligonucleotide linkage. Other biomolecules and/or components may be incorporated following the same principle. The incorporation of biomolecules and/or components such as oligonucleotides may be achieved either during microcapsule formation, when the intermediate-microcapsule is formed, or when final-microcapsule is formed, following formation, or any combination thereof. The plurality of oligonucleotides attached to the microcapsule may have identical sequence, or different sequences. For some application it may be desirable to use the plurality of oligonucleotides that may include functional sequences (e.g., a fragment of gene specific sequence, sequencing adapter, PCR adapter, etc.). It should be understood, that the intermediate-microcapsule and/or final-microcapsule may be attached to one or more different types of multi-functional oligonucleotides, or that the intermediate-microcapsule and/or final-microcapsule may be attached to a variety of species that are multi-functional.

In some scenarios it may be advantageous to use components (e.g., the oligonucleotides, peptides, lipids) that are covalently attached to the plurality of biomolecules (polyampholytes, polyelectrolyte, polymers, monomers). In this context the said biomolecules serve as composite monomers (precursors) of microcapsule shell and upon polymerization reaction form a chemically cross-linked shell with said components (e.g. DNA primers) attached to the monomers. When using composite monomers (and/or polymers), the oligonucleotides may be incorporated into the microcapsule's shell and/or core during the formation of the intermediate-microcapsule, or it may be incorporated into the microcapsule's shell and/or core after the intermediate-microcapsule is produced. Likewise, when using composite monomers (and/or polymers), the oligonucleotides may be incorporated into the microcapsule's shell and/or core during the formation of the final-microcapsule, or it may be incorporated into the microcapsule's shell and/or core after the formation of the final-microcapsule.

Some non-limiting examples of biomolecules and species that may also be attached, entangled or coupled to microcapsules include DNA (e.g., chromosomes, polynucleotides, oligonucleotides, nucleotides, etc.), RNA (mRNA, rRNA, tRNA, riboswitch, viral RNA, ribonucleotides, oligoribonucleotides, etc.), peptide polynucleotides, organic molecules, proteins, polypeptides, carbohydrates, saccharides, sugars, lipids, enzymes, optical barcodes, DNA barcodes, antibodies or antibody fragments, fluorophores, detergents, a locked nucleic acid (LNA), nucleic acid analogue, inhibitors (protease inhibitors, nuclease inhibitors), chelating agents, reducing agents, oxidizing agents, assay probes, chromophores, dyes, surfactants, polymers, pharmaceuticals, radioactive molecules, preservatives, antibiotics, aptamers, and combinations thereof. It should be understood that biomolecules and/or species may be coupled (attached) to microcapsules by any suitable method known to person experienced in the art, including covalent and non-covalent linkages (e.g., C—C bonds, C—N bonds, C—O—C bonds, ionic bonds, van der Waals interactions, hydrophobic interactions, ionic interactions, encapsulation, entanglement, etc.).

In some scenarios it may be advantageous to attach biomolecules and/or components that are capable of capturing a particular type of sample component, including components that may comprise nucleic acid and/or comprise nucleic acids from lysed cells. In some other scenarios it may be advantageous to attach biomolecules and/or components that are capable of capturing a particular type of sample component, including components that may comprise biomolecules secreted, released or produced by the cells irrespectively if cell lysis is involved or not. For example, a microcapsule may comprise a ligand (capture probe) capable of binding (capturing) a cell or biomolecule. It may be advantageous to use the capture probe comprising an antibody, antibody fragment, receptor, protein, oligopeptide, peptide, amino acids, enzyme cofactors, vitamins, small biochemical molecules or any other species capable of interacting with biomolecules on the surface of the cells, or intracellular biomolecules of the cells. In some scenarios it may be preferable for capture probe to interact with extracellular or intracellular species (e.g., proteins, lipids, sugars, nucleic acids) via inter-molecular interactions so that a particular cell type can be captured by the microcapsule, or that a particular biomolecule of the cell may be captured by the microcapsule.

### Breaking the Microcapsule to Release Encapsulated Species

In a further aspect the present invention provides a method of releasing the inner content of a microcapsule described herein, wherein the method comprising breaking the semi-permeable shell of the microcapsule. In particular, the released inner content may comprise the at least one biological entity (e.g. if the at least one biological entity has not been subjected to lysis prior to breaking the semi-permeable shell) and/or products produced by or from the biological entity while inside the microcapsule.

In broad terms the microcapsule of this invention may be degraded, disrupted, broken, or dissolved upon exposure to one or more stimuli in order to release the inner content, and optionally the encapsulated biological entity, into the external environment/surroundings of the microcapsule. Those experienced in the art may be able to identify suitable strategies for breaking the microcapsule taking into an account the teaching herein and the material used to generate the microcapsule. In some cases, the final microcapsule may be broken upon exposure to particular chemical species, pH change, exposure to light, exposure to enzymes, etc. In one set of specific embodiments the microcapsule is broken (dissolved) upon enzyme-driven hydrolysis reaction. The material comprised in the microcapsules (e.g., polyampholyte and polyhydroxy compound) may be solubilized when exposed to a particular stimulus or stimuli (e.g., chemical species, enzyme). The microcapsule may be degraded or dissolved at elevated temperature or it may stay intact at elevated temperature. Depending on a particular application it may be desirable to have a microcapsule that is thermostable, yet biodegradable upon exposure to a chemical or biological reagent (e.g., hydrolase enzyme). As disclosed in this invention the microcapsule may be a thermostable microcapsule that is biodegradable upon enzymatic (protease) treatment. Herein, the thermostable means that microcapsule does not disintegrate when incubated at elevated temperatures for extended period of time (e.g., at least 10 min at 95° C.).

When a microcapsule is formed from a polyampholyte comprising peptide bonds, i.e. one belonging to a group of proteinaceous materials, the degradation of microcapsule shell may be achieved enzymatically, upon contact with protease enzyme. When a microcapsule is formed from a polyampholyte comprising degradable chemical crosslinkers, such as cystamine or its analogs, the degradation of the microcapsule shell may be achieved upon contact with a chemical degrading agent that may induce reduction, oxidation or chemical modification. For example, reducing agents such as dithiothreitol (DTT), β-mercaptoethanol, tris(2-carboxyethyl) phosphine (TCEP), or (2S)-2-amino-1,4-dimercaptobutane (DTBA), or combinations thereof may break (degrade, cleave) the disulfide bonds formed between monomers forming the cross-linked shell.

The length of time required for microcapsule degradation (dissolution) will depend on application and may vary from 0.1 second to 24 hours or longer. The microcapsule may degrade instantaneously upon exposure to the appropriate stimuli, or it may degrade over time. As disclosed in this invention the microcapsule may be degraded over time upon exposure to protease enzyme. For example, a microcapsule may be degraded (dissolved) upon exposure to protease enzyme between 0.1-60 minutes, or longer. Increasing the concentration of protease may result in faster dissolution of a microcapsule. In a preferred scenario the dissolution of a microcapsule occurs at time window shorter than 60 min, and more preferably within 20 minutes.

As disclosed in this invention in a preferred scenario the covalent bonds of cross-linked shell are broken (e.g., using protease), the microcapsule loses integrity and is broken (dissolved). A microcapsule may be broken in suspension, however, in some scenarios it may be preferable to break a microcapsule inside another partition, such as a water-in-oil droplet, or a well, such that the microcapsule degrades within the said partition and encapsulated biological entity/entities are released within the said partition upon the appropriate stimulus (e.g., presence of protease). Within the said partition (e.g., the aqueous droplet), the degraded microcapsule may release encapsulated biological entity/entities (e.g., cells or nucleic acids encodes by the cell) inside the partition.

After the microcapsule is broken the released entities may still be associated with the poly hydroxy composition of the core. Accordingly, in some embodiments the method of releasing further comprises a step of hydrolyzing the polyhydroxy compound of the released inner content by contacting with a hydrolase enzyme, so as to release the at least one biological entity from (association with) the polyhydroxy compound. As above, the hydrolase enzyme for hydrolysis of the core may be a glycosidase, a dextranase, or an amylase. One skilled in the art can select a suitable hydrolase enzyme based on the identity of the polyhydroxy compound. The ability of the component of the core to be hydrolyzed in this manner can advantageously be used to improve the recovery of the biological entity/entities from the microcapsule, particularly where the biological entity/entities is nucleic acid.

Types of Samples Suitable for Use with Microcapsules

The microcapsule of this disclosure may be used with any suitable biological entity or non-biological species and/or samples. Biological entities may be derived from human and non-human sources. In some cases, biological entities may be derived from mammals, non-human mammals (e.g. monkeys), rodents (e.g. mice, rat), rabbits, camels, pigs, cows, horses, goats, sheep, dogs, cats, amphibians, reptiles, hens, birds, fish, insects, slugs, microbes, algae, fungi, archaea, bacteria, parasites, unicellular micro-organisms, etc. Biological entities may comprise a variety of cells including but not limited to eukaryotic cells, prokaryotic cells, fungi cells, archaea, bacteria, unicellular microorganisms, cells from multi-cellular microorganisms, human cells, reproductive cells, stem cells, induced pluripotent stem cells, cancer cells, patient cells, etc. Biological entities may be derived from a variety of cells but not limited to human cells, eukaryotic cells, prokaryotic cells, fungi cells, archaea, bacteria, unicellular microorganisms, cells from multi-cellular microorganisms, reproductive cells, stem cells, induced pluripotent stem cells, cancer cells, patient cells, etc. Biological entities may comprise but not limited to the content of cells such as the contents of a single-cell, or the contents of multiple cells. Biological entities may comprise a cell-free biomolecules, such as circulating nucleic acids (e.g., DNA, RNA), viruses, exogenic nucleic acid molecules, DNA or RNA fragments, etc.

Biological entities and non-biological samples may comprise live or dead cells, whole or damages cells, DNA, RNA, a particular type of nucleic acid (e.g., complementary DNA (cDNA), double stranded DNA (dsDNA), single stranded DNA (ssDNA), plasmid DNA, cosmid DNA, chromosomal DNA, genomic DNA (gDNA), viral DNA, bacterial DNA, mtDNA (mitochondrial DNA), messenger RNA, ribosome RNA, transport RNA, microRNA, dsRNA, ribozyme, riboswitch, viral RNA and other types of RNA or DNA molecules), fragments of nucleic acids, DNA barcodes (e.g., barcode sequences, nucleic acid barcode sequences, barcode molecules), organelles, ribosomes, mitochondria, cell nucleus, aptamers, viruses, nucleotides, deoxynucleotide triphosphate (dNTPs), dideoxynucleotide triphosphates (ddNTPs), peptide polynucleotides, organic molecules, proteins, polypeptides, carbohydrates, polysaccharides, oligosaccharides, saccharides, sugars, lipids, enzymes, optical barcodes, antibodies or antibody fragments, fluorophores, detergents, a locked nucleic acid (LNA), nucleic acid analogue, inhibitors (protease inhibitors, nuclease inhibitors), chelating agents, reducing agents, oxidizing agents, assay probes, chromophores, dyes, surfactants, polymers, pharmaceuticals, radioactive molecules, preservatives, antibiotics, cell culture medium, growth factors, serum, vitamins, peptides, amino acids, acidic or basic solutions, temperature-sensitive compounds, pH-sensitive species, light-sensitive species, magnetic or polymer particles, metals, metal ions, salts, aqueous buffers, cofactors, activators, lipids, oils, detergents, and combinations thereof. It should be understood that this is not a complete list and the samples will vary depending on a particular application or assay.

It should be understood that a sample and/or a biological entity may be obtained from different sources or from the environment. For example, a sample or biological entity may be obtained from the organisms, tissues, biopsies, bodily fluids, aspirates, air, agricultural samples, soil samples, petroleum samples, water samples, dust or space samples. In some cases, biological samples may be man-made products.

### Kits for Producing a Microcapsule

In another aspect, the present invention provides a kit for making the microcapsule described herein, the kit comprising:
(a) a polyhydroxy compound and/or an antichaotropic agent;
(b) a polyampholyte comprising one or more covalently cross-linkable groups; and optionally (c) a microfluidic chip.

In another aspect, this disclosure provides a kit for making the microcapsule described herein, the kit comprising:
(a) a polyampholyte and/or polyeletrolyte (suitable for constituting the microcapsule's shell); and
(b) a polyhydroxy compound and/or antichaotropic agent (suitable for constituting the microcapsule's core); and optionally
(c) a microfluidic chip or device.
   Where the kit comprises a microfluidic chip, this may comprise a plurality of microchannels configured to form a droplet from a first solution comprising the polyhydroxy compound and/or the antichaotropic agent, a second solution comprising the polyampholyte, and optionally a fluid comprising a carrier oil, further optionally with a surfactant.

The polyampholyte and/or polyelectrolyte may be as described herein. Similarly, the polyhydroxy compound and/or the antichaotropic agent may be as described herein. In one example the polyhydroxy compound and/or the antichaotropic agent are provided in a first solution and the polyampholyte and/or polyelectrolyte are provided in a second solution.

The kit may further optionally comprise instructions for making the microcapsule and/or encapsulating the biological entity (e.g., cells). The protocol (instructions) may also provide guidelines of utilizing the microfluidic device for producing the water-in-oil droplets. The kit may further comprise the carrier oil, optionally supplemented with a surfactant that is suitable to stabilize the water-in-oil droplets that are produced. As mentioned above any suitable sample (biological or non-biological origin) may be incorporated into the fluidic droplets.

The kit may include additional reagents, for example, the kit may include buffer for washing (rinsing) the microcapsules, and/or a photo-initiator. The kit may include additional consumables, for example, microfluidics consumables such as tubing, syringes, needles, etc. The kit may include additional devices, for example, light emitting device for photo-illumination and initiation of polymerization process.

In some cases, the kit may include cell culture ingredients, buffers, vitamins, supplements necessary for cell culture and growth in 3D environment. In some other cases, the kit may include RNA or DNA amplifying enzymes (e.g. RT and PCR enzymes), nucleoside triphosphates or their analogues, primers, buffers, etc. and instructions for using microcapsules for amplifying nucleic acids. In some other cases, the kit may include components necessary for improving and/or maintaining encapsulated cell viability.

### Applications

### The Use of Microcapsules for Cell-Based Assays and for Other Type of Assays

For the sake of simplicity and clarity the terms “culture” also covers such terms as incubation, in vivo culture, in vitro culture, harvesting, maintaining, propagation, replication, expansion, growth, division. It should be understood that in the context of this disclosure the terms “in vitro culture” and “in vivo culture” signifies any biological process that may occur during prolonged periods (>12h) of culture. For example, cells may divide, grow, expand, migrate, attach, interact with other cells, interact with biomolecules or substrate, cell may secrete biomolecules, absorb molecules, produce biomolecules, release biomolecules, etc.

It should be noted that culture methods described herein are described as in vitro. While in vitro methods are preferred it is noted that the methods can also be performed in vivo. Such in vivo methods are non-therapeutic unless they are identified as being therapeutic methods.

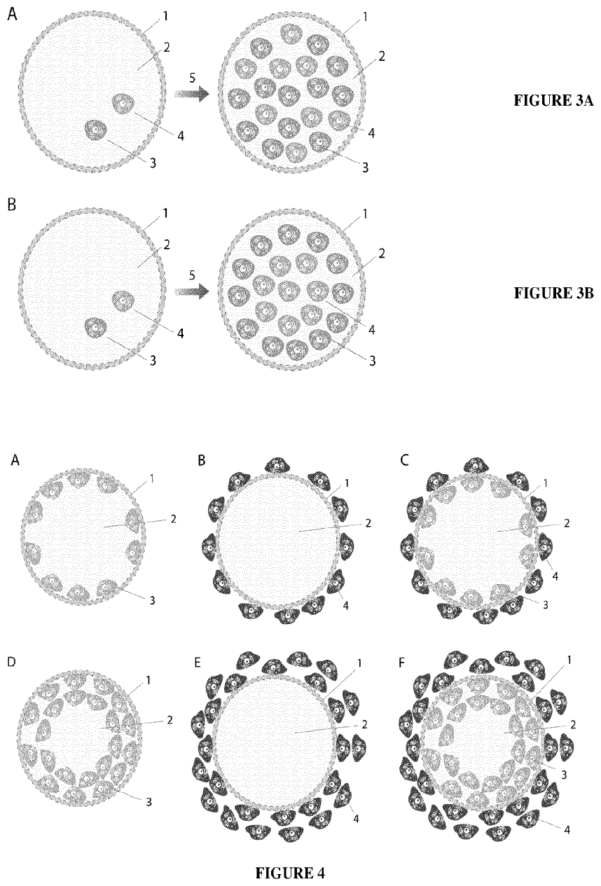

The microcapsule of this disclosure may be used as a biocompatible compartment for encapsulating a cell, or more than one cell, whereas the said cell(s) may be cultured and allowed to form 3D cell assemblies (structures) (FIG. 2). The microcapsule may provide a 3D microenvironment and enable in vitro or in vivo culture of 3D cell culture. The encapsulated cells may form 3D cell structures (assemblies) such as spheroids, organoids, tumoroids, tissues, assemblies, clumps and other cell clusters. The microcapsule carrying one, two, three, four, five or more than five cells can be cultured in suitable in vitro or in vivo conditions to enable formation of spheroids, organoids, tumoroids, tissues and other types of 3D cell structures (FIG. 3). At any time point the encapsulated cell(s) may be analyzed using a large variety of techniques some of the non-limiting examples of which include bright field and fluorescence microscopy, flow cytometry, FACS, immune-assays, antibody-based assays, standard molecular, genetic engineering, biochemistry and/or cell biology techniques. The physiological and/or biological functions and/or features of the encapsulated cells (e.g., growth, shape, division, metabolic activity, etc.) may be evaluated using a variety of biological, chemical and physical techniques.

In some scenarios of this disclosure it may be preferable to culture the individual cells for extended periods of time to allow cell division and appearance of the daughter cells. In other scenarios it may be advantageous to expand the encapsulated cells into a clonal population of cells, and/or masses of cells, including but not limited to spheroids, organoids, tumoroids, tissues and other types of 3D cell assemblies (3D cell structures). In other scenarios it may be advantageous to encapsulate multiple cells and culture the encapsulated cells for extended periods of time and generate diverse populations of cells, and/or masses of cells, including but not limited to spheroids, organoids, tumoroids, tissues and other types of 3D cell structures.

The encapsulated cells may preferentially occupy (distribute, reside) at the inner core of the microcapsules. The microcapsule's shell may serve as a support (substrate) for cells to attach to (FIG. 4). For example, the microcapsule's shell may serve as a substrate for adhesion (attachment) of encapsulated cells (FIG. 4A). It may also serve as a substrate for adhesion for the cells present outside the microcapsule (FIG. 4B). The encapsulated cell(s) may attach to the inner surface of the shell and form monolayers, multilayers or other complex cell structures (FIG. 4D). Likewise, the cell(s) residing outside the microcapsule may attach to the outer surface of the shell and form monolayers, multilayers or other complex cell structures (FIG. 4E). In some scenarios, the cells inside the microcapsule and cells outside the microcapsule may attach to the microcapsule's shell and form monolayers (FIG. 4C) or multilayers/complex structures (FIG. 4F).

In some scenarios of this disclosure a microcapsule may provide a biocompatible compartment for culture (co-culture) of two or more cell types, each in different or the same microcapsule (FIGS. 3 and 5). For example, co-encapsulated cells can communicate with each through secreted factors, or physically by interacting with each other via cell-cell interactions. This type of biochemical communication can be bidirectional (FIG. 5A) and/or unidirectional (FIG. 5B). In another example, the plurality of microcapsules carrying cells of one type may be suspended in a solution (suspension) having a different type(s) of cells and allowing encapsulated cells to biochemically communicate with cells present outside the microcapsules (e.g., cells in a suspension); whereas the encapsulated cells and the cells in a suspension are maintained physically separated from one another by the microcapsule's shell (FIGS. 5C and 5D). In another scenario, the plurality of microcapsules carrying one type of cells can be mixed (suspended) with plurality other type(s) of microcapsules carrying encapsulated cell(s) of different type(s); and allowing both cell types to communicate biochemically (e.g., via soluble factors), yet at the same time remain physically separated from each other (FIGS. 5E and 5F). In these and other scenarios the biochemical communication by cells can be bidirectional (FIGS. 5A, C and E) or unidirectional (FIGS. 5B, D and F)

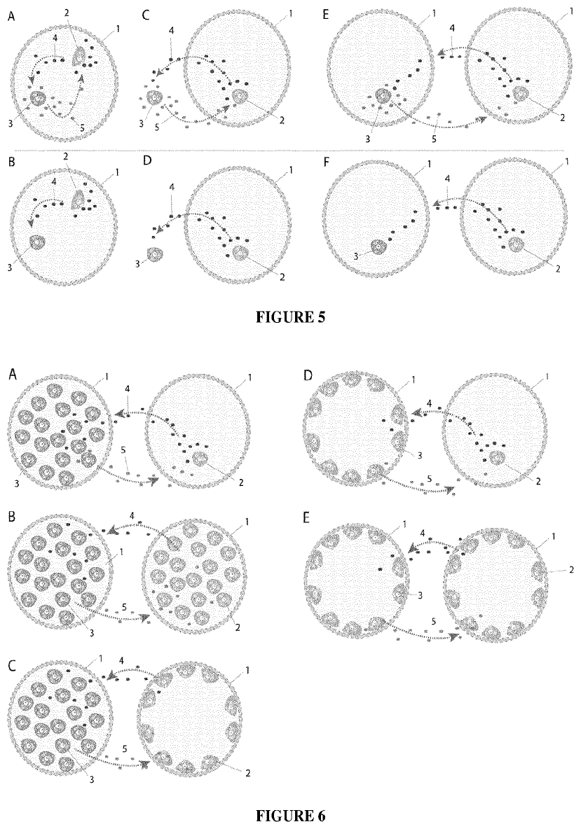

In some other scenarios of this disclosure, the plurality of microcapsules comprising 3D cell assemblies can be incubated with plurality of microcapsules carrying encapsulated cell(s) of different type and allowing cells in different microcapsules to communicate biochemically (e.g., via soluble factors), yet at the same time remain physically separated from each other (FIG. 6). In non-limiting examples, microcapsules carrying a 3D cell assembly may be incubated with other capsules carrying one cell (FIGS. 6A and 6D), or more than on cell. In some other non-limiting examples, microcapsules carrying a 3D cell assembly may be incubated with microcapsule also carrying a 3D cell assembly (FIGS. 6B, 6C and 6E). Encapsulated cells in one microcapsule may interact and/or biologically respond (e.g., by altering gene expression, reorganizing the cytoskeleton, etc.) to soluble factors secreted by the cell(s) present in another microcapsule.

In some other scenarios of this disclosure, the microcapsules carry one or more cells that may attach to the microcapsule's shell and allowed to interact with cells outside the microcapsule, where the cells outside the microcapsule may attach to the surface of the same microcapsule (FIG. 7). The cells in suspension (e.g. present outside the microcapsule) in some cases may attach to the outer surface of the microcapsule having encapsulated cell(s) and remain physically separated by the shell for a long period of time (>12 h). For example, the microcapsule's shell may serve as a substrate for adhesion (attachment) of cell that reside outside the microcapsule, so that two cells (one cell outside microcapsule and another cell inside the microcapsule) may communicate with each other via soluble factors (FIG. 7A). In another non-limiting example, the microcapsule may also serve as a substrate for adhesion for the cells that reside inside the microcapsule, so that cells inside and outside the microcapsule may communicate via soluble factors without touching each other physically (FIG. 7B). The encapsulated cells or cells outside the microcapsule may form layers of cells and communicate via soluble factors without touching each other physically (FIGS. 7C and 7D). In yet another scenario, 3D cell assemblies that are present inside and outside the microcapsule may interact with each other through soluble (secreted) biological factors (FIGS. 7E and 7F).

In some non-limiting examples, a microcapsule may be used as compartment for performing a cell-based assay (e.g., screening assay) that is generally known as cell cytotoxicity assay [34, 35]. For example, the two-cell binding assay (see for example ref [37]) where the T-cell and target cell interacts via T-cell receptor may be conducted inside the microcapsule. As yet another example, a microcapsule may serve as a compartment for performing two-cell binding assay where the dendritic and target cell interacts via cell receptor(s) and/or secreted factors (biomolecules). As yet another example, a microcapsule may serve as compartment for performing two-cell interaction assay (see for example ref [38]) where the natural killer (NK-cell) and target cells are interacting biochemically and/or physically, within the same microcapsule. Overall, any type of cell, two cells, three cells, or many cells may be loaded into a microcapsule and cells allowed to interact physically (e.g., cell-cell interaction) or biochemically (e.g. soluble factors, secreted molecules, etc.) and at any step in the procedure the cells of interest may be enriched and/or retrieved from the microcapsule for further analysis.

In some scenarios, cell-based assay conducted inside the microcapsules may involve any type of antibody binding assay. For example, antibody binding assay may involve one type of cells (e.g., the cancer cells) and second type of cells (e.g., the immune cells) co-encapsulated in microcapsules, and then allowed to interact via soluble factors and/or interact physically by establishing cell-cell contact, within the same microcapsule. The antibody binding events against cancer cells may record as exemplified previously droplet microfluidics format (see for example ref [36]). Upon binding the cancer cells, the antibodies produced by the immune cell may be detected using a variety of techniques (e.g., sandwich ELISA assay, fluorescence-based assay) that will be known to the expert in the art, and the immune cells of interest may be enriched and/or retrieved from the microcapsule for further analysis.

In some other scenarios, screening assay of secreted proteins may rely on the formation of tertiary complex that is too large to pass the microcapsule shell. For example, the secreted proteins (e.g., antibodies) may bind soluble biomolecule (e.g. antigen) and form tertiary complex that is too large in size to pass the microcapsule shell. Therefore, the microcapsules that have a cell of interest may be identified by recording the tertiary complex formation. The semi-permeability of microcapsule shell ensures that immunoassay reagents, including the antigen, can diffuse and distribute evenly among the microcapsules, yet only those microcapsules that contain cells producing functional protein (e.g., antibody) will result in the formation of tertiary complex (e.g., antibody-antigen complex). Following tertiary complex formation, washing the unbound antigens and/or antibodies and/or immunoassay reagents may enhance the detection efficiency of antibody-antigen binding events. The antibody producing cells of interest may be enriched and/or retrieved from the microcapsule for further analysis.

In some scenarios, screening assay of secreted proteins may rely on the formation of tertiary complex on a solid support. For example, the secreted proteins may bind ligand(s) attached to micro- or nano-particles. These types of assays have been previously reported using water-in-oil droplet format [82, 83]. When said particles are co-encapsulated with the cells of interest, the cells may produce biomolecules (e.g. antibodies, cytokines, proteins) that bind ligand(s) attached to micro- or nano-particles. The binding events may be recorded using fluorescence-assay, ELISA-assay or any type of immune-assay that will be known to the expert in the art. The cells of interest may be enriched and/or retrieved from the microcapsule for further analysis.

The plurality of microcapsules carrying the plurality cell(s) of interest may be generated by encapsulating the plurality of cells into plurality of liquid droplets and converting the plurality of liquid droplets into plurality of microcapsules. It should be understood that the liquid droplets may be water-in-oil droplets, water-in-water droplets, or it could be water-in-air droplets. In a preferred scenario the liquid droplets are water-in-oil droplets. The process of converting the liquid droplet to microcapsule is described above, and exemplified in several examples below. Encapsulation of cells and/or entities and/or reagents, media etc., may be performed using a microfluidics device, capillary assembly or droplet generation device.

The microcapsules dispersed in aqueous environment may be subjected to one, or more analytical or experimental treatments (e.g. microcapsules can be washed and dispersed in growth medium, or mixed with immunoassay reagents, etc.) in order to perform biological assay on encapsulated cells, or culture encapsulated cells. The semi-permeability of the microcapsule's shell may ensure that encapsulated cell(s) receive the nutrients when the microcapsule is suspended in cell growth media. In the context of this invention it may be desirable to have microcapsule's shell that is permeable to low molecular weight molecules and compounds and prevents larger biomolecules from entering, or leaving, the microcapsule. For example, it may be advantageous of using microcapsules that allow bidirectional diffusion (e.g., in and out of the microcapsules) of molecules, nutrients and compounds having molecular weight smaller than approximately 300 kDa and even more preferably smaller than 200 kDa; and whereas the same microcapsule (microcapsule's shell) prevents larger biomolecules from entering, or leaving, the microcapsule where the nutrients, molecules and compounds have molecular weight larger than approximately 200 kDa. As mentioned above, by changing the porosity of the shell it may be possible to tune the permeability of the microcapsules.

When using the microcapsules disclosed here the phenotype and/or genotype of the encapsulated cell(s) may be evaluated using a larger variety of laboratory techniques, methods, protocols and assays that will be known to a skilled person in the art. Whereas the phenotype may be described as a trace (feature) that can be measured or evaluated experimentally by observation, fluorescence readout, microscopy readout, absorbance readout, enzymatic, regulatory or binding activity readout, metabolic activity readout, or other any other readout, and whereas the genotype is a trace (feature) that may be measured or evaluated by any technique or method involving nucleic acid analysis (e.g., sequencing, PCR, RT-PCR, fluorescence measurement, hybridization, absorbance, fluorescence in situ hybridization, etc.). In principle, any assay or analytical method that is used in analytical chemistry, biochemistry, cell biology, molecular biology, genetic engineering, synthetic biology, biotechnology, biomedicine field may be applicable to the disclosed microcapsules. Likewise, any assay or analytical method that is used to analyze biological features or genetic make-up of the cells may be applicable onto encapsulated cells and/or biological species.

In some circumstances the plurality of microcapsules carrying the plurality of cell(s) and/or other biological entities may be subjected to a variety of laboratory techniques available in a research laboratory such as fluorescence and bright field microscopy, flow cytometer, FACS, dialysis, incubation at desirable temperature or buffer, etc. Non-liming examples of biological assays compatible with microcapsules include antibody binding assay, ELISA, cell viability assay, metabolic function assays, protein synthesis and analysis assay, nucleic acid assays, lipid assays, carbohydrate assays, fluorescence staining and enzyme-based fluorescence assays, and any other cell-based and molecular assays that are commonly used to determine the phenotype or genotype information of the encapsulated cells. The microcapsules carrying encapsulated single-cell or population of cells may be subjected to multi-step analytical procedures and multi-step biological assays and yet still retain the encapsulated cell(s).

In some circumstances the plurality of microcapsules carrying the plurality of cell(s) and/or plurality of nucleic acids may be subjected to an enzymatic assay(s) the non-limiting examples of which include reverse transcription (RT), DNA and/or RNA replication, DNA and/or RNA amplification, DNA and/or RNA hybridization, DNA and/or RNA fragmentation, DNA and/or RNA modification, DNA and/or RNA ligation, DNA and/or RNA DNA and/or RNA extension, DNA and/or RNA hydrolysis, DNA and/or RNA synthesis, DNA and/or RNA capture, DNA and/or RNA protection, DNA and/or RNA binding, DNA and/or RNA taggmentation, DNA and/or RNA barcoding, DNA and/or RNA indexing, DNA and/or RNA labelling, DNA and/or RNA conjugation, DNA and/or RNA degradation, DNA and/or RNA assembly, DNA and/or RNA sequencing, whole genome amplification, polymerase chain reaction (PCR), qPCR, RT-PCR, and any other method to obtain a genetic make-up information and/or genome-encoded and/or epigenome-encoded information about the encapsulated cells.

In some circumstances the plurality of microcapsules may comprise a plurality of biological entities or a collection of biological entities (e.g., collection of nucleic acids, proteins, biomolecules) that can be analyzed and/or amplified and/or barcoded, for example, but not limited to using standard molecular and cell biology techniques (e.g., hybridization, RT, PCR, RT-PCR, sequencing etc.). The biological entities may be analyzed and evaluated using fluorescence-based or absorbance-based methods, flow cytometry, FACS, PCR, qPCR, RT-PCR, microscopy, etc.

In another aspect, the invention provides a method of storing cells comprising suspending the microcapsule or a plurality of microcapsules which comprise at least one cell in a storage medium comprising a cryoprotectant and freezing the cells. In some examples, the microcapsules carrying encapsulated cells can be added to a solution having cryoprotectant (e.g., methanol, acetate, dimethylsulfoxyde (DMSO), glycerol, trehalose, glycol, etc.,) and stored in liquid nitrogen, optionally for an extended period of time, such as 6 months or 1 year.

In particular examples of this method of storing, the cell comprised in the microcapsule may be an adherent cell, or an adherent cell culture, which is positioned in the core and is attached to a surface of the shell, particularly an inner surface. Without wishing to be bound by theory, it is thought that the method of storing of the present invention is particularly advantageous for adherent cells as they can be stored adhered to the shell rather than as a suspension.

The microcapsule can be applied for screening the chemical and/or biological compounds that affect cell viability and/or trigger biological response, whereas said method comprises:
i) Culture of encapsulated cell(s) in the presence and in the absence of a chemical and/or biological compound,
ii) Recording the phenotype of interest of cells in the presence and/or in the absence of a chemical and/or biological compound,
iii) Identification of the chemical and/or biological compound that triggered the phenotype of interest.

In the context of this invention the “chemical compound” can be any chemical substance, yet having molecular weight not higher than 100 kDa and more preferably in the range of 0.1-10 kDa. In the context of this invention the “biological compound” can be any biological substance, biomolecule or biochemical compound, yet having molecular weight not higher than 300 kDa and more preferably in the range of 0.1-200 kDa. In the context of this invention the “phenotype of interest” means a change of biological feature or characteristics of the encapsulated cell (e.g., cell death, gene expression, binding or catalytic activity). In the context of this invention the “Identification” may reflect non-limited examples of spectrophotometric approach, microscopy, flow cytometry, FACS, nucleic acid sequencing, fluorescence readout and other approaches or any molecular biology approach known to skilled in the art. Because hundreds, thousands and even millions of the encapsulated cells may be cultured in parallel such approach may provide significant analytical advantages over competing technologies such as 96-well plates. Moreover, since encapsulated cells are cultured in 3D niche (3D environment), upon treatment with the chemical and/or biological compounds it may be possible to identify phenotypes and/or biological features and/or biological characteristics that remain elusive when using standard screening techniques.

In some aspects the invention reveals a method for screening biochemical compounds comprising:
i) The culture of cells inside the microcapsule in the presence or in the absence of a biochemical compound of interest;
ii) Detection of cell response (e.g. cell viability, gene or protein expression, etc,) to the said biochemical compound and comparing to the cell response in the absence to the biochemical compound;
iii) Identifying the biochemical compounds of interest that triggers a cell response

The microcapsule can be applied for screening for a chemical and/or biological compound that affects cell viability and/or triggers a biological response, wherein said method comprises:
i) The culture of encapsulated cells in the presence and in the absence of a selected compound,
ii) Recording the phenotype of interest of cells in the presence and/or in the absence of the selected compound,
iii) Identification of the chemical and/or biological compound that triggered the phenotype of interest.

The microcapsule may be applied for performing cell functional assay in the presence and/or in the absence of a screening compound (the chemical and/or biological compound), wherein said method comprises:
i) Producing the microcapsule comprising two or more cells;
ii) The incubation of two or more cells within the same microcapsule (in the presence and/or in the absence of a screening compound);
iii) Allowing the encapsulated cells to interact physically and/or biochemically;
iv) Recording the phenotype of interest of cells (in the presence and/or in the absence of a selected compound); where in a preferred scenario one of the cells is immune cell; and whereas the other cell(s) may be any cell and preferably a tumor cell;
v) Isolating (enriching, sorting, picking etc.) the microcapsules that contain cells of interest;
vi) Releasing the cells of interest, or lysing the cells of interest;
vii) Optional: identification of the chemical and/or biological compound that triggered the phenotype of interest.

The microcapsule may be applied for performing binding assay in the presence and/or in the absence of a screening compound (the chemical and/or biological compound), wherein said method comprises:
i) Producing the microcapsule comprising two or more cells;
ii) The incubation of two or more cells within the same microcapsule (in the presence and/or in the absence of a screening compound);
iii) Allowing the encapsulated cells to interact biochemically and/or physically; where in a preferred scenario one of the cells is immune cell producing a biomolecule (e.g., antibody) binding to other cell(s); and where the other cell(s) may be any cell and preferably a tumor cell;
iv) Recording the phenotype of interest of cells (in the presence and/or in the absence of a selected compound);
v) Isolating (enriching, sorting, picking etc.) the microcapsules that contain cells of interest;
vi) Releasing the cells of interest, or lysing the cells of interest;
vii) Optional: identification of the chemical and/or biological compound that triggered the phenotype of interest.

The microcapsule may be applied for performing cell functional and/or binding assay in the presence and/or in the absence of a screening compound (the chemical and/or biological compound), wherein said method comprises:
i) Producing the microcapsule comprising a first type of cells, or multiple types of cells; wherein the cells may be labelled before or after isolation in microcapsules, and wherein labeling may involve fluorescence probes, fluorescent protein expression, or any other labeling method.
ii) Producing the microcapsule comprising a second type of cell, or multiple types of cells;
iii) Incubation of the microcapsule comprising a first type of cell or multiple types of cells with the microcapsule comprising a second type of cell or multiple types of cells, in the same aqueous solution; and in presence and/or in the absence of a screening compound;
iv) Recording the phenotype of interest of cells (in the presence and/or in the absence of a selected compound); for example, measuring the fluorescence of cell(s) in the first microcapsule or growth of cells and/or biological features of cells;
v) Isolating (enriching, sorting, picking etc.) the microcapsules that contain cells of interest;
vi) Releasing the cells of interest, or lysing the cells of interest;
vii) Optional: identification of the chemical and/or biological compound that triggered the phenotype of interest.

The microcapsule may be applied for performing cell functional and/or binding assay in the presence and/or in the absence of a screening compound (the chemical and/or biological compound), wherein said method comprises:
i) Producing the microcapsule comprising first type of cells, or multiple types of cells;
ii) Adding the microcapsule comprising a first type of cell or multiple types of cells to the aqueous solution having a second type of cell or multiple types of cells (e.g., in presence and/or in the absence of a screening compound);
iii) Recording the phenotype of interest of cells (in the presence and/or in the absence of a selected compound), for example, measuring the fluorescence of cell(s), growth of cells, biological features of cells, recording the migration of a second type of cells, etc.; and whereas the cells of interests might be the cells in suspension, and/or the cells inside the microcapsule;
iv) Isolating (enriching, sorting, picking etc.) the cells of interest;
v) Optional: Identification of the chemical and/or biological compound that triggered the phenotype of interest.

The microcapsule may be applied for performing cell functional and/or binding assay in the presence and/or in the absence of a screening compound (the chemical and/or biological compound), wherein said method comprises:
i) Producing the microcapsule comprising a first type of cell, or multiple types of cells;
ii) Adding the microcapsule comprising a first type of cell, or multiple types of cells to the aqueous solution having a second type of cell or multiple types of cells (e.g., in presence and/or in the absence of a screening compound);
iii) Incubating the cells for a period of time sufficient for producing biomolecules (e.g. antibodies, cytokines, etc.) that may bind or enter the cells (in the presence and/or in the absence of a selected compound);
iv) Recording the phenotype of interest of cells, for example, after performing ELISA assay, immunoassay, or any other assay, or by recording the biological features of cell(s); and whereas the cells of interests might be the cells in suspension, and/or the cells inside the microcapsule;
v) Isolating (enriching, sorting, picking etc.) the cells of interest;
vi) Optional: Identification of the chemical and/or biological compound that triggered the phenotype of interest.

The microcapsule may be applied for performing protein binding assay in the presence and/or in the absence of a screening compound (the chemical and/or biological compound), wherein said method comprises:
i) Producing the plurality of microcapsules comprising plurality of cells;
ii) Incubating the cells for a period of time sufficient for producing a protein (e.g., antibody) that binds a target (e.g., antigen) of interest (in the presence and/or in the absence of a selected compound);
iii) Recording the protein binding event (e.g., tertiary complex formation) using an immunoassay and preferably using a fluorescence-based readout;
iv) Isolating (enriching, sorting, picking etc.) the cells of interest;

The microcapsule may be applied for performing protein binding assay in the presence and/or in the absence of screening compound (the chemical and/or biological compound), wherein said method comprises:
i) Producing the plurality of microcapsules comprising plurality of cells and plurality of micro- or nano-particles; wherein the said particle carry a ligand for capturing (binding) the protein of interest produced by the cell;
ii) Incubating the cells for a period of time sufficient for producing protein that binds the ligand on particle (in the presence and/or in the absence of a selected compound);
iii) Recording the protein binding event using an immunoassay and preferably fluorescence-assay;
iv) Isolating (enriching, sorting, picking etc.) the cells of interest;
v) Optional: Identification of the chemical and/or biological compound that triggered the phenotype of interest.

In aforementioned examples the encapsulated cell(s) may be released to bulk by treating the capsules with enzyme (e.g., protease, peptidase).

### The Use of Microcapsules in Medicine

As described in the art, microcapsules can be used in medicine, and in particular to deliver therapies to the human or animal body [84-86].

Similarly, the microcapsules described herein may also be used to deliver a therapy to a subject in need thereof. Accordingly, the present invention provides a method of delivering at least one biological entity to a subject for treatment of a disease, a disorder or an injury in the subject, the method comprising administering a microcapsule as described herein to the subject, wherein the microcapsule comprises the at least one biological entity, optionally wherein the at least one biological entity is comprised in the core of the microcapsule.

Similarly, the microcapsule as described herein may be for use in delivering a medical therapy, wherein the microcapsule comprises at least one biological entity for the medical therapy, optionally wherein the at least one biological entity is comprised in the core of the microcapsule.

Also provided in the use of a microcapsule as described herein for the manufacture of a medicament for delivering a treatment to a subject, wherein the microcapsule comprises at least one biological entity for the treatment.

In particular, preferred embodiments the treatment is cell therapy and the at least one biological entity is at least one cell. In one example, the microcapsule encapsulates a plurality of cells that can be implanted at a site of injury in the subject, where the plurality of cells will be released from the microcapsule after implantation (as the microcapsule will disintegrate inside the subject's body). In another example, the microcapsule encapsulates a plurality of cells secreting one or more cytokines (e.g. one or more proinflammatory cytokines) that can be implanted in the subject's body to initiate a therapeutic response by e.g. the immune cells of the subject.

### Further Aspects/Embodiments of the Invention

### Some Aspects on Composition of the Microcapsule's Outer Shell

In one aspect the microcapsule is composed of an elastic, covalently cross-linked shell comprising a polyampholyte; the inner core comprising a polyhydroxy compound belonging to the class of carbohydrates, oligosaccharides, polysaccharides or sugars.

In one embodiment the said microcapsule comprises a cell, or more than one cell.

In one set of embodiments the microcapsule may comprise macromolecules (such as biopolymers, polyelectrolytes or polyampholytes) that undergo liquid-liquid phase separation.

In one set of embodiments the microcapsule comprises the macromolecules that may self-assembly into coacervates and form polyampholyte-rich liquid phase, and a polyampholyte-dilute liquid phase.

In one set of embodiments, the disclosure reveals a microcapsule where the covalently cross-linked shell comprises polyampholyte, which belongs to proteinaceous materials. Herein, the term “proteinaceous” refers to a biomaterial containing, resembling, or being made from a protein(s), peptides, oligopeptides or polypeptides, or any combination thereof.

In one embodiment of the aspects described herein, the outer shell of microcapsule may comprise proteinaceous material such Matrigel™ or Geltrex™, or synthetic analogs.

In one embodiment of the aspects described herein the disclosure reveals the outer shell of a microcapsule that is composed of natural biopolymer or a fragment comprising such polymer. Natural biopolymers are found in nature and are preferentially are found in mammals, animals, plants or microorganisms. Natural biopolymer may comprise proteins and/or polysaccharides and/or nuclei acids, or fragments thereof.

In one set of embodiments the microcapsule comprises the macromolecules that belong to but are not limited to the group of proteins, polypeptides or oligopeptides comprising intrinsically disordered regions, elastin-like polypeptides, synthetic and/or natural biopolymers.

In a specific set of embodiments, the microcapsule's shell comprises the macromolecules having highly repetitive and low complexity amino acid sequences.

In another specific set of embodiments, the microcapsule's shell comprises the proteinaceous biomaterial rich in disorder-promoting amino acids such as glycine and/or proline. and may also contain glutamate, serine, lysine, alanine, arginine and/or glutamine. In a preferred scenario the disorder-promoting amino acids will constitute at least 10% of all amino acids comprising the proteinaceous biomaterial (e.g. polypeptide).

In one specific embodiment, the microcapsule's shell comprises the polypeptide composed of the disorder-promoting amino acids constituting over 30% of polypeptide mass.

It should be understood that proteinaceous material constituting the microcapsule's shell may comprise composite mixture and/or include synthetic polymers (e.g., PEG, poly-L-lysine) that may change the properties of the microcapsule shell (e.g., porosity, stiffness, elasticity, mechanical stability, etc.). Proteinaceous material does not need to be the major ingredient or exclusive precursor of the cross-linked shell.

In one set of embodiments the outer shell of microcapsule is composed of polyampholyte belonging to the group of the extra-cellular matrix polypeptides, oligopeptides, peptides or proteins such as collagen, laminin, mucin, elastin, fibrin, proteoglycans, glycosaminoglycans, or their hydrolyzed forms (e.g. gelatin) thereof, or any combination thereof.

In one set of embodiments the outer shell of microcapsule may comprise synthetic polymers or a fragment comprising synthetic polymer, where the synthetic polymer is analogous or shows 80% similarity to natural biopolymer.

In one particular set of embodiments the outer shell of microcapsule preferentially comprises the proteins and/or polypeptides and/or oligopeptides and/or peptides that belong to, or are derived from the collagen, mucin, laminin, gelatin, elastin, fibrin, silk fibroin, fibronectin, vimentin.

In one set of embodiments, the disclosure provides a microcapsule where the outer shell of a microcapsule may comprise the elastin-like polypeptides, for example, elastin, gluten, gliadin, abductin, resilin, tropoelastin, fibronectin, silks proteins, etc.

In one set of embodiments of the aspects described herein the outer shell of a microcapsule is composed of natural biopolymers or fragment(s) comprising such polymer. Natural biopolymers are found in nature and are preferentially are found in mammals, animals, plants or micro-organisms. Nature biopolymer may comprise proteins and/or polysaccharides and/or nuclei acids, or fragments thereof.

In one specific embodiment the microcapsule shell is composed of gelatin, or gelatin derivatives.

In a preferred embodiment, the polyampholyte has an upper critical solution temperature (UCST) that is in the range from 4° C. to 60° C.

In another preferred embodiment, the polyampholyte has a lower critical solution temperature (LCST) that is in the range from 4° C. to 37° C.

In another specific embodiment, upon a cross-linking reaction the microcapsule becomes thermostable and does not disintegrate during PCR, thermocycling or prolonged incubations at elevated temperatures (e.g. 50-98° C.).

In one set of embodiments, the mass fraction (w/w, weight/weight or w/v, weight/volume) of a macromolecule constituting the shell is chosen in the range of 0.1-50% and more preferably in the range of at 1-10%.

In one set of embodiments, the shell of a microcapsule may contain solid particles, such as metal nanoparticles, mineral particles, polymer particles, or composite particles. The size of said particle is preferentially from 10 nm to 10 μm.

In one set of embodiments, the shell of a microcapsule is from 0.2 to 100 μm thick, and more preferably in the range of 1-10 μm thick.

In one set of embodiments, the pore diameter of a shell may be in the range from 0.1 to 200 nm, and preferably in the range of 1-100 nm and even more preferably in the order of 10 nm.

As detailed below in another set of embodiments, the shell of a microcapsule is functionalized with chemical moieties, polymers, groups or other molecules.

In one set of embodiments, the shell of a microcapsule may be hydrolyzed upon exposure to an enzyme.

In a more specific embodiment, when the shell comprises polyampholyte of proteinaceous origin, the protease enzyme may be employed to hydrolyze and/or destroy the shell.

### Some Aspects of the Microcapsule Core

In one aspect, the core of microcapsules is largely liquid and is preferably enriched in an inert hydrophilic compound belonging to the class of carbohydrates, oligosaccharides, polysaccharides, sugars and polymers that have multiple hydrophilic groups (e.g. hydroxy groups) such dextran, alginate, hyaluronic acid, glucan, glycogen, starch (amylose, amylopectin), agarose, agar-agar, heparin, pectin, cellulose, hemicellulose, chitosan, chitin, xanthan gum, curdlan, pullulan, inulin, graminan, levan, carrageenans, polyglycerols, and/or the modifications of said polyhydroxy substances, and/or any combination thereof. The liquid core may be slightly viscous and is preferentially aqueous. The aqueous is meant a composition providing the property of solubilizing the polar biochemical compounds.

The liquid core may be slightly hydrophobic, where the hydrophobic is meant a composition providing the property of solubilizing the apolar biochemical compounds such as fats or lipids.

In one set of embodiments, the hydrophilic polymer constituting the inner core of microcapsule is chosen at the concentration (w/v) in the range of 0.1-50% and more preferably at 1-20%.

In a more specific embodiment the hydrophilic polymer in the inner core of a microcapsule is composed of dextran or cellulose.

In a more specific embodiment the inner core of a microcapsule comprises salts and preferably kosmotropic salts at concentration higher than 1 μM and more preferably higher than 1 mM.

In one aspect, the viscosity of the inner core of the microcapsule may be reduced by treating the microcapsule with an enzyme (e.g. hydrolase).

In one set of embodiments, the polyhydroxy compound constituting the inner core of the microcapsule may be hydrolyzed by enzymatic (e.g. using hydrolase enzyme) or chemical process (using inorganic acid).

In a more specific embodiment, when the polyhydroxy compound constituting the inner core of the microcapsule is dextran, the dextranase enzyme may be employed to hydrolyze the dextran. Likewise, when the polyhydroxy compound constituting the inner core of the microcapsule is cellulose, the cellulase enzyme may be employed to hydrolyze the cellulose, and so on.

In another set of embodiments, the inner core of the microcapsule may be converted into a hydrogel.

In a more specific embodiment, the inner core of the microcapsules may be converted into a hydrogel when the inner core comprises precursors of the microcapsule's shell. When this occurs the average pore size of the core must be higher than the average pore size of the shell.

In one set of embodiments, the core and/or shell of a microcapsule may contain solid particles, such as metal nanoparticles, mineral particles, polymer particles, or composite particles. The size of said particle is preferentially from 10 nm to 10 μm.

In one aspect the microcapsule contains a cell or more than one cell, whereas the said cell(s) can be maintained alive for extended periods of time, cultured and expanded in 3D cell culture.

In another set of embodiments, the microcapsule contains a cell or more than one cell, whereas the said cell(s) can be analyzed using standard molecular biology and biochemical assays.

In another set of embodiments, the microcapsule contains a cell or more than one cell, whereas the said cell(s) can be analyzed using fluorescence-based assays.

In another set of embodiments, the microcapsule contains a cell or more than one cell, whereas the said cell(s) can be analyzed using microscopy-based assays.

In another set of embodiments, the microcapsule contains a cell or more than one cell, whereas the said cell(s) can be analyzed using nucleic acid analysis-based assays.

In another set of embodiments, the microcapsule contains a cell or more than one cell, whereas the said cell(s) can be analyzed using nucleic acid sequencing-based assays.

In another set of embodiments, the microcapsule contains a cell or more than one cell, whereas the said cell(s) can be lysed and their inner content evaluated using standard molecular biology and/or biochemical assays.

In another set of embodiments, the inner core of the microcapsule is between 1 μm and 100 mm in size, and more preferably between 10 μm and 1000 μm.

In another set of embodiments, the inner core of the microcapsule has a spherical or non-spherical shape.

In one set of embodiments, the dynamic viscosity of liquid core is preferably in the range 0.1 to −100 cP (centipose) and more preferably in the range of 1.0 to 10 cP.

### Some Aspects Related to Producing the Water-In-Oil Droplet Having a Liquid Core and a Liquid Shell

In one set of embodiments the intermediate step in microcapsule generation involves production of water-in-oil droplets comprising the macromolecules (e.g., polyampholyte) that undergo liquid-liquid phase separation and may form aqueous two-phase system comprising an outer aqueous phase and an inner aqueous phase, where the outer aqueous phase totally envelopes the inner aqueous phase.

A further aspect of the disclosure reveals that two co-existing aqueous phases in water-in-oil droplets may preferentially phase separate into an outer phase (liquid shell) and an inner phase (liquid core).

In one set of embodiments of the disclosure reveals that liquid shell and liquid core formation may occur upon a difference in solvent affinity sufficient to induce phase separation.

In one set of embodiments the liquid shell and liquid core formation may be enhanced by temperature, salts, coacervation.

In another set of embodiments of the disclosure reveals that upon liquid-liquid phase separation the liquid shell (liquid film) totally envelopes the liquid core.

In one set of embodiments the polyampholyte constitutes the precursors of microcapsule's shell and exhibits a phase-transition behavior in an aqueous solution (e.g., water).

In one set of embodiments macromolecules constituting the microcapsule (e.g., polyampholyte and polyhydroxy compound) undergo phase separation or coacervation inside water-in-oil droplets.

In one set of embodiments the liquid-liquid phase separation macromolecules constituting the microcapsule (e.g.,

Another aspect of the disclosure reveals that upon liquid-liquid phase separation the polyampholyte constituting the microcapsule may form a liquid coacervate film (liquid shell) entirely enveloping a liquid core comprising a dilute phase of the same polyampholyte, and where the dilute phase may also comprise polyhydroxy compound, and/or salts.

In one set of embodiments the polyhydroxy compound and/or salt added to the polyampholyte solution may facilitate the coacervation of the polyampholyte constituting the liquid shell, when both the polyampholyte and polyhydroxy compound and/or salt compound are mixed together.

In one set of embodiments, the polyampholyte may be mixed with other macromolecule such as polyhydroxy compound (e.g., dextran) and allowed to phase separate into a liquid phase enriched in a polyampholyte and another liquid phase enriched in a macromolecule.

In one set of embodiments the macromolecule constituting the liquid core (e.g., polyhydroxy compound) may facilitate the coacervation of the polyampholyte constituting the liquid shell, when both the polyampholyte and macromolecule are mixed together.

In one set of embodiments of the disclosure reveals that water-in-oil droplets the containing polyampholyte and polyhydroxy compound may form two aqueous phases with a shared solvent.

In one set of embodiments, upon liquid-liquid phase separation, the polyampholyte and polyhydroxy compound may be unevenly distributed between the two phases (liquid shell and liquid core).

In one set of embodiments, the dynamic viscosity of liquid shell and liquid core is preferably in the range 0.1 to −100 cP (centipose) and more preferably in the range of 1.0 to 10 cP.

In another embodiment, the liquid-liquid phase separation and gelation may occur simultaneously.

In another embodiment, the liquid-liquid phase separation and gelation of liquid phase may occur simultaneously, whereas preferably only one liquid phase (e.g., liquid shell) forms a gel.

In one more specific embodiment, the water-in-oil droplets may be produced in a microfluidic system comprising:
an inlet for continuous phase (carrier oil) supplemented with a surfactant;
an inlet for the first aqueous fluid comprising the precursors of the microcapsule's shell;
one or several inlets for the aqueous fluid(s) comprising polyhydroxy compound(s) and/or cells;
a microchannel combining the aqueous fluids;
the flow focusing junction where carrier oil meets the aqueous fluid(s);
the channel for droplet stabilization by surfactant supplemented in the carrier oil, and;
the water-in-oil droplet collection outlet.

In another aspect, the droplets may be of different size, ranging from 10 μm to 100 mm and more preferably in the range of 50-1000 μm.

In another aspect, by controlling the size of the microfluidics channels, the geometry of the flow focusing junction and the speed at which the aqueous phase(s) and the carrier oil are introduced into a water-in-oil droplet generation device, the droplet size may be precisely controlled.

In a set of embodiments, the shell precursor (polyampholyte), polyhydroxy compound, and cells, are emulsified with a carrier oil on a water-in-oil droplet generation device (e.g., microfluidics chip).

In a more specific embodiment, one aqueous phase containing the polyampholyte is brought in contact with a second aqueous phase containing the polyhydroxy compound and cells, and then brought into contact with the carrier oil. When aqueous phase(s) meet the carrier oil the water-in-oil droplets are formed at the flow focusing junction, or downstream the flow focusing junction.

In a more specific embodiment the microcapsule is generated using a microfluidics device, however, other type of devices, chips, systems and assemblies that generate liquid droplets are suitable for producing the said microcapsule. Such devices, systems and assemblies have been reported previously [18, 24, 25].

In a particular embodiment, the carrier oil used to generate droplets comprised a fluorinated oil and a fluorosurfactant. In a more specific embodiment, the fluorinated oil is HFE-7500 fluid. In another specific embodiment, the fluorosurfactant is PFPE-PEG-PFPE (perfluoropolyether-polyethylene glycol-perfluoropolyether) tri-block copolymer. In some particular embodiments, the carrier oil used to generate droplets is a fluorinated oil and comprises PEG-PFPE (polyethylene glycol-perfluoropolyether) di-block copolymer. Said surfactant(s) being present in the carrier oil at a concentration ranging from 0.05% to 10% (w/w), preferably ranging from 0.1% to 5% (w/w), more preferably ranging from 1% to 4% (w/w). The method of the present invention is not limited by the type of surfactant or the carrier oil used. One of ordinary skill in the art will be able to select the appropriate surfactant and carrier oil.

In an embodiment, the carrier oil is selected from the group consisting of fluorinated oil (fluid) such as FC40 oil (3M®), FC43 (3M®), FC77 oil (3M®), FC72 (3M®), FC84 (3M®), FC70 (3M®), HFE-7500 (3M®), HFE-7100 (3M®), perfluorohexane, perfluorooctane, perfluorodecane, Galden-HT135 oil (Solvay Solexis), Galden-HT170 oil (Solvay Solexis), Galden-HT110 oil (Solvay Solexis), Galden-HT90 oil (Solvay Solexis), Galden-HT70 oil (Solvay Solexis), Galden PFPE liquids, Galden® SV Fluids or H-Galden® ZV Fluids; and hydrocarbon oils such as Mineral oils, Light mineral oil, Adepsine oil, Albolene, Cable oil, Baby Oil, Drakeol, Electrical Insulating Oil, Heat-treating oil, Hydraulic oil, Lignite oil, Liquid paraffin, Mineral Seal Oil, Paraffin oil, Petroleum, Technical oil, White oil, Silicone oils or Vegetable oils.

### Some Aspects of Producing the Intermediate and Final Microcapsule

In one aspect the intermediate-microcapsule is formed when the liquid shell (liquid film) enveloping the liquid core is gelled. When the liquid shell forms a gel, the intermediate microcapsule is formed.

In one set of embodiments, when the liquid shell is gelled, the liquid core may also form a gel, when this occurs an intermediate microcapsule with a hydrogel core is formed.

In some embodiments, during the transition from liquid state to the gelled state, the individual precursors (monomers, polymers) of the liquid shell join and form a non-covalently cross-linked gel (solidified shell).

In some cases, the precursors may form a solidified shell either contemporaneously (during liquid-liquid phase separation) or sequentially (after liquid-liquid phase separation).

In some cases, the formation of the intermediate-microcapsule may occur without a clear pronounced liquid shell (liquid film) formation. In such case, the precursors constituting the solidified shell may be continuously deposited onto the outer phase (shell, film), during the formation of a liquid core by polyhydroxy compound.

One aspect of the disclosure reveals that the solidified shell comprises the precursors that may be covalently cross-linked upon activation by photo-initiator and/or irradiation and/or chemical agent, or any combination thereof. When the solidified shell of intermediate-microcapsule is covalently cross-linked the final microcapsule of this invention is formed, hereinafter for simplicity referred as microcapsule.

In one set of embodiments the precursor is a macromolecule (polyampholyte, polyelectrolyte or polymer).

In one set of embodiments, the intermediate-microcapsule is converted to a final-microcapsule while the intermediate-microcapsule is suspended in aqueous solution.

In another set of embodiments, the intermediate-microcapsule is converted to a final-microcapsule while the intermediate-microcapsule remains surrounded by the carrier oil containing surfactant.

In one set of embodiments the intermediate-microcapsule is formed upon temperature change.

In a specific embodiment, the solidification of the outer shell can be achieved by temperature-induced physical gelation of the proteinaceous shell.

In a more specific embodiment the proteinaceous shell is largely composed of polyampholyte belonging to extracellular matrix proteins or its hydrolyzed form, such as gelatin.

In another set of embodiments, the intermediate-microcapsule is formed upon coacervation.

One aspect of the disclosure reveals that temperature induced gelation leads to a reversible formation of intermolecular bonds between the individual monomers (polyampholyte chains) constituting the intermediate-microcapsule's shell.

In one specific embodiment, the intermediate-microcapsule's shell forms a thermo-reversible gel.

In another specific embodiment, the intermediate-microcapsule's shell forms a physically cross-linked gel (solidified gel).

In set of embodiments of the disclosure, the intermediate-microcapsule comprises a liquid core.

In another set of embodiments, the plurality of water-in-oil droplets comprising two aqueous phases (often referred as aqueous two-phase system) may be converted to the intermediate microcapsule by solidifying the outer phase (shell) of the said droplet.

In another set of embodiments, coexisting liquid phases (e.g., forming a liquid core and a liquid shell) are achieved inside droplets under the shared solution conditions.

In one set of embodiments the intermediate-microcapsules are released into aqueous environment by bursting (breaking) emulsion droplets and/or water-in-oil droplets.

In one set of embodiments the intermediate-microcapsule remains intact and does not burst when suspended in aqueous suspension.

In another set of embodiments, the intermediate-microcapsule not only remains intact but also retains encapsulated species (e.g. cells) when suspended in aqueous suspension.

In another set of embodiments, the intermediate-microcapsule remains dispersed in the carrier oil with or without a surfactant.

Some aspects of the disclosure reveal that intermediate-microcapsules can be subjected to multistep laboratory procedures such as pipetting, centrifugation, etc., and still retain encapsulated species (e.g., cells, nucleic acids).

Some aspects of the disclosure reveal also reveal that the intermediate-microcapsule may be dispersed in aqueous buffers of different composition, and still remain intact and retain encapsulated species.

In another specific embodiment, the intermediate-microcapsules may be dispersed in an aqueous environment at required temperature and still retain encapsulated cells and/or biomolecules (e.g. nucleic acids).

In one specific embodiment it may be preferable to handle the intermediate-microcapsule at room temperature and below, and more preferably below 12° C.

In one set of embodiments the shell of intermediate-microcapsule comprises the precursors that form covalent bonds upon a reaction with a chemical agent, upon irradiation or upon enzymatic reaction.

In another set of embodiments, the shell of intermediate-microcapsule preferentially comprises the precursors that form covalent bonds upon a reaction with a photo-initiator.

In another set of embodiments, upon the polymerization, the solidified shell of the intermediate-microcapsule forms a chemically (covalently) cross-linked gel.

In another specific embodiment, the solidified shell may be further stabilized by chemical cross-linking (e.g. using photo-polymerization).

The intermediate-microcapsules may be further treated with chemical agent and/or light to create a temperature-resistance shell, for example, by triggering chemical crosslinking of a shell via photo-polymerization.

In one specific non-limiting embodiment the precursor (monomer) of a polymerized shell is gelatin derivative (e.g., gelatin methacrylate).

In one set of embodiments, the concentration of monomers (e.g., gelatin methacrylate) is chosen at the concentration (w/w, weight/weight or w/v, weight/volume) in the range of 0.1-20% and more preferably at 1-10% and even more preferably between 2-6%.

In another set of embodiments, the degree of substitution of the precursor (e.g., gelatin methacrylate) is preferably higher than 10% but lower than 99%, and more preferably in the range of 20 to 90%, and even more preferably in the range from 60 to 80%.

In another set of embodiments, the cross-linker moiety density and/or monomer amount in the shell allows tuning the mechanical properties of microcapsules.

In one set of embodiments the cross-linking of the intermediate-microcapsule is performed by supplying the chemical reagent (e.g., cross-linking agent, photo-initiator, catalyst) externally. Where “externally” means through the solution in which the intermediate-microcapsules are dispersed.

In another set of embodiments, when the intermediate-microcapsule remains surrounded by the carrier oil containing surfactant (or without a surfactant), the final-microcapsule is produced by supplying the cross-linking agent, chemical agent, photo-initiator or the catalyst through the same carrier oil in which the intermediate-microcapsule remains dispersed.

In another set of embodiments, the intermediate-microcapsule may be converted to a final-microcapsule by supplying the cross-linking agent, photo-initiator or catalyst through the carrier oil with or without a surfactant.

In one set of embodiments the cross-linking of the intermediate-microcapsule is catalyzed by irradiation.

In one specific embodiment the intermediate-microcapsule is converted to final-microcapsule by supplying the photo-initiator externally and activating the said photo-initiator with a light.

In one set of embodiments the cross-linking of the intermediate-microcapsule is catalyzed by supplying the photo-initiator belonging but not limited to Type I (Norish Type I) and Type II (Norish Type II) initiators or Amine synergists.

In a set of embodiments, the intermediate-microcapsule may be converted to a final-microcapsule using a variety of cross-linking agents some of which, but are not limited, may include glutaraldehyde, glyceraldehyde, genipin, carbodiimides, N-hydroxysuccinimide, diisocyanates, poly(epoxy)-compounds, acyl azides, amine-crosslinking agents (e.g., dithiobis(succinimidylpropionate)), etc.

In another set of embodiments, the intermediate-microcapsule may be converted to a final-microcapsule using radical polymerization reaction initiated by chemical agents such TEMED and APS, redox initiators (e.g., Irgacure 2959), etc.

In another set of embodiments, the intermediate-microcapsule may be converted to a final-microcapsule using enzymatic reaction (e.g., using aminotransferases such as transglutaminase to cross-link lysine to glutamine residues).

In a more specific embodiment the intermediate microcapsule is converted to a final-microcapsule via photo-polymerization reaction.

In a set of embodiments, when the final-microcapsule is formed, the thickness of the cross-linked shell of the said microcapsule is between 0.2 μm to 100 μm, and preferably in the range of 1 μm to 20 μm thick.

In a set of embodiments, the shell thickness of the intermediate microcapsule is between 0.2 μm to 100 μm, and preferably in the range of 1 μm to 20 μm thick.

In one specific embodiment the microcapsule is formed by cross-linking the modified gelatin monomers into elastic and thermostable gelatin polymer mesh.

In another specific embodiment, the microcapsule having a cross-linked shell may be dispersed in aqueous environment and retain biological encapsulated cells for a sufficiently long period of time that may be required to culture, harvest and/or analyze the cells.

In another specific embodiment, the microcapsule having a cross-linked shell may be dispersed in aqueous environment and retain all, or a portion of the encapsulated species (e.g. nucleic acids, biomolecules).

In one aspect, the microcapsules are produced by following a general procedure:
i) Injection of the first aqueous fluid comprising the polyampholyte in a water-in-oil droplet generation device;
ii) Injection of the second and/or other aqueous fluid(s) comprising polyhydroxy compound(s) and/or cells in a water-in-oil droplet generation device;
iii) Forming the water-in-oil droplets comprising the polyampholyte, the polyhydroxy compound, and the cells;
iv) Providing sufficient time for the outer liquid shell and inner liquid core to form inside the water-in-oil droplets whereas the outer liquid shell may be enriched in the polyampholyte, and the inner liquid core may be enriched in the polyhydroxy compound; and where the cells are preferentially distributed in the liquid core;
v) Temperature induced gelation of the liquid shell;
vi) Formation of the intermediate microcapsule with a solidified shell;
vii)Breaking the emulsion droplets to release the intermediate microcapsules having a solidified shell;
viii) Further stabilization of the intermediate microcapsule by covalently cross-linking the shell;

### Some Aspects of Functionalizing the Microcapsules

In another set of embodiments, the cross-linked shell of a final-microcapsule (herein a microcapsule) may contain the peptide(s) those amino acid sequence comprises Arg-Gly-Asx, Gly-Arg-Gly-Asx-Tyr, Gly-Arg-Gly-Asx-Ser, Tyr-Ile-Gly-Ser-Arg, Gly-Tyr-Ile-Gly-Ser-Arg-Gly, Ile-Lys-Val-Ala-Val, Lys-Arg-Glx, Arg-Glx-Asx-Val, Gly-Arg-Glx-Asx-Val-Tyr, Leu-Gly-Thr-Ile-Pro-Gly, Pro-Asx-Ser-Gly-Arg, Arg-Asx-Ile-Ala-Glu-Ile-Ile-Lys-Asx-Ala, Asp-Gly-Glx-Ala, Val-Thr-X-Gly, Val-Gly-Val-Ala-Pro-Gly or X-B-B-X-B-X, or any combination thereof. Where X is a hydrophobic amino acid, and B is positively charged basic amino acid, namely arginine or lysine.

In another set of embodiments, biomolecules (proteins, peptides, lipids, sugars) and/or entities such as oligonucleotides (e.g., DNA or RNA primers, nucleic acid fragments) may be incorporated into the microcapsule during cross-linking (polymerization) reaction.

In another set of embodiments, the incorporation of biomolecules (e.g., oligonucleotides, peptides) may be achieved via covalent or non-covalent association with the microcapsule shell or the microcapsule core, or combination thereof.

In another set of embodiments, the biomolecules and/or oligonucleotides may be incorporated via acrydite moiety, disulfide bond that becomes cross-linked to the microcapsule during the polymerization reaction. The incorporation of biomolecules and/or entities such as oligonucleotides may be achieved either during microcapsule formation, when the intermediate-microcapsule is formed, or when final-microcapsule is formed, following formation, or any combination thereof.

In another set of embodiments, the biomolecules (polyampholytes, polyelectrolyte, polymers, monomers) having covalently attached entities (e.g., the oligonucleotides, peptides, lipids, sugars) may be incorporated into the microcapsule's shell and/or core during the formation of the intermediate-microcapsule, or it may be incorporated into the microcapsule's shell and/or core after the intermediate-microcapsule is produced.

In another set of embodiments, the microcapsule may comprise a ligand (capture probe) capable of binding (capturing) biomolecule(s). The capture probe may comprise an antibody, antibody fragment, receptor, protein, oligopeptide, peptide, amino acids, enzyme cofactors, vitamins, small biochemical molecules or any other species capable of interacting with biomolecules on the surface of the cells, or intracellular biomolecules of the cells.

### Some Features of the Microcapsules

In one set of embodiments, the microcapsule is generated by producing water-in-oil droplet whereas the said droplet may serve as a soft and deformable mold, or template droplet, for the formation of a desirable size microcapsule. For example, the larger the droplet, the larger the final-microcapsule.

In one aspect, the disclosed microcapsules show high circularity and high concentricity. As defined above in the present disclosure the microcapsule has circularity C=0.94±0.06 or in some cases C=0.9±0.1, and/or the relatively high concentricity, O≥66%.

In another aspect, the microcapsule may comprise a very thin shell (1-4 μm thick) and still support mechanical integrity and retention of the encapsulated cell, while microcapsule is swelled or expanded more than 30%.

In another aspect, the said microcapsule's shell is permeable to low molecular weight molecules and compounds that may diffuse in and from the microcapsule. For example, microcapsule's shell may be permeable to compounds, reagents, molecules having molecular weight smaller than approximately 1000 Da, 2000 Da, 3000 Da, 5000 Da, 10.000 Da, 20.000 Da, 30.000 Da, 50.000 Da, 100.000 Da, 200.000 Da, 300.000 Da or 500.000 Da.

In another set of embodiments, the said microcapsule's shell prevents larger biomolecules from entering, or leaving, the microcapsule. For example, microcapsule's shell may prevent biochemical compounds, reagents and molecules having molecular weight larger than approximately 10.000 Da, 20.000 Da, 30.000 Da, 50.000 Da, 100.000 Da, 200.000 Da, 300.000 Da, 500.000 Da or 1000.000 Da from entering and leaving the microcapsule.

It should be understood for experienced in the field that the porosity and thus permeability of the microcapsule's shell may be altered in multiple ways of which non-limiting examples include adjusting the concentration of the precursors (e.g., polyampholyte) constituting the shell, adjusting the number of cross-linking sites in the shell, adjusting the number of cross-linking moieties (substitutions) on the precursors, altering polymerization conditions, and/or altering the composition of the shell with additives (e.g., adding PEG, polymers, proteins, polysaccharides, salts, etc.).

In another set of embodiments, the composition and solvent of the inner core of the microcapsule may be altered, modified or changed by suspending the said microcapsule in a solution having a desirable biochemical composition, and allowing the molecules from the said solution to transverse the shell and by doing so alter, modify or change the inner content of the microcapsule.

In another set of embodiments, the solvent and/or low molecular weight compounds occupying the core of microcapsule may be removed from the core by squeezing (pinching) the capsules without breaking the cross-linked shell.

### Some Aspects of Encapsulating, Culturing and Analyzing Cells

In one aspect, the microcapsule contains a cell or more than one cell, whereas the said cell(s) can be cultured and/or analyzed, for example, using microscopy, flow cytometry, standard molecular and cell biology techniques.

In one set of embodiments the microcapsules carrying the cells of interest are generated by first encapsulating the cells into liquid droplets and then converting the liquid droplets into microcapsules having a cross-linked shell. It should be understood that the liquid droplets may be water-in-oil droplets or it could be water-in-air droplets. In a preferred scenario the liquid droplets are water-in-oil droplets.

In another set of embodiments, the microcapsule contains a biological entity or a collection of biological entities (e.g., nucleic acids, proteins, biomolecules) that can be analyzed and/or amplified and/or barcoded, for example, but not limited to using standard molecular and cell biology techniques (e.g., RT, PCR, RT-PCR, sequencing etc.), microscopy, flow cytometry, etc.

Encapsulation of cells and/or biological species and/or reagents may be performed using a microfluidics device, capillary assembly or droplet generation device.

In another set of embodiments, a microcapsule provides a biocompatible compartment for an in vitro culture and harvesting of encapsulated cells.

In another set of embodiments, a microcapsule provides a biocompatible compartment for maintaining and/or analyzing the encapsulated cells and their physiological functions (e.g., growth, shape, division, metabolic activity, etc.).

In another set of embodiments, a microcapsule provides a biocompatible compartment for maintaining and/or analyzing the 3D cell structures, such as spheroids, organoids, tumoroids, tissues and other cell masses.

In a more specific embodiment the individual cells may be encapsulated in microcapsules and cultured for extended periods of time to allow their growth and generation of the daughter cells.

In another set of embodiments, the individual cells may be encapsulated in microcapsules and cultured for extended periods of time to into a clonal population of cells, and/or masses of cells, including but not limited to spheroids, organoids, tumoroids, tissues and other types of 3D cell assemblies (3D cell structures).

In another embodiment, the microcapsules carrying two, three or more cells can be incubated in suitable cell culture conditions to enable formation of spheroids, organoids, tumoroids, tissues and other types of 3D cell structures.

In another set of embodiments, multiple cells may be encapsulated in microcapsules and cultured for extended periods of time to generate diverse populations of cells, and/or masses of cells, including but not limited to spheroids, organoids, tumoroids, tissues and other types of 3D cell structures.

In another set of embodiments, the microcapsules carrying encapsulated cells can be added to a solution having cryoprotectant (e.g., methanol, acetate, dimethylsulfoxyde (DMSO), glycerol, trehalose, glycol, etc.,) and stored at liquid nitrogen for extended periods of time.

In another set of embodiments, the semi-permeability of the microcapsule's shell ensures that encapsulated cells may receive the nutrients from the cell growth media surrounding the microcapsule.

In another set of embodiments, the said microcapsule's shell is permeable to low molecular weight molecules and compounds that may diffuse in and from the microcapsule.

For example, microcapsule's shell may be permeable to nutrients and compounds having molecular weight smaller than approximately 1000 Da, 2000 Da, 3000 Da, 5000 Da, 10.000 Da, 20.000 Da, 30.000 Da, 50.000 Da, 100.000 Da, 200.000 Da, 300.000 Da or 500.000 Da.

In another set of embodiments, the said microcapsule's shell prevents larger biomolecules from entering, or leaving, the microcapsule. For example, microcapsule's shell may prevent nutrients, molecules and compounds having molecular weight larger than approximately 10.000 Da, 20.000 Da, 30.000 Da, 50.000 Da, 100.000 Da, 200.000 Da, 300.000 Da, 500.000 Da or 1000.000 Da from entering and leaving the microcapsule.

In another set of embodiments, the phenotype and/or genotype of the encapsulated cell(s) may be evaluated using physical or chemical techniques, molecular biology and biochemistry techniques known to a skilled person in the art. Whereas the phenotype is a cellular trace that can be measured or evaluated by fluorescence readout, microscopy readout, absorbance readout, enzymatic, regulatory or binding activity readout, metabolic activity readout, or other, and whereas the genotype is a trace measured or evaluated by nucleic acid analysis technique (e.g. PCR, RT-PCR, sequencing, fluorescence measurement, hybridization, absorbance, etc.).

In a specific set of embodiments, the microcapsules carrying encapsulated single-cell or population of cells may be subjected to multistep analytical procedures and yet still retain the encapsulated cell(s), the non-limiting examples of such procedures include flow cytometry, FACS, pipetting, centrifugation, analytical reactions, molecular biology assays, etc.,

In one set of embodiments, the encapsulated cells are preferentially distributed (retained) within the inner core of the microcapsules.

In one set of embodiments, the encapsulated cell and/or cells may attach to the inner interface of the shell and form monolayers, multilayers or other complex cell structures.

In another set of embodiments, a microcapsule provides a support (substrate) for encapsulated cells to attach. For example, the proteinaceous shell of the microcapsule may serve as a substrate for adhesion of encapsulated cells.

In another set of embodiments, the shell of the microcapsule may provide a substrate for cells to attach to the outer surface of the said shell. For example, the microcapsules may be suspended in a solution having cells and allowing the said cells to attach at the microcapsule' surface.

In another set of embodiments, a microcapsule provides a biocompatible compartment for cell co-culture of two or more cell types.

In another set of embodiments, the plurality of microcapsules carrying one type of cells can be mixed (suspended) with plurality other type of microcapsules carrying encapsulated cell(s) of different type; and allowing both cell types to communicate biochemically (e.g., via secreted biomolecules), yet remain physically separated from each other by the compartments.

In another set of embodiments, the plurality of microcapsules carrying cells of one type may be suspended in a solution (suspension) having a different type of cells and allowing encapsulated cells to biochemically communicate with cells in a suspension; whereas the encapsulated cells and the cells in a suspension are maintained physically separated from one another by the microcapsule's shell.

In another specific embodiment, the cells in a suspension might attach to the outer surface of the microcapsule, but are still maintained physically separated from the encapsulated cells by the microcapsule's shell.

In one set of embodiments, the present invention also relates to a microcapsule as a biocompatible compartment for co-encapsulation of different cell types in the same microcapsule in order to allow two cell types to interact biochemically with each other (e.g., through secreted biomolecules).

In another set of embodiments, the present invention also relates to a microcapsule as a biocompatible compartment for co-encapsulation of different cell types in the same microcapsule in order to allow two cell types to interact physically and establish cell-to-cell contact.

In another set of specific embodiments, a microcapsule provides a biocompatible compartment for performing a screening assay that is generally known as cell cytotoxicity assay [35].

In another set of specific embodiments, a microcapsule provides a biocompatible compartment for performing two-cell binding assay where the cancer cells and immune cells are interacting via soluble factors (e.g. secretion of antibodies against cancer cell), and/or interact physically by establishing cell-cell contact, within the same microcapsule.

In another set of specific embodiments, a microcapsule provides a biocompatible compartment for performing two-cell binding assay where the T-cell and target cell interacts via T-cell receptor.

In another set of specific embodiments, a microcapsule provides a biocompatible compartment for performing two-cell binding assay where the dendritic and target cell interacts via cell receptor(s).

In another set of specific embodiments, a microcapsule provides a biocompatible compartment for performing two-cell interaction assay where the natural killer (NK-cell) and target cells are interacting biochemically and/or physically, within the same microcapsule.

In one set of embodiments, the microcapsules dispersed in aqueous environment may be subjected to one, or more analytical or experimental treatments (e.g. microcapsules can be washed and dispersed in growth medium, or mixed with immunoassay reagents, etc.) in order to perform biological assay on encapsulated cells, or culture encapsulated cells.

In some aspects, a cell-based assay could be antibody binding assay, ELISA, cell viability assay, an assay for evaluation of cell metabolic function, protein synthesis assay, fluorescence staining, and other cell-based and molecular assays that are commonly used to obtain the phenotype or genotype information of the encapsulated cells.

In some aspects, an enzymatic assay can be reverse transcription (RT), polymerase chain reaction (PCR), RT-PCR and other nucleic acid amplification method to obtain genetic make-up information or genome-encoded information about the encapsulated cells.

In one set of embodiments, the microcapsules can be analyzed using a variety of laboratory techniques available in a research laboratory such as fluorescence and bright field microscopy, flow cytometer, FACS, dialysis, incubation at desirable temperature, etc.

In one set of embodiments, the microcapsule can be disintegrated to release the encapsulated cells and/or biomaterial.

In a specific embodiment, the microcapsule can be dissolved by enzymatic treatment such as protease or hydrolase, or combination thereof.

In one set of embodiments, the encapsulated cell(s) as well as complex 3D cell structures (e.g. spheroids, tumoroids, tissues) can be released by treating the capsules with enzyme (e.g., protease, trypsin).

The following are intended as examples only and do not limit the present disclosure.

### EXAMPLES

### Example 1: Validation of PEGDA/Dextran Capsules for Mammalian Cell Encapsulation and Retention

We have recently disclosed a method for producing the semi-permeable capsules composed of the outer shell composed of the polyethylene glycol (PEG) hydrogel and inner core enriched in polysaccharide (U.S. patent application Ser. No. 16/934,045, published as US 2020/400538A) and [31]. The microcapsule's core is composed of dextran-rich solution and the microcapsule's shell is composed of polyethylene glycol diacrylate (PEGDA) that forms a solid and elastic shell. Since PEGDA monomers are partly soluble in dextran phase, upon the polymerization the microcapsule's core forms a covalently crosslinked hydrogel mesh, that cannot be converted back to a liquid form. In one aspect, the reported core-shell microcapsules were composed of two hydrogel layers; a weak hydrogel mesh constituting the inner core of a microcapsule and rich in dextran, and a strong hydrogel shell largely composed of PEG.

In the published work we showed examples of semi-permeable capsule use for genotypic and phenotypic analysis of individual bacterial cells by performing multi-step workflows on hundreds and thousands of cells simultaneously. However, capsules approaching ≥60 μm size were less easy to produce and they tended to lose the concentricity. These limitations may have some negative consequences in biological assays that rely on encapsulation and retention of cells, organisms, or other biological materials. In particular, the biological assays that rely on isolation of individual mammalian cells often rely on micro-compartments (e.g. water droplets) that are close to 50 μm in diameter, or beyond, thus making PEGDA-based capsules challenging to implement. Moreover, the release of encapsulated cells and/or biological species relied on alkaline treatment that may be detrimental to encapsulated cells (and may hydrolyze and/or degrade encapsulated biological species), which it may be preferable to avoid in certain applications. Therefore, the microcapsule and composition reported previously have limitations that may limit their utility in some applications, particularly in relation to encapsulation, culture, and analysis of mammalian cells Following the report published previously [31] we attempted to isolate the mammalian

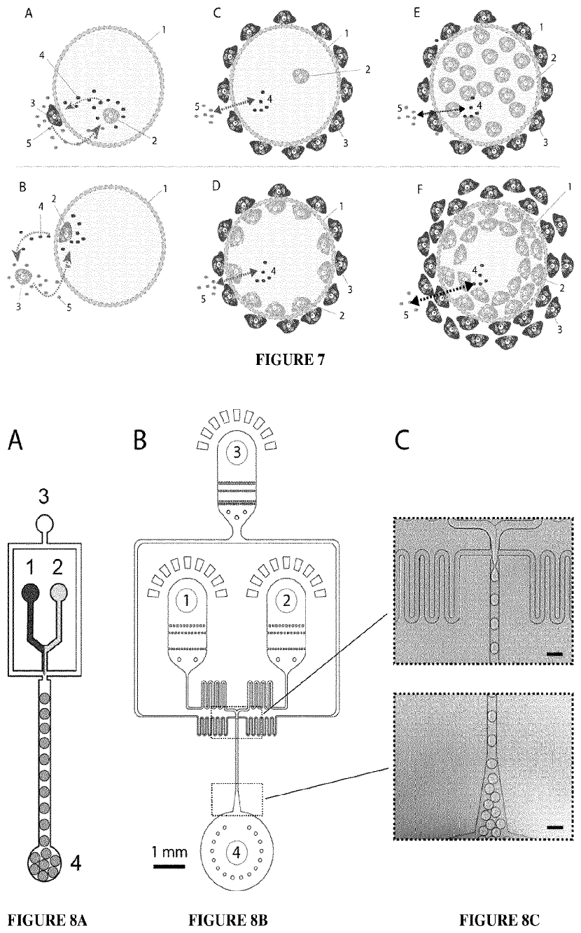

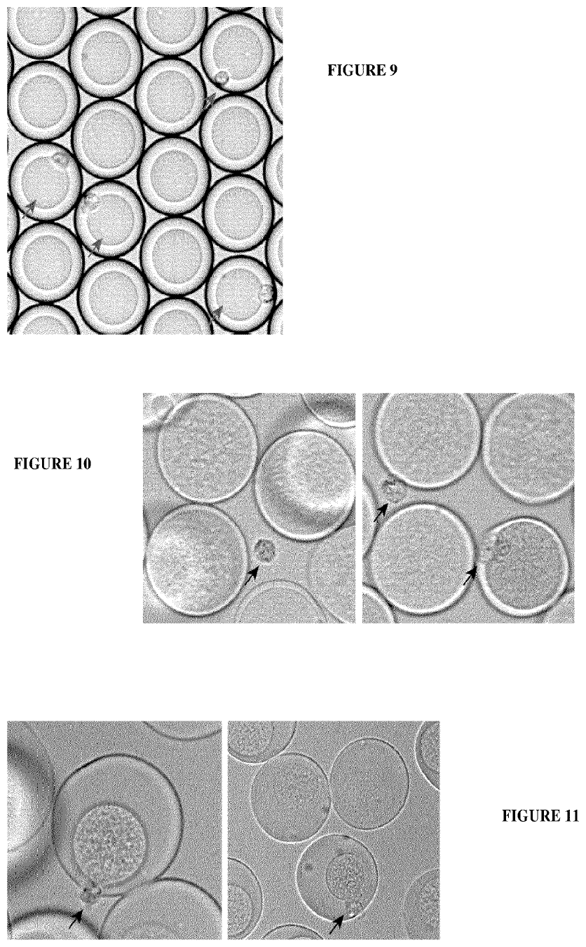

cells into a variety of capsules above 60 μm in size. The capsules were composed of PEGDA/dextran blend and were generated using a microfluidic device depicted in FIG. 8A-C. We found that encapsulated mammalian cells preferentially moved to the PEGDA phase and/or arranged themselves at the PEGDA/Dextran interphase (FIG. 9). When mammalian cells were encapsulated in the PEGDA/Dextran capsules, with shells in the range of 4-10 μm thick, the cells tend to escape the compartmentalization (FIG. 10), resulting in a significant number of capsules void of cells. To improve the cell retention, the capsule shell was increased close to, or larger than, the size of the cell (˜20 μm). However, while increasing the shell thickness visibly reduced the number of prematurely released cells, yet a thicker shell did not prevent cells from entering the PEGDA phase (FIG. 11). More importantly, the capsules with a thicker shell lost the concentricity, which led to capsules with uneven shell thickness, which in turn caused cell escape through the thinner shell part (FIG. 11). Others in the field have also noticed that mammalian cells tend to move to PEG-rich phase when working with liquid ATPS droplets composed of PEG/dextran blend [30]. Therefore, our results as well as literature reports indicate that previously reported polymer composition of the microcapsules, while is suitable for isolation of small bacterial cell, it is less suitable for efficient encapsulation and retention of mammalian cells.

### Example 2: Generation of Microcapsules Using Irradiation (Photo-Illumination)

We postulated that a microcapsule having a polyampholyte shell could potentially provide superior cell encapsulation and retention as compared to PEGDA-based microcapsules reported earlier. In addition, a polyampholyte shell comprising a proteinaceous material could provide not only a physical barrier to separate cells from the external environment but also act as a solid and elastic substrate for encapsulated cells to adhere to. To produce microcapsules composed of a proteinaceous shell we first exemplify the use of gelatin derivative, a thermo-responsive protein, that solidifies at lower temperatures [77], and which can be chemically (covalently) cross-linked, when it carries appropriate chemical substitution (e.g., acrydite, methacryloyl, methacrylamide, methacrylate, thiol, etc.). The rheological properties of the gelatin-based hydrogels can be controlled by the degree of substitution, polymer concentration, initiator concentration, and UV irradiation conditions [77]. In another example presented below the microcapsules comprising a solidified shell composed of polyelectrolyte (a non-modified gelatin) were covalently cross-linked using an enzymatic reaction (e.g., transglutaminase), or a cross-linking agent (e.g., genipin). Other polyelectrolytes comprising a peptide bond, proteins, peptides, oligopeptides or polypeptides, including but not limited to collagen, elastin, elastin-like proteins, mucin, fibrin, laminin, gluten, gliadin, abductin, resilin, tropoelastin, fibronectin, silks proteins, vimentin, poly-L-lysine, the extra-cellular matrix oligopeptides, peptides or proteins, proteoglycans, glycosaminoglycans—or their hydrolyzed forms—may be applicable for the formation of the microcapsule shell.

To exemplify the production of microcapsules comprising a shell composed of polyampholyte derivative, we first generated water-in-oil droplets on a 40-μm deep co-flow microfluidics device (FIG. 8), by loading 3% (w/v) gelatin methacrylate (GMA) and 15% (w/v) dextran (MW ˜500k) solutions. In the experiments where the cell encapsulation was performed, the cells were suspended in dextran solution accordingly. Typical flow-rates used were: for gelatin methacrylate solution—250 μl/h; for dextran solution (with or without cells)—100 μl/h and for the carrier oil—700 μl/h. The carrier oil was HFE7500 oil supplemented with 1.5% (w/v) tri-block (PFPE-PEG-PFPE) fluorosurfactant, where PEG has molecular weight 600 g mol−1 and PFPE (perfluoropolyether) has molecular weight 6000 g mol−1 [62].

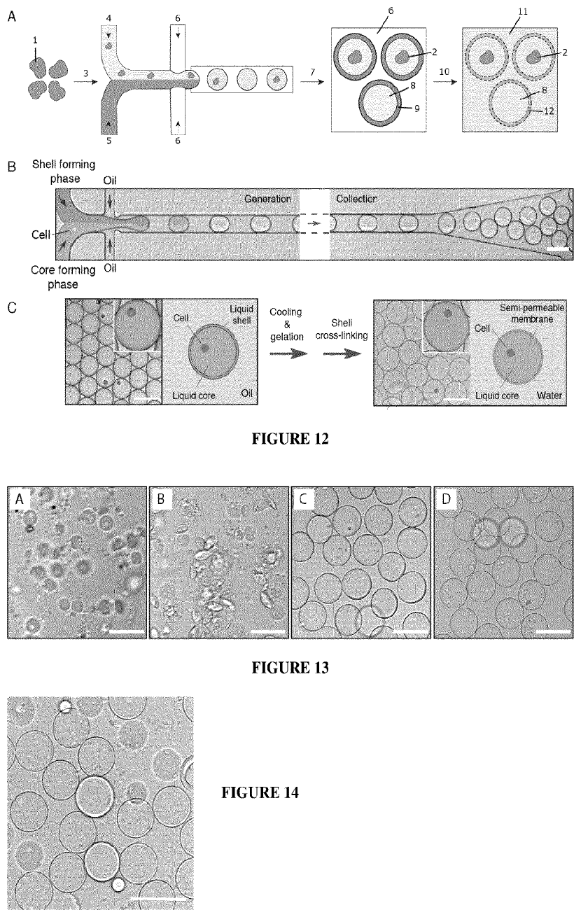

After emulsification step, the resulting water-in-oil droplets formed a core/shell structure comprising a liquid core enriched in dextran, and a shell enriched in GMA (FIG. 12B). The droplets were then subjected to a two-step polymerization procedure. At first, the water-in-oil droplets were incubated at selected temperature (˜4° C.) for −30 min to complete the physical gelation of GMA phase. The resulting microcapsules (called intermediate-microcapsules) comprising a solidified polyampholyte shell were recovered from the emulsion by breaking the emulsion with emulsion breaker (Droplet Genomics, DG-EB-1) and released into 1×PBS buffer supplemented with 0.1% (v/v) Pluronic F-68. The suspension with intermediate-microcapsules was then supplemented with photo-initiator 0.1% (w/v) lithium phenyl-2,4,6-trimethylbenzoylphosphinate (LAP) (Sigma-Aldrich, 900889-1G) and photo-polymerized under exposure to 405 nm light emitting diode (LED) device (Droplet Genomics, DG-BR-405) for 20 seconds. The resulting microcapsules contained chemically cross-linked polyampholyte shell (FIG. 12C). Following the aforementioned polymerization procedure, we could reproducibly generate the capsules with a clear, well-centered core enriched in polyhydroxy substance (dextran), and a solidified shell composed of covalently cross-linked polyelectrolyte.

As it was reported previously gelatin methacrylate (GMA) will form a hydrogel of different Young modules depending on the degree of substitution [77]. We therefore investigated the degree of substitution that is required to achieve stable and concentric microcapsules upon photo-polymerization. We tested microcapsule production by photo-polymerization using GMA with 0, 40, 60 and 80% degrees of substitution. For each test 3% (w/v) GMA with a given degree of substitution, and 15% (w/v) dextran (MW ˜500k) was used. Following aforementioned two-step polymerization procedure, we generated capsules and evaluated their quality under bright field microscope. Results presented in FIG. 13A-D show that GMA with 60 and 80% substitution ensured stable capsule generation, while GMA with 0-40% substitution failed to generate stable capsules. Increasing the GMA amount from 3% to 5% (w/v), while keeping dextran at 15% (w/v), the capsules could be generated with GMA having 40% degree of substitution (FIG. 14). Therefore, we conclude that production of stable microcapsules by photo-polymerization depends on both the GMA substitution degree, which preferably should be at or above 40%. In addition, we conclude that production of stable microcapsules by photo-polymerization also depends on the total amount of GMA used in the mix, which preferably should be at or above 3% (w/v). Noteworthy, capsule production also depends on GMA concentration, temperature, pH, salts, etc.

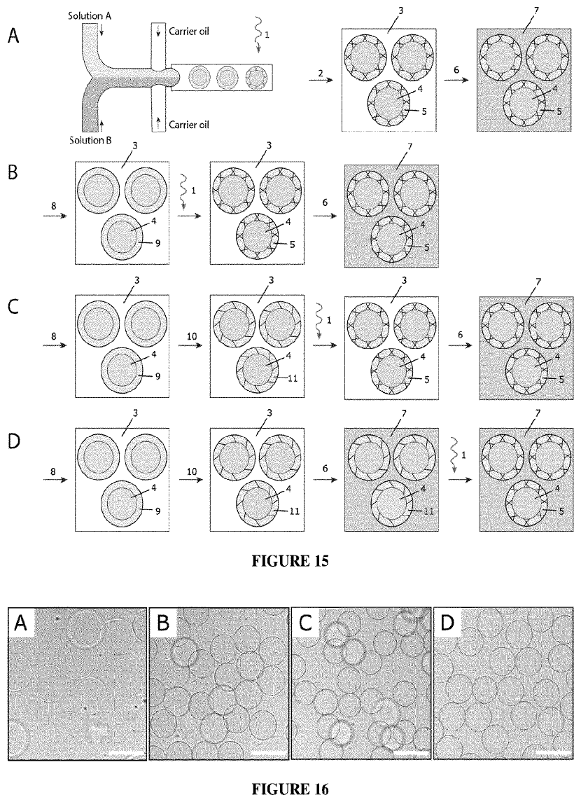

Indeed, it should be understood that there are multiple paths for producing capsules composed of proteinaceous shell and liquid core as well as capsules composed of proteinaceous shell and semi-liquid core, or capsules composed of proteinaceous shell and hydrogel core. Some of these approaches, but not limited to, have been verified experimentally and are schematically shown in FIG. 15. In a typical scenario, the water-in-oil droplets are generated and incubated/collected at a selected temperature that is needed to induce sol-gel transition of the shell phase. When using gelatin or gelatin derivatives solidification (gelation) and/or precipitation of the shell is typically completed after 30 min incubation at 4° C., however solidification and/or precipitation may also occur at higher temperatures (e.g., 22° C.). More generally, sol-gel transition of microcapsules disclosed here depends on depends on degree of substitution, chemical modifications, temperature, pH, salts, ionic strength of buffer. When sol-gel transition is achieved (as in the two-step procedure described above) the resulting solidified (gelled) capsules can be released into aqueous buffer (preferably at temperature that is below the shell melting temperature) and incubated for the extended period of time. The shell of the intermediate-microcapsules may be chemically cross-linked using photo-initiator, chemical agents, or enzymes into elastic of the proteinaceous hydrogel shell. Different variations of this methodology are possible and some are described below:
In one example (FIG. 15A), the shell of capsules can be chemically cross-linked following core/shell formation during water-in-oil droplet generation. By irradiating the emulsion droplets, the cross-linked microcapsules are formed. Resulting microcapsules dispersed in aqueous buffer (e.g. 1×PBS buffer) and imaged under bright field microscope are shown in FIG. 16A.
In another example (FIG. 15B), the shell of capsules can be polymerized by photo-illuminating (irradiating) the collected emulsion off-chip. In this scenario, more time is provided for the phase separation to occur (formation of a core and a shell formation) and therefore once the final microcapsules is formed, and dispersed in aqueous buffer, the microcapsules are slightly smaller size, as shown in FIG. 16B.
In yet another example (FIG. 15C), the shell of capsules can be polymerized in a two-step process such that microcapsules remain suspended in the carrier oil. Specifically, following emulsion collection off-chip, the shell of liquid droplets can be solidified by incubating droplets at a temperature below sol-gel transition point (e.g., 4° C.) followed by photo-illumination and release into aqueous buffer. Resulting capsules imaged under bright field microscope as shown in FIG. 16C.
In yet another example (FIG. 15D), the shell of capsules can be polymerized in a two-step process where at first the emulsion droplets are incubated at a temperature below the sol-gel transition point (e.g. 4° C.) to induce gelation of the shell. Next, resulting intermediate microcapsules are dispersed in aqueous phase (buffer) followed by a cross-linking reaction induced by photo-polymerization. The resulting capsules are shown in FIG. 16D.

### Example 3: Generation of Microcapsules Using Chemical Agents

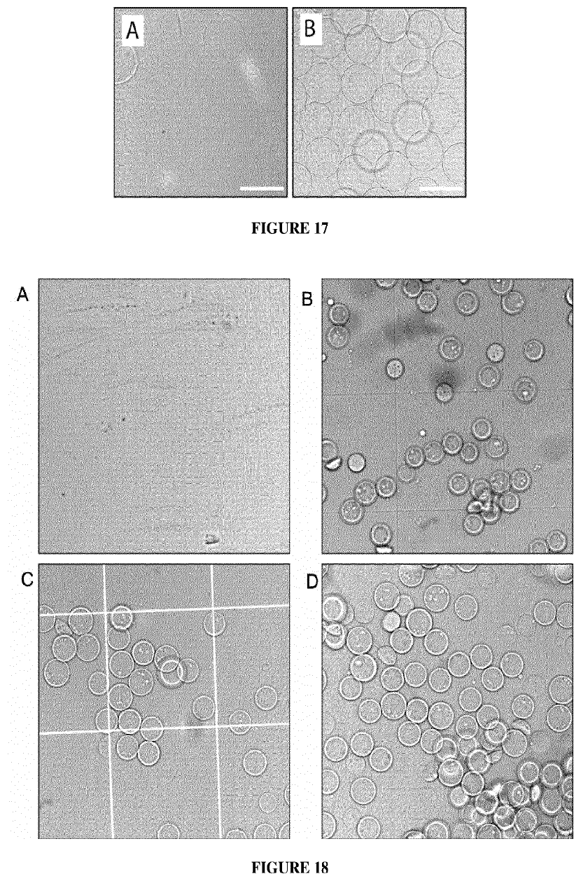

It should be understood that microcapsules of this disclosure can be generated using different means of cross-linking. In a separate example revealed in FIG. 17A the shell of microcapsules was cross-linked using a chemical agent. First, the water-in-oil droplets were generated by supplementing the GMA phase with 0.3% (w/v) Ammonium Persulfate (APS), while the carrier oil was supplemented with 0.4% (w/v) Tetramethylethylenediamine (TEMED). The emulsion was collected off-chip and incubated at room temperature for 2 hours to allow sufficient period of time for chemical agents, TEMED and APS, to initiate and complete the shell cross-linking reaction. The resulting capsules were suspended in aqueous buffer and evaluated microscopically as shown in FIG. 17A.

In yet another example revealed in FIG. 17B, the shell of capsules was polymerized (cross-linked) using a combination of physical and chemical means. The water-in-oil droplets comprising GMA and dextran phases were collected off-chip and incubated at 4° C. for 30 min to induce physical gelation of the shell. The solidified capsules were then resuspended in aqueous buffer (lx DPBS, 0.1% (w/v) F-68) containing 0.3% (w/v) APS, 0.4% (w/v) TEMED, and incubated at room temperature for 2h to induce chemical cross-linking of capsules' shell. The resulting capsules are shown in FIG. 17B.

### Example 4: Generation of Microcapsules Comprising a Non-Modified (Natural) Gelatin: Using a Cross-Linking Agent

A variety of chemical compounds may act as cross-linking agents. For example, when the polyampholyte that is to constitute the microcapsules' shell comprises a primary amine the shell can be formed by crosslinking using glutaraldehyde or other agents. Alternatively, when the polyampholyte comprises a carboxy group the shell can be formed by cross-linking using carbodiimides and other cross-linking agents. In a proof-of-concept example shown below the microcapsules' shell was made from a non-modified gelatin cross-linked using genipin (Sigma, cat no G4796).

First, the materials listed in Tables 1 were mixed and then centrifuged at ˜37-40° C.

TABLE 1
Composition of biphasic system, composed of and dextran

| | | |
|---|---|---|
| | Composition of biphasic system, | |
| Volume | Material | Final |

| | | | |
|---|---|---|---|
| 100 | μL | 10% (w/w) gelatin from porcine skin | 5% (w/v) |
| 40 | μL | 25% (w/w) dextran (MW 500K) | 5% (w/v) |
| 60 | μL | 1X PBS, pH [7.4] | — |
| 200 | μL | Final | |

After phase-separation the top layer was enriched in gelatin and the bottom was enriched in dextran. These phases were aspirated into separate tubes and incubated at 37-40° C. until the encapsulation was proceeded.

Water-in-oil droplets were generated using a microfluidics chip having 20 μm height microchannels, and a nozzle 20 μm wide. The flow-rates used were: gelatin-rich phase—100 μl/h, dextran-rich phase—100 μl/h and carrier oil—300 μl/h. Water-in-oil droplet generation was performed at room temperature (22° C.), while heating the microfluidics chip at 37° C. Emulsion droplets were collected for 20-25 minutes in 1.5 ml tube, prefilled with 200 μl of light mineral oil. Next, emulsions were transferred to 4° C. for 30-60 minutes to induce gelatin solidification and formation of an intermediate-microcapsule. Continuing procedures on ice, the intermediate-microcapsules were recovered from the emulsion using a commercial emulsion breaker (Droplet Genomics, DG-EB-1) and resuspended 1×PBS supplemented with 0.1% (w/v) Pluronic F-68. The intermediate-microcapsules were stored at 4° C. before processing them further in a cross-linking reaction.

To create a microcapsule comprising a covalently cross-linked shell the intermediate microcapsules were mixed with 0.5% (w/v) genipin and incubated at 4° C. for 1 and 24 hours. Following the incubation, the microcapsules were heated at 50° C. for 10 min. If the shell of microcapsule is successfully cross-linked, the capsules should become thermostable and remain intact, and if a cross-linking reaction is unsuccessful the microcapsules will melt or will end up defective. Results shown in FIG. 18A show that performing a cross-linking reaction with 0.5% genipin for 1h at 4° C. is not sufficient to obtain stable and intact microcapsules. However, performing a cross-linking reaction with 0.5% genipin for 24 h at 4° C. produced thermo-resistant microcapsules that remained intact after heating at 50° C. FIG. 18B. Indeed, a person experienced in the art will understand that the chemical cross-linking reaction time can be altered using higher concentrations of cross-linking agent, using higher temperature, or using a more reactive cross-linking agent (e.g. glutaraldehyde).

### Example 5: Generation of Microcapsules Comprising a Non-Modified (Natural) Gelatin: Using an Enzymatic Reaction

While it is convenient to use the chemical cross-linking reaction for generating the microcapsule in some circumstances it may be advantageous to use ‘milder” reaction conditions and/or “milder” reagents that would not be damaging to the encapsulated cells or biological species.

When using a polyampholyte comprising primary amine and amide residues the cross-linking reaction may be performed using enzymes catalyzing the formation of an isopeptide bond. In the following non-limiting example, the microcapsules were made from a polyampholyte having lysine and glutamine residues that were cross-linked by employing transglutaminase enzyme (Sigma, SAE0159-25UN).

The intermediate-microcapsules, comprising a gelatin extracted from porcine skin and 500K dextran were generated as described in Example 4. The intermediate-microcapsule comprising a solidified shell enriched in gelatin were mixed with 0.3 U/ml of microbial transglutaminase (mTG) and incubated at 4° C. for 1h, at 23° C. for 30 min in order to initiate the cross-linking (isopeptide bond formation) reaction. Following the incubation, the microcapsules were heated at 50° C. for 10 min. If the shell of microcapsule is successfully cross-linked, the capsules should become thermostable and remain intact, and if a cross-linking reaction is unsuccessful the microcapsules will dissolve or will end up defective. Results shown in FIG. 18C show that performing a cross-linking reaction with 0.3 U/mL of mTG for 30 min at 23° C. was sufficient to obtain microcapsules. Results shown in FIG. 18D also confirm that performing a cross-linking reaction with 0.3 U/mL of mTG for 1h at 4° C. was sufficient to obtain microcapsules. Therefore, the production of the disclosed microcapsules can be obtained in variety of ways such as using chemical agents (e.g., LAP, TEMED, APS, etc), using cross-linking agents (e.g., genipin), and using enzymes (e.g., mTG).

### Example 6: a Variety of Polyhydroxy Compounds May Constitute the Microcapsule's Core

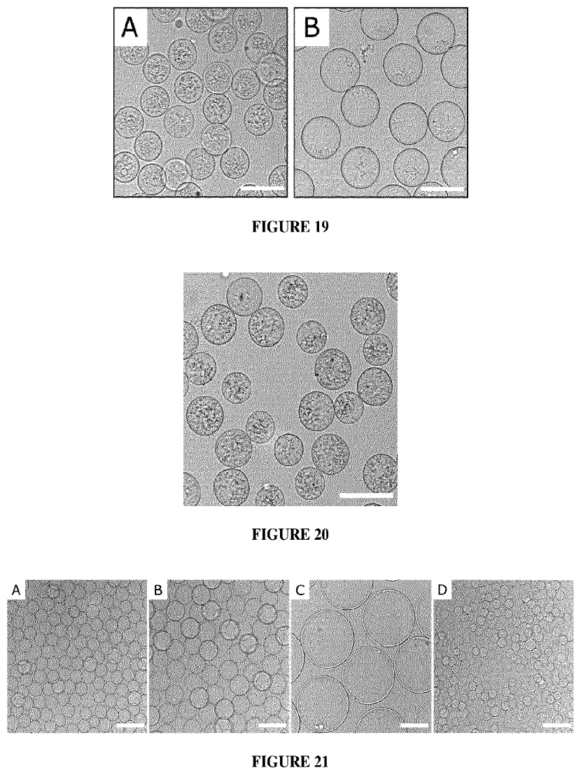

It should be understood that the core of capsules is not constrained to the use of dextran. Other polyhydroxy compounds (e.g. carbohydrates, sugars, oligosaccharides, polysaccharides, synthetic polymers) may be used instead of dextran, or polyhydroxy compounds can be mixed at different ratios. For example, FIG. 19 shows capsules where the dextran phase was entirely replaced with hydroxyethyl-cellulose (FIG. 19A) or Ficoll PM400 (FIG. 19B). In one non-limiting example the dextran phase with 30% (w/v) Ficoll PM400 (Sigma-Aldrich, GE17-0300-10) solution in 1×PBS buffer. In a second example, generation of capsules is achieved by replacing the dextran phase with 3% (w/v) hydroxyethyl-cellulose (Sigma-Aldrich, 09368-100G) solution in 1×PBS buffer. In both examples the aqueous phase forming the shell contained 3% (w/v) GMA in 1×PBS buffer. Also, in both examples the capsules were generated using a microfluidics chip having 40 μm deep channels and flow rates for GMA solution were 250 μl/h, for Ficoll PM400 or hydroxyethyl-cellulose—100 μl/h and for carrier oil—700 μl/h. Emulsifications were performed at room temperature and resulting droplets were collected off-chip into 1.5 ml tube. The collected droplets were incubated at 4° C. for 30 minutes to induce gelatin solidification and thereby the capsule's shell formation. The capsules were then resuspended in an ice-cold 1×PBS buffer supplemented with 0.1% Pluronic F-68 and 0.1% (w/v) LAP and photo-polymerized under exposure to 405 nm LED device for 20 seconds. The resulting capsules were inspected under the bright field microscope and are shown in FIG. 19A and FIG. 19B.

### Example 7: Replacing the Polyhydroxy Compounds Constituting the Core with Solution Comprising the Salt Solution

Having shown a production of microcapsules comprising a proteinacious shell and a core made of different types of polyhydroxy compounds, we consider that microcapsules can also be made where the polyhydroxy compound used for the core is replaced with an antichaotropic agent such as an aqueous solution containing a kosmotropic salt. To demonstrate such a possibility, we generated microcapsules where the liquid core was an aqueous solution comprising ammonium sulfate. Microcapsules were generated using a microfluidics chip having 40 μm deep channels and the flow rate for 3% (w/v) GMA solution at 175 μl/h, for 1M ammonium sulfate at 175 μl/h and for carrier oil at 700 μl/h. Emulsification was performed at room temperature and resulting water-in-oil droplets were collected off-chip into 1.5 ml tube. The collected droplets were then incubated at 4° C. for 30 minutes to induce gelatin solidification and thereby the intermediate microcapsule formation. Continuing procedures on ice, the microcapsules were recovered from the emulsion (as described above) and resuspended in an ice-cold 1×PBS buffer supplemented with 0.1% (w/v) Pluronic F-68 and 0.1% (w/v) LAP. The microcapsules were cross-linked by photo-polymerization under exposure to 405 nm LED device (Droplet Genomics, DG-BR-405) for 20 seconds. The resulting capsules were inspected under the bright field microscope and are shown in FIG. 20.

Although in the examples above preferably only the shell of capsules is solidified while keeping the core of the capsules in a liquid form, it should be understood for the experienced in the field that capsule core can be also polymerized into a desirable strength hydrogel mesh by adding a cross-linking agent soluble in the core phase, or soluble in both core/shell forming phases. In such case the microcapsules will contain the solidified shell and solidified core of different stiffness. One such example has been revealed by our previous invention (US20200400538A1), where the cross-linking agent, PEGDA, distributed in both phases yet with a higher quantity partitioning at the shell phase.

### Example 8: Generation of Microcapsules Having Different Size

When using a microfluidics device having microchannels ranging from 20 to 80 μm deep, the size of microcapsules could be tuned between 35 and 200 μm by simply changing the flow rates of the system, and without significantly affecting their concentricity. For example, using a microfluidics device 20 μm deep, the microcapsules having the size in the range of 35 to 45 μm were generated by tuning the flow rate of the injected fluids (FIG. 21A). Accordingly, using a microfluidics device 40 μm deep the size of capsules could be tuned between 60 and 85 μm (FIG. 21B). Using a microfluidics device 80 μm deep the size of capsules could be tuned in the range of 150 to 200 μm in diameter (FIG. 21C). These results show that tuning the flow rates of the system and/or changing the cross-section of the channels it is possible to generate microcapsules of a desirable diameter. It should be possible to generate even smaller or larger capsules by employing a microfluidic system having a smaller or larger cross-section channel, or having a smaller/larger size nozzle, respectively. Alternatively, the smaller size capsules can be generated by employing a geometrically mediated breakup of droplets [87]. For example, capsules shown in FIG. 21D were generated using a geometrically mediated breakup, which resulted in 24 μm diameter capsules.

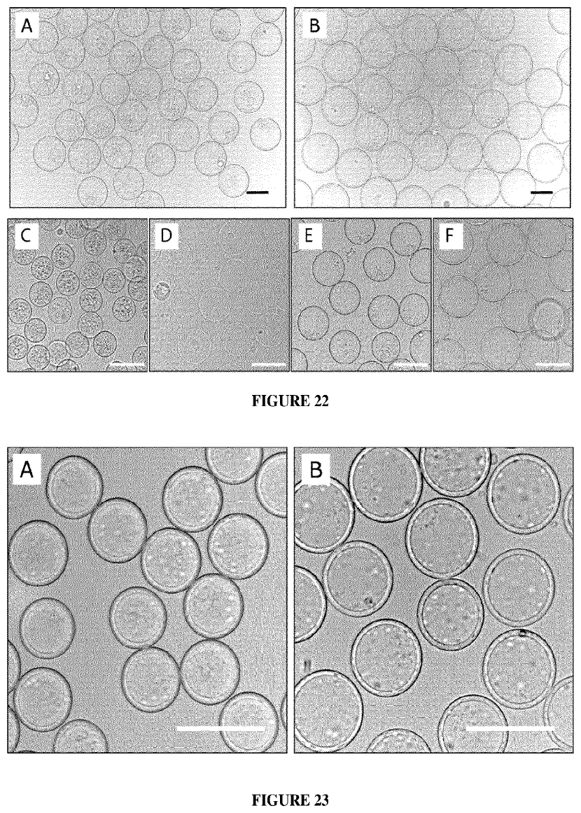

As explained below capsule size can be controlled not only by the flow rates or the cross-section of the microfluidic channels, but also by the temperature. To prove the effect of temperature on capsules size the GMA/dextran capsules were generated using a microfluidics chip having 80 μm deep channels. The flow rates for GMA solution were 200 μl/h, for dextran—50 μl/h and for carrier oil—500 μl/h. Emulsion was collected off-chip into 1.5 ml tube at room temperature and placed at 4° C. for 30 minutes to solidify the shell. Next, emulsion was divided into two fractions and processed separately at different temperatures:
The capsules in the first fraction were recovered from the emulsion, resuspended in an ice-cold 1×PBS buffer supplemented with 0.1% (w/v) Pluronic F-68 and 0.1% (w/v) LAP, and photo-polymerized under 405 nm wavelength for 20 seconds. The resulting capsules are shown in FIG. 22A.
The capsules in the second fraction were recovered from the emulsion, resuspended in an ice-cold 1×PBS buffer supplemented with 0.1% (w/v) Pluronic F-68 and 0.1% (w/v) LAP, and incubated at room temperature (˜22° C.) for 15 min to allow capsule swelling to occur. Following incubation, the capsules were photo-polymerized under 405 nm wavelength for 20 seconds. The resulting capsules are shown in FIG. 22B.

In yet another example the effect of temperature on capsule size is revealed by producing capsules with a core composed of polyhydroxy compounds other than dextran. The capsules of choice were produced from GMA/hydroxyethyl-cellulose or GMA/Ficoll PM400 blend. In one example, the first aqueous phase contained 30% (w/v) Ficoll PM400 solution in 1×PBS buffer and the second aqueous phase contained 3% (w/v) GMA in 1×PBS. In another example, the first aqueous phase contained 3% (w/v) hydroxyethyl-cellulose solution in 1×PBS and the second aqueous phase contained 3% (w/v) GMA in 1×PBS buffer. In both examples the capsules were generated using a microfluidics chip having 40 μm deep channels and flow rates for GMA phase at 250 μl/h, for Ficoll PM400 or hydroxyethyl-cellulose—100 μl/h and for carrier oil—700 μl/h. Emulsifications were performed at room temperature. The collected droplets were incubated at 4° C. for 30 minutes to induce solidification of the shell and thereby the intermediate microcapsule formation. Next, emulsion was divided into two fractions. The emulsion in the first fraction was further processed on ice, whereas the emulsion in the second fraction was processed at room temperature following incubation on ice.
The capsules in the first fraction were dispersed in an ice-cold 1×PBS buffer supplemented with 0.1% (w/v) Pluronic F-68 and 0.1% LAP, and photo-polymerized under 405 nm light for 20 seconds. The resulting polymerized capsules were inspected under the bright field microscope and are shown in FIG. 22C and FIG. 22E.
The capsules in the second fraction were resuspended in an ice-cold 1×PBS buffer supplemented with 0.1% (w/v) Pluronic F-68 and 0.1% LAP, and transferred to room temperature (˜22° C.) for 15 min to induce the capsule swelling and expansion. Following the incubation at room temperature the capsules were photo-polymerized under 405 nm light for 20 seconds. The resulting capsules are shown in FIG. 22D and FIG. 22F.

In summary, the results presented in FIG. 22 prove that capsule size can be controlled by temperature. When microcapsules are composed of polyampholyte such as gelatin the preincubation of capsules at temperature higher than 4° C., and preferably at 22° C., prior to the cross-linking step, leads to larger size capsules.

### Example 9: Controlling the Shell Thickness of the Microcapsules

In addition to achieving the precise control over the capsule size (see Example 8), the shell thickness could be also tuned by adjusting the flow rates of the system, the concentration of the shell forming polymer. In one example, capsules generated using a microfluidics device 20 μm deep had a shell 2 μm thick (FIG. 21A). In another example, capsules generated using a microfluidics device 40 μm deep had a shell 3 μm thick (FIG. 21B). In yet another example, capsules generated using a microfluidics device 80 μm deep had a shell 5 μm thick (FIG. 21C).

The shell thickness of the capsules could be also tuned by adjusting the concentration of the shell forming polymer. FIG. 23A shows 68 μm size capsules having 6.5 μm shell and 55 μm core. Such capsules were generated by emulsifying 5% GMA with 15% dextran solutions followed by temperature-induced gelation and photo-induced cross-linking of the shell. As expected, pre-incubation at room temperature (˜22° C.) for 15 min before photo-polymerization increased the size of capsules to −82 μm diameter (70 μm core and 6 μm shell) as shown in FIG. 23B.

Altogether the examples presented above show successful microcapsule generation using a number of different components, and cross-linked using different agents such as temperature, light, chemical agents or biological reagents (e.g., enzyme). The microcapsule size and shell thickness can be tuned by changing cross-section dimensions of the channels, geometry of microfluidic channels, the volumetric ratio of fluids during emulsification step, the concentration of the ingredients in the liquid phases, the share force generated by carrier oil, or by changing the temperature at which capsules are generated/or processed.

### Example 10: Microcapsules for Efficient Cell Isolation and Retention

To evaluate cell encapsulation and retention efficiency we used K-562 (ATCC, CCL-243) and HEK293 (ATCC, CRL-1573) cell lines. First, ˜200,000 cells were re-suspended in 100 μL of dextran solution (MW 500k) (Sigma-Aldrich, 31392-10G). The microcapsules were generated using a microfluidics chip 40 μm height and having a nozzle 40 μm wide. The flow-rates used were, 250 μl/hr for 3% (w/v) gelatin methacrylate (Sigma-Aldrich, 900496-1G) dissolved in 1×PBS buffer; 100 μl/h for 15% (w/v) dextran solution carrying cells and 700 μl/hr for carrier oil. Encapsulations were performed at room temperature (˜22° C.) and resulting water-in-oil droplets were collected off-chip into laboratory tube. After encapsulation, the emulsion was transferred to 4° C. and incubated for 30-60 minutes to complete the temperature-induced gelation of the shell. The microcapsules were recovered from the emulsion using commercial emulsion breaker (Droplet Genomics, DG-EB-1) and resuspended in ice-cold 1×PBS, supplemented with 0.1% (w/v) Pluronic F-68 (Gibco, 24040032). The intermediate-microcapsule suspension was transferred into a new laboratory tube, supplemented with 0.1% (w/v) lithium phenyl-2,4,6-trimethylbenzoylphosphinate (Sigma-Aldrich, 900889-1G) and photo-polymerized under exposure to 405 nm LED device (Droplet Genomics, DG-BR-405) for 20 seconds. After photopolymerization, the microcapsules were rinsed twice in 1×PBS buffer containing 0.1% Pluronic F-68, and then resuspended in 1 ml of cell culture media [1×IMDM (Gibco, 12440053) supplemented with 10% FBS (Gibco, 11573397) and 1× Penicillin-Streptomycin (Gibco, 15070063)].

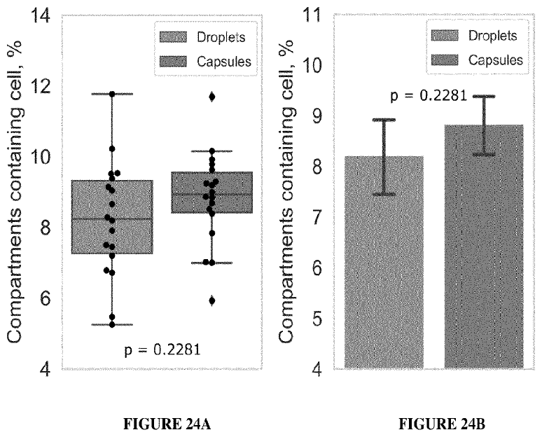

We quantified cell retention microscopically immediately after encapsulation, and then after microcapsule formation (cross-linking), and confirmed that the capsules efficiently retained encapsulated cells (FIGS. 24A and B). No significant difference was detected (two-tailored t-test (n=18) t=1.22, p=0.2281) confirming that compartmentalized cells were efficiently retained through all the steps of capsule generation. These results are in sharp contrast to PEGDA/Dextran composition, where majority of cells either moved to the shell phase, or to the shell/core interface (FIG. 9), or escaped compartmentalization (FIG. 10 and FIG. 11).

### Example 11: Comparison of Cell Encapsulation and Long-Term Culture in Microcapsules and in Hydrogel Beads

To evaluate microcapsule suitability for long term cell culture as well as to investigate cell encapsulation and retention efficiency and formation of 3D cell structures (e.g. spheroids) originating from a single mammalian cell, we first performed a study using K-562 cells (ATCC, CCL243). In addition, the 3D cell culture inside the hydrogel beads composed of either ultra-low melting point agarose (Zehao Chemical, 9012-36-6) or gelatin methacrylate (Sigma-Aldrich, 900629-1G) was evaluated and compared to 3D cell culture inside the microcapsules of this invention. In all three systems the K-562 cells were encapsulated in water-in-oil droplets at a limiting dilution such that each compartment, on average, would contain no more than one cell. Cell encapsulation using droplet microfluidics is a well described process governed by Poisson statistics [82].

Microcapsules with Proteinaceous Shell: The microcapsules having cells were generated as described above. First, K-562 cells were re-suspended in 100 μL of dextran solution (MW 500k) (Sigma-Aldrich, 31392-10G) at the final concentration ˜2 mln/ml. The microcapsules were generated using a microfluidics chip 40 μm height and having a nozzle 40 μm wide. The flow-rates used were, 250 μl/hr for 3% (w/v) gelatin methacrylate (Sigma-Aldrich, 900496-1G) dissolved in 1×PBS buffer; 100 μl/h for 15% (w/v) dextran solution carrying cells and 700 μl/hr for the carrier oil. Encapsulations were performed at room temperature (˜22° C.) and resulting water-in-oil droplets were collected off-chip into laboratory tube, prefilled with 200 μl of light mineral oil (Sigma-Aldrich, M5904-500ML). After encapsulation, the emulsion was transferred to 4° C. and incubated for 30-60 minutes to induce temperature-induced physical gelation of gelatin. The microcapsules were recovered from the emulsion using commercial emulsion breaker (Droplet Genomics, DG-EB-1) and resuspended in ice-cold 1×PBS, supplemented with 0.1% Pluronic F-68 (Gibco, 24040032). The microcapsule suspension was transferred into a new laboratory tube, supplemented with 0.1% (w/v) lithium phenyl-2,4,6-trimethylbenzoylphosphinate (Sigma-Aldrich, 900889-1G) and photo-polymerized under exposure to 405 nm LED device (Droplet Genomics, DG-BR-405) for 20 seconds. After photopolymerization, the microcapsules were rinsed twice in 1×PBS buffer containing 0.1% Pluronic F-68, and then resuspended in 1 ml of cell culture media [1×IMDM (Gibco, 12440053) supplemented with 10% FBS (Gibco, 11573397) and 1× Penicillin-Streptomycin (Gibco, 15070063)]. The microcapsule suspension was then divided into five 35 mm size Petri dishes (Thermo Scientific, 130180); where each Petri dish contained ˜200 μL of microcapsule suspension and 1.8 ml cell culture media. The Petri dishes with microcapsules were transferred to a cell incubator at 37° C., 95% air, 5% CO₂.

Polyampholyte (gelatin-based) Hydrogel Beads: To encapsulate cells in proteinaceous hydrogel beads the K-562 cells were first re-suspended in 100 μl of 1×PBS containing 20% (v/v) OptiPrep (Sigma-Aldrich, D1556-250ML) and co-encapsulated in water-in-oil droplets along with 4% (w/v) gelatin methacrylate solution. The microfluidics chip used had microchannels 40 μm height and a nozzle 40 μm wide. The flow-rates used were, 75 μl/hr for cell suspension, 250 μl/hr for 4% (w/v) gelatin methacrylate (Sigma-Aldrich, 900496-1G) dissolved in 1×PBS buffer, and 300 μl/hr for the carrier oil. Cell encapsulation was performed at room temperature for −20 minutes, emulsion collected in 1.5 mL tube, prefilled with 200 μl of light mineral oil (Sigma-Aldrich, M5904-500ML). The following steps, including hydrogel bead polymerization, recovery and culture initiation, were the same as described above for the microcapsules with proteinaceous shell. The hydrogel beads were recovered from the emulsion using commercial emulsion breaker (Droplet Genomics, DG-EB-1) and resuspended in ice-cold 1×PBS, supplemented with 0.1% Pluronic F-68 (Gibco, 24040032). The hydrogel bead suspension was transferred into a new laboratory tube, supplemented with 0.1% (w/v) lithium phenyl-2,4,6-trimethylbenzoylphosphinate (Sigma-Aldrich, 900889-1G) and photo-polymerized under exposure to 405 nm LED device (Droplet Genomics, DG-BR-405) for 20 seconds. After photopolymerization, the hydrogel beads were rinsed twice in 1×PBS buffer containing 0.1% Pluronic F-68, and then resuspended in 1 ml of cell culture media [1×IMDM, 10% FBS and 1× Penicillin-Streptomycin]. Next, five equal parts of hydrogel bead suspension (200 μl each) was transferred to five 35 mm size Petri dishes (Thermo Scientific, 130180) where each contained 1.8 ml cell culture media. The Petri dishes with microcapsules were transferred to a cell incubator at 37° C., 95% air, 5% CO₂ for further incubation.

Polyelectrolyte (agarose-based) Hydrogel Beads: To perform cell encapsulation in agarose beads, the K-562 cells were re-suspended in 2.0% (w/v) ultra-low melting point agarose (Zehao Chemical, 9012-36-6) dissolved in 1×PBS and loaded into microfluidics chip to generate water-in-oil droplets. Microfluidics chip was 40 μm height and having a nozzle 40 μm wide. The flow-rates used were 325 μl/h for cell suspension in agarose and 700 μl/hr for the carrier oil. Encapsulation was performed at room temperature for ˜20 minutes, and emulsion collected in 1.5 ml tube, prefilled with 200 μl of light mineral oil (Sigma-Aldrich, M5904-500ML). After cell encapsulation, water-in-oil droplets were immediately transferred at 4° C. for ˜30 minutes incubation to induce agarose gelation. Agarose beads were recovered from the emulsion using commercial emulsion breaker (Droplet Genomics, DG-EB-1) and 1×PBS supplemented with 0.1% Pluronic F-68. Hydrogel beads were rinsed twice in 1×PBS containing 0.1% Pluronic F-68, and then resuspended in 1 ml of cell culture media [1×IMDM, 10% FBS and 1× Penicillin-Streptomycin]. Next, five equal parts of agarose bead suspension (200 μl each) was transferred to five 35 mm size Petri dishes (Thermo Scientific, 130180) where each dish contained 1.8 ml cell culture media. The Petri dishes with microcapsules were transferred to a cell incubator at 37° C., 95% air, 5% CO₂ for further incubation.

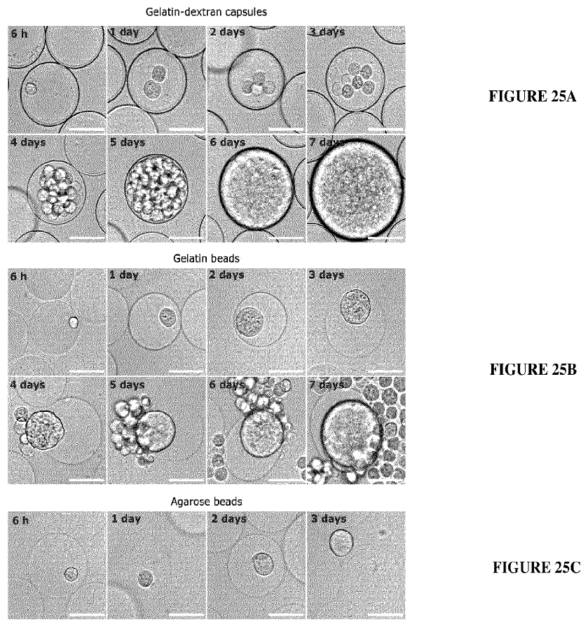

Results show in FIG. 25A-C indicate that encapsulated cells expanded into 3D cell structures in all three cases. However, only the cells encapsulated in microcapsules were retained during long-term culture (FIG. 25A). Cells cultured in polyampholyte beads based on gelatin biomaterial escaped the compartmentalization after 4-7 days of culture (FIG. 25B). Cells cultured in polyelectrolyte beads based on agarose biomaterial escaped the compartmentalization after 3 days of culture (FIG. 25C). These results prove that the microcapsules of this disclosure support long-term culture of encapsulated cells in 3D environment. The results presented in FIG. 25A also reveal that as cells continue to divide and expand within the microcapsule, it does not burst, thus indicating that the cross-linked shell is elastic and may be deformed without breaking/bursting while allowing the cell culture to reach confluency. The results presented in FIG. 25A also confirm that microcapsule of this invention supports 3D cell culture and formation of 3D cell structures.

To ensure that encapsulated cells are not compromised by biomaterials used to generate microcapsules and beads described above, a cell viability test was conducted. After encapsulations and 6 hours of cell culture, cell viability was evaluated using two DNA fluorescent dyes: SYTO 9 (Invitrogen, S34854), which stains nuclei acid in both live and dead cells, and ethidium homodimer-1 (Invitrogen, E1169), which stains nuclei acids in dead cells with compromised membranes. In Petri dishes with 2 ml of culture 1 μl of SYTO 9 and 4 μl of ethidium homodimer-1 (EthD-1) was added and incubated for 30 minutes in a cell culture incubator. Next, microcapsules were collected, rinsed twice in 1×PBS containing 0.1% Pluronic F-68 and viability of cells evaluated under the fluorescence microscope (Nikon Eclipse). The cell viability in microcapsules, gelatin-based beads and agarose-based beads was similar: 95.2%, 98.2% and 91.5%, respectively.

### Example 12: Microcapsule's Shell Provides a Support for which the Cells Attach

To demonstrated that microcapsule shell can act as a substrate for cells to attach to we reveal a proof-of-concept experiment using two different adherent cell lines as a model system MDA-MB-231 (ATCC, HTB-26) and A549 (ATCC, CRM-CCL-185). The said cells were encapsulated and cultured inside the microcapsules for a period over 10 days.

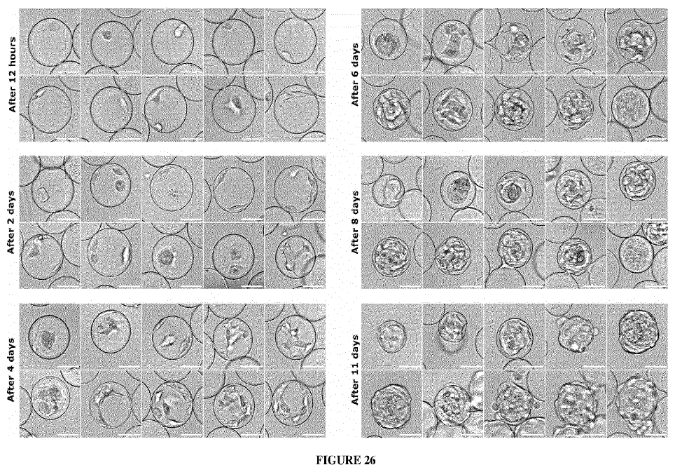

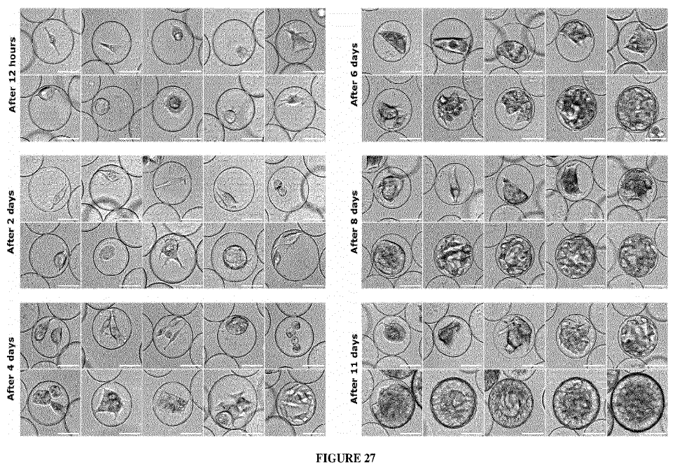

Following the microcapsule generation procedure described in Example 11, the MDA-MB-231 and A549 cells were separately encapsulated at a limiting dilution such that each microcapsule, on average, would contain no more than one cell. Briefly, microcapsules were generated using a microfluidics chip 40 μm height and having a nozzle 40 μm wide. Typical flow-rates used were: 3% gelatin methacrylate solution—250 μl/h, 15% dextran solution with cells—100 μl/h and the carrier oil—700 μl/h. Cell encapsulations were performed at room temperature (21-22° C.) for 20-25 minutes and resulting water-in-oil droplets were ere collected in the form of an emulsion in 1.5 ml tube. Following cell encapsulation, the emulsions were transferred to 4° C. for 30-60 minutes for gelatin solidification to occur. Continuing procedures on ice, the intermediate-microcapsules were recovered from the emulsion using commercial emulsion breaker (Droplet Genomics, DG-EB-1) and resuspended in 1×PBS, supplemented with 0.1% Pluronic F-68. Capsule suspension was transferred by pipetting into a new 1.5 ml tube, supplemented with 0.1% (w/v) LAP and photo-polymerized under exposure to 405 nm LED device (Droplet Genomics, DG-BR-405) for 20 seconds. After cross-linking step, the reusing microcapsules (approx. 250-300 μl in volume) were rinsed twice in 1×PBS supplemented with 0.1% Pluronic F-68, and then resuspended in 1 ml of 1×DMEM supplemented with 10% FBS and 1× Penicillin-Streptomycin. Next, 1 ml of microcapsule suspension was divided in five 35 mm Petri dishes (Thermo Scientific, 130180): 200 μL of microcapsule suspension transferred in 1.8 ml culture medium. Petri dishes with capsules were transferred to a cell incubator and incubated at 37° C., 95% air, 5% CO₂ environment for over 10 days. At selected time points the microcapsules in the Petri dishes were imaged under bright field microscope to evaluate cell growth and adhesion to the inner surface of the microcapsule. The results presented in FIGS. 26 and 27 show that encapsulated cells successfully attached to the inner surface (core/shell interface) of the microcapsule rather quickly. Human breast adenocarcinoma cells (MDA-MB-231) appear to attach to the inner surface of the microcapsules during the 12 hours of cell culture and subsequently grow into complex 3D cell structures over the few days of culture (FIG. 26). Similarly, human alveolar basal epithelial cells (A549) appear to attach to the inner surface of the microcapsules during the 12 hours of cell culture and subsequently expand into complex 3D cell structures (FIG. 27).

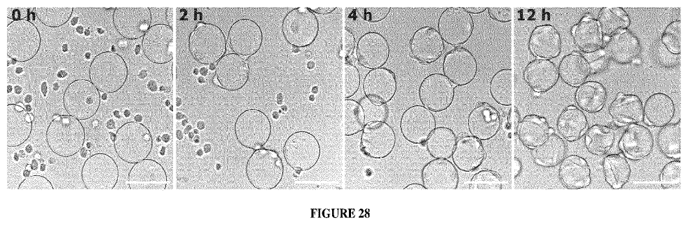

To demonstrate that cells can also attach to the outside surface of the microcapsule we performed a proof-of-concept experiment where the microcapsules (produced from gelatin methacrylate and dextran) were mixed with MDA-MB-231 cells and incubated in 1×DMEM medium supplemented with 10% FBS and 1× Penicillin-Streptomycin for 12 hours. The results presented in FIG. 28 reveal that cells started to adhere to the outer surface of the microcapsules during 2 hours of incubation at 37° C./5% CO₂ and nearly all cells adhered to the microcapsules after 12 hours of incubation. Therefore, the microcapsules revealed in this could be find many useful applications for cell co-culture, for example where one species is located inside the capsule, and another one is located outside.

### Example 13: Microcapsule Use for 3D Cell Culture and Cell-Cell Communication and/or Interaction

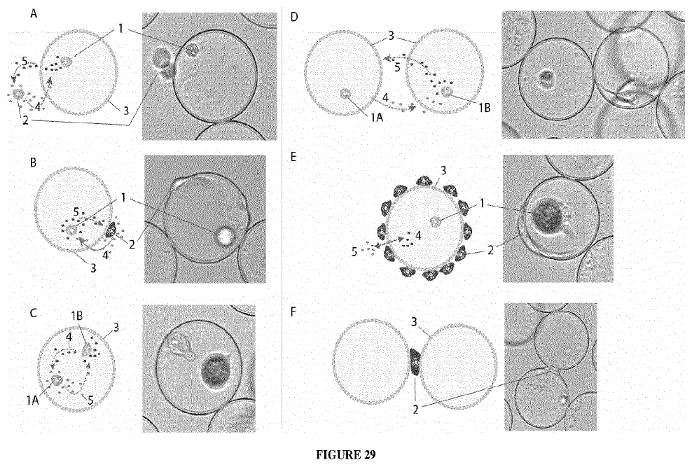

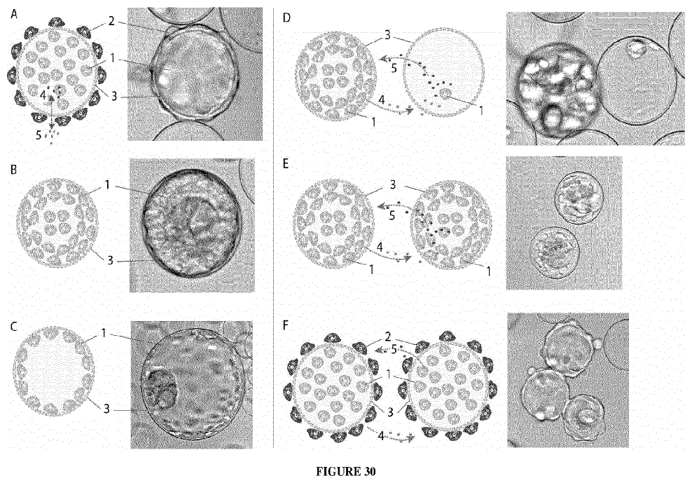

From the example presented above it will be clear for a person experienced in the art that the microcapsules of the present disclosure are applicable for a variety of experiments involving 3D cell culture. For example, the microcapsule of this disclosure may be used as a biocompatible compartment for encapsulating a cell, or more than one cell, whereas the said cell(s) may be cultured and allowed to form 3D cell assemblies (structures). The microcapsule may provide a 3D microenvironment and enable in vitro or in vivo culture of 3D cell culture. The encapsulated cells may form 3D cell structures (assemblies) such as spheroids, organoids, tumoroids, tissues, assemblies, clumps and other cell clusters. The microcapsule carrying one, two, three, four, five or more than five cells can be cultured in suitable in vitro or in vivo conditions to enable formation of spheroids, organoids, tumoroids, tissues and other types of 3D cell structures. Some of the selected but non-limiting examples shown in FIG. 29A-F and FIG. 30A-F reveal the microcapsule's use for conducting cell-based assays.

FIG. 29A exemplifies the cell assay where the cell inside the microcapsule is incubated in the suspension having cell outside the microcapsule. The cells may or may not communicate biochemically via soluble factors.

FIG. 29B exemplifies the cell assay where the cell outside the microcapsule attaches to the microcapsule carrying the cell of interest. The cells may or may not communicate biochemically via soluble factors through the microcapsule's shell.

FIG. 29C exemplifies the cell-based assay where the two cells reside in the same microcapsule and may or may not communicate biochemically via soluble factors and physically via cell-cell interactions.

FIG. 29D exemplifies the cell-based assay where the cells reside in different microcapsules and may or may not communicate biochemically via secreted factors.

FIG. 29E exemplifies the cell-based assay where the cells attach to the outer surface of the microcapsule, which comprises a cell (or several cells). The cells inside the microcapsule and the cells outside the microcapsule may or may not communicate biochemically via secreted factors.

FIG. 29F exemplifies the case when the cell attaches to the outer surface of the microcapsule and brings two microcapsules in proximity. The said cell may act as a bridge that can bring in proximity, two, three or more microcapsules with or without cells.

FIG. 30A shows an example of the cell-based assay where the cells attach to the outer surface of the microcapsule and form a layer (e.g., monolayer, multilayer), whereas the cells inside the microcapsule form a 3D cell assembly. The cells outside the microcapsule may or may not communicate biochemically via secreted factors with the cells inside the microcapsule.

FIG. 30B exemplifies microcapsule use for the formation of a 3D cell assembly comprising multiple layers of cells. The cells may or may not communicate biochemically via secreted factors and/or physically via cell-cell interactions.

FIG. 30C exemplifies microcapsule use for the formation of a 3D cell assembly comprising a layer of cells attached to the inner surface of the microcapsule.

FIG. 30D exemplifies a cell-based assay where the 3D cell assembly (e.g., spheroid) in one microcapsule is suspended in a suspension having microcapsules comprising a cell (or several cells), wherein the cells inside the microcapsules may or may not communicate biochemically via secreted factors.

FIG. 30E exemplifies the cell-based assay where the 3D cell assembly (e.g., spheroid) of one cell type present in one microcapsule is suspended in the suspension having microcapsules comprising the 3D cell assembly of another cell type, wherein the cells inside the microcapsules may or may not communicate biochemically via secreted factors.

FIG. 30F exemplifies the cell-based assay where the 3D cell assembly comprising cells attached to the outer surface of the microcapsule and cells residing inside the same microcapsule are assayed in a mix comprising another type of cells forming a 3D cell assembly comprising cells attached to the outer surface of the microcapsule and cells residing inside the same microcapsule. The cells inside the microcapsules may or may not communicate biochemically via secreted factors and/or physically via cell-cell interactions.

Altogether, these non-limiting examples show that the microcapsule shell can act as a substrate for cells to attach to, irrespectively if the cells are inside or outside the microcapsule. These examples also show that the microcapsules can serve as a bioreactor for harvesting and culturing cells, and that complex interactions between the cells can be studied in a spatially controlled manner.

### Example 14: Cell Recovery from Microcapsules by Dissolving Proteinaceous Shell

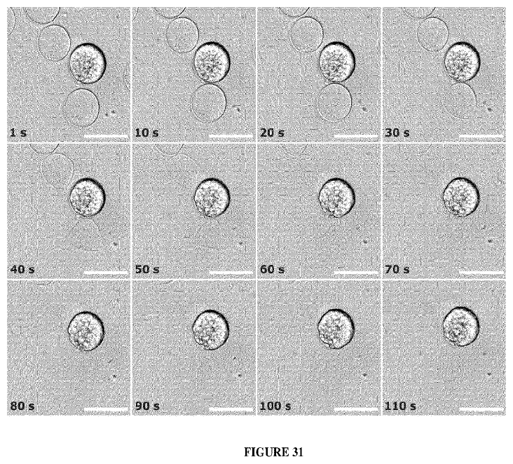

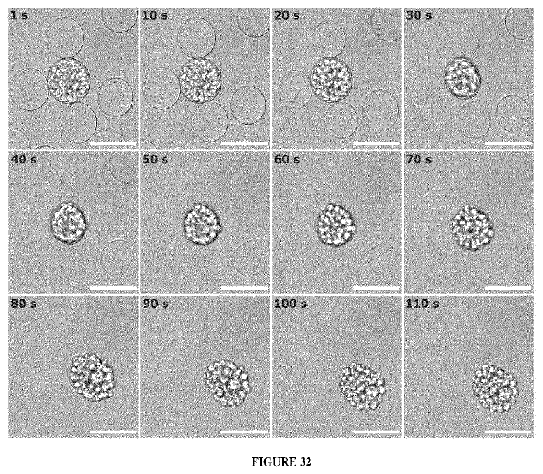

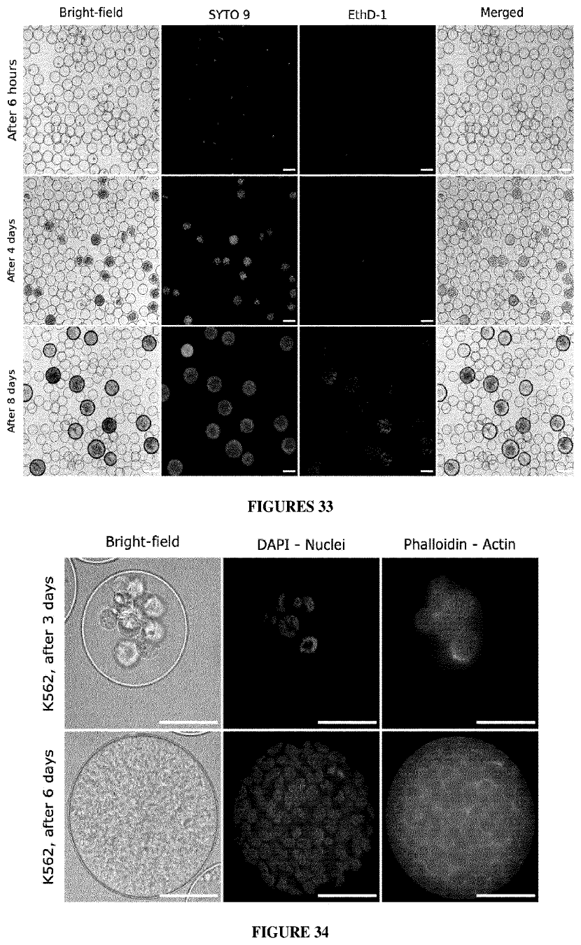

For some biological assays, cell encapsulation and spheroid formation are only the first steps in the workflow. For example, spheroids of interest could be selected and cells forming the spheroids isolated for the downstream analysis. Due to these reasons, viable cell recovery from the capsules is an important characteristic for a fully functional and applicable culture system. Since the microcapsule's shell comprises peptide bonds, the enzymes such as proteases (e.g., collagenase, trypsin etc.) can be employed to digest (disintegrate, decompose) the shell and release the encapsulated cells. To illustrate such a possibility the microcapsules comprising K562 cells or comprising HEK293 cells were generated as described in the Example 10. The microcapsules were incubated for a few days in a cell growth medium [1×IMDM, 10% FBS and 1× Penicillin-Streptomycin] in a cell incubator at 37° C./5% CO₂ to produce encapsulated cells reaching the confluency. Next, the microcapsules having 3D cell assemblies were rinsed twice in 1×PBS buffer, containing 0.1% (v/v) Pluronic F-68 and treated with 0.5 mg/ml collagenase A in the presence of 0.5 mM CaCl₂. The microcapsule integrity and encapsulated cell release was followed over time. Results shown in FIG. 31 and FIG. 32 confirm that microcapsules can be decomposed upon treatment with protease and entire 3D cell assembly released into the surrounding medium.

### Example 15: Evaluation of Cell Viability During Cell Culture and Harvesting Using Microcapsules

To evaluate the encapsulated cell viability during cell culture and harvesting inside the microcapsules of disclosed invention the K562 cells were used as a model system. The microcapsules comprising K562 cells were generated as described in the Example 10. The microcapsules were incubated in a cell growth medium [1×IMDM, 10% FBS and 1× Penicillin-Streptomycin] in a cell incubator at 37° C./5% CO₂ and at selected time points the viability of cells was evaluated using fluorescent dyes (SYTO 9 and Ethidium homodimer-1). 2 ml of culture medium having dispersed microcapsules were mixed with 1 μl of SYTO 9 and 4 μl of ethidium homodimer-1 (EthD-1) and incubated for 30 minutes in a cell culture incubator. Next, microcapsules were collected, rinsed twice in 1×PBS containing 0.1% Pluronic F-68 and viability of cells evaluated under the fluorescence microscope. Results presented in FIG. 33 show that cells remain highly viable for a few days of culture inside the microcapsules. Few dead cells appear on day 8 due to cell confluency, stress, or other factors.

### Example 16: Microcapsule-Derived 3D Cell Assembly Fixation Followed by Staining

One of the most common methods for analysis of 3D cell assemblies such as organoids or spheroids relies on fluorescence readout of paraformaldehyde (PFA) fixed samples. To exemplify the use of microcapsules for this type of analysis, we performed a proof-of-concept experiment involving PFA fixation, permeabilization and staining of 3D cell assembly. The microcapsules comprising K562 cells were generated as described in the Example 10. The microcapsules were incubated in a cell growth medium and at selected time points the microcapsules were washed in 1×PBS and approximately 50 μl of closely packaged microcapsules were immersed in 1 ml of 4% (w/w) PFA dissolved in 1×PBS. After fixation at room temperature for 15 minutes, the microcapsules were rinsed three-times in 1×PBS. Next, the PFA-treated microcapsules were washed in 1 ml of 1×PBS containing 0.1% (v/v) Triton X-100 and incubated at room temperature for 15 minutes. Next, the microcapsules were washed three-times in 1×PBS, containing 1 ml of 1% (w/v) BSA.

The PFA-fixed cells were stained for actin and nuclei, using phalloidin and DAPI dyes, respectively. Specifically, for staining actin, microcapsule suspension was treated with ActinGreen 488 ReadyProbes Reagent (Invitrogen, R37110) and incubated at room temperature in the dark for 30 minutes. Then, microcapsule suspension was supplemented with DAPI to the final concentration of 300 nM and incubated for additional 30 minutes at room temperature in the dark. Finally, capsules were washed three times in 1 ml of 1×PBS and analyzed under fluorescence microscope, using FITC and DAPI filers. The results presented in FIG. 34 show that microcapsules withstand PFA treatment and enables fluorescence readout of encapsulated cells and complex 3D cell assemblies and structures.

### Example 17: Cell Culture in Dextranase Treated Capsules

The polyhydroxy compound constituting the microcapsule's core such as dextran (MW 500K) increases the viscosity the microcapsule's core. Although no adverse effect on encapsulated cells have been detected, yet it is possible that some cells may respond differently to an increased viscosity in their environment. To reduce the viscosity of the microcapsule's core we exemplify the depletion of polyhydroxy compound by applying a mild enzymatic treatment using hydrolase (e.g., dextranase).

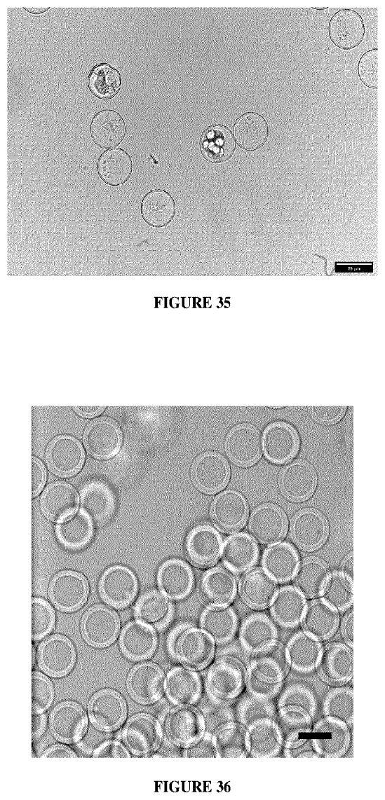

The microcapsules comprising Hela cells were generated as described in the Example 10. The microcapsules were suspended in 1 ml of 1×PBS containing 0.1% Pluronic F-68, and then supplemented with 20 μl of dextranase (Sigma-Aldrich, D0443-50ML) and incubated at room temperature (21-22° C.) for 5 minutes. After dextran depletion, the microcapsules were rinsed twice in 1×PBS containing 0.1% Pluronic F-68, and then resuspended in 1 ml of 1×IMDM containing 10% FBS and 1× Penicillin-Streptomycin. Microcapsules were resuspended in 35 mm Petri dish supplemented with 2 ml of cell culture medium and incubated in a cell incubator at 37° C., 95% air, 5% CO₂ to initiate cell growth. The FIG. 35 shows HeLa cell expansion in microcapsules after 5 days of cell culture. The cells formed 3D structures, and because of the reduced viscosity of the core some deformation of microcapsules could be observed as cell adhered to the shell.

### Example 18: Microcapsule's Shell May be Formed from a Composite Mixture of Polyampholytes

The above-described examples rely on microcapsule generation procedure, where solutions comprising shell and core components were injected separately and were mixed only inside the droplet. In the example below, we demonstrate an alternative microcapsule generation procedure, where the components for forming the microcapsule's shell and core are first combined in a tube, phase separated and only then loaded into water-in-oil droplets. Furthermore, we showed that microcapsule's shell can be formed from a composite mixture comprising modified-gelatin (having acrylate modifications) and non-modified gelatin (lacking acrylate modifications).

To prepare biphasic system, the materials listed in Table 2 were combine and centrifuged using a warm centrifuge (37-40° C.) to phase separate dextran rich phase (bottom layer) and gelatin rich phase (top layer). The gelatin-rich and dextran-rich phases were separated into separate tubes and then loaded into a microfluidics device.

TABLE 2
Composition of materials used for generation
of microcapsules with composite shell

| | | |
|---|---|---|
| Volume | Material | Final |

| | | | |
|---|---|---|---|
| 40 | μl | 10% (w/w) gelatin methacrylate | 2% (w/v) |
| 40 | μl | 10% (w/w) gelatin from porcine skin | 2% (w/v) |
| 24 | μl | 25% (w/w) dextran (MW 500K) | 3% (w/v) |
| 96 | μl | 1X PBS, pH [7.4] | — |
| 200 | μl | Final | |

Microcapsule generation was tested using two microfluidics chips: 1) 20 μm height and having a nozzle 20 μm wide and 2) 40 μm height and having a nozzle 40 μm wide. Typical flow-rates when using 20 μm microfluidics chip were: gelatin-rich phase—50 μl/h, dextran-rich phase—20 μl/h and the carrier oil—300 μl/h. Typical flow-rates when using 40 μm microfluidics chip were: gelatin-rich phase—200 μl/h, dextran-rich phase—100 μl/h and the carrier oil—500 μl/h. Encapsulations were performed at room temperature (˜25° C.) for 20-25 minutes. Emulsions were collected in 1.5 ml tube, prefilled with 200 μl of light mineral oil (Sigma-Aldrich, M5904-500ML). Next, the emulsions were transferred at 4° C. for 30-60 minutes to solidify the shell comprising gelatin polymer. Continuing procedures on ice, microcapsules were recovered from the emulsion using commercial emulsion breaker (Droplet Genomics, DG-EB-1) and 1×PBS supplemented with 0.1% Pluronic F-68. Capsule suspension was transferred by pipetting into a new 1.5 ml tube, supplemented with 0.1% (w/v) LAP and photo-polymerized under exposure to 405 nm LED device (Droplet Genomics, DG-BR-405) for 20 seconds. After photopolymerization, the microcapsules were rinsed twice in 1×PBS containing 0.1% Pluronic F-68 and inspected under the microscope. Microcapsules approx. 40 μm in diameter were obtained using a microfluidics chip 20 μm height and having a nozzle 20 μm wide. Microcapsules approx. 55 μm in diameter were obtained using a microfluidics chip 40 μm height and having a nozzle 40 μm wide.

The results presented in FIG. 36 show microcapsules comprising a shell formed from a mixture of polyampholytes, gelatin methacrylate and porcine skin gelatin. As it could be observed the shell of the microcapsules is thicker and microcapsules tend to slightly lose the concentricity. The ratio h/R is about 0.18 (where h is shell thickness, and R microcapsule's radius) and the average concentricity, O 75%. However, in the examples shown below that did not restrict such composite microcapsule use for culturing different biological species: bacteria, yeast, and mammalian cells.

### Example 19: Bacteria and Yeast Culture in Microcapsules

Following the procedure described in Example 18, Escherichia coli MG1655 and Saccharomyces cerevisiae were separately encapsulated at a limiting dilution such that each capsule, on average, would contain no more than one cell. For bacteria encapsulation, 25 μL of fresh culture with OD value 0.5 was taken, centrifuged, and re-suspended in 80 μL of dextran-rich phase. For yeast encapsulation, 80 μL of fresh culture with OD value 0.8 was taken, centrifuged, and re-suspended in 80 μL of dextran-rich phase.

Bacteria were encapsulated in approx. 40 μm diameter capsules using a microfluidics chip 20 μm height and having a nozzle 20 μm wide. Yeast cells were encapsulated in approx. 55 μm diameter capsules using a microfluidics chip 40 μm height and having a nozzle 40 μm wide. Typical flow-rates used for bacteria encapsulation were: gelatin-rich phase ˜50 μl/h, dextran-rich phase with cells ˜20 μl/h and the carrier oil—300 μl/h. Typical flow-rates used for yeast encapsulation were: gelatin-rich phase—50 μl/h, dextran-rich phase with cells—40 μl/h and the carrier oil—300 μl/h. Encapsulations were performed at room temperature (25° C.) for 20-25 minutes. Emulsions were collected in 1.5 ml tube, prefilled with 200 μl of light mineral oil (Sigma-Aldrich, M5904-500ML).

After encapsulation, emulsions were transferred to 4° C. for 30-60 minutes to solidify the gelatin mix. Continuing procedures on ice, microcapsules were recovered from the emulsion using commercial emulsion breaker (Droplet Genomics, DG-EB-1) and resuspended in 1×PBS, supplemented with 0.1% Pluronic F-68. Microcapsule suspension was then transferred to a new 1.5 ml tube, supplemented with 0.1% (w/v) LAP and photo-polymerized under exposure to 405 nm LED device (Droplet Genomics, DG-BR-405) for 20 seconds.

After photopolymerization, microcapsules were rinsed twice in 1×PBS, containing 0.1% Pluronic F-68, and then resuspended in 1 ml of culture media: LB-Miller containing 0.1% (w/v) Pluronic F-68 for bacteria and YPD containing 0.1% (w/v) Pluronic F-68 for yeast. 1 ml of microcapsule suspension was transferred in 35 mm Petri dish (Thermo Scientific, 130180), prefilled with 1 ml of corresponding culture media. Microcapsules with bacteria were incubated at 37° C. and recorded for 5 hours at 1-hour intervals. Microcapsules with yeast were incubated at 30° C. and recorded for 15 hours at 2- to 4.5-hour intervals.

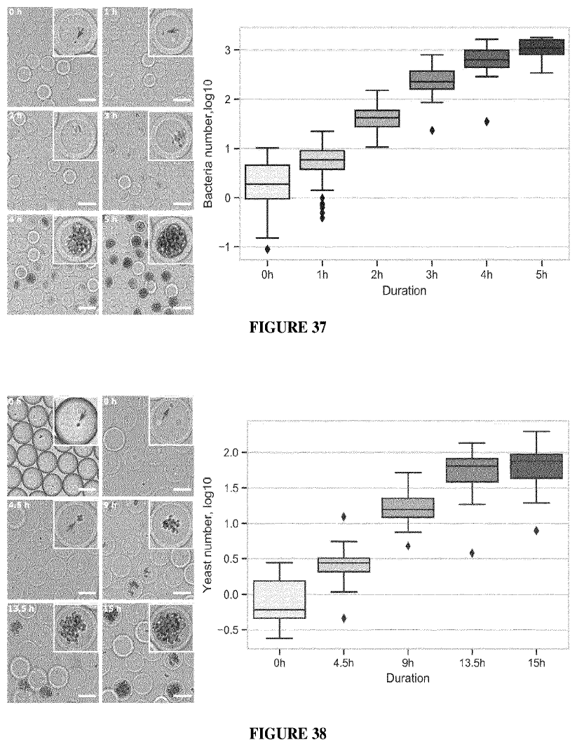

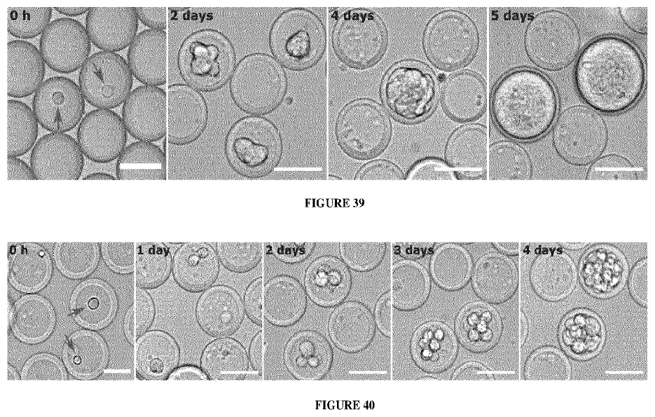

Results presented in FIGS. 37 and 38 show that both bacteria and yeast cells divided very efficiently inside the microcapsules and formed clonal microcolonies derived from single-cells.

### Example 20: Mammalian Cell Culture in Microcapsules Comprising a Composite Polyampholyte Mix

Following the procedure described in Example 18, human colon derived cells (SW620) and bone marrow derived cells (K-562) were separately encapsulated at a limiting dilution such that each microcapsule, on average, would contain no more than one cell. For that purpose, approximately 200′000 cells of each type (K-562 and SW620) were re-suspended in 100 μL of dextran-rich phase and loaded into water-in-oil droplets along with composite gelatin-enriched mix. Microcapsules were generated using a microfluidics chip 40 μm height and having a nozzle 40 μm wide. Typical flow-rates used were: gelatin-rich phase—200 μl/h, dextran-rich phase with cells—100 μl/h and for the carrier oil—500 μl/h. Encapsulations were performed at room temperature (25° C.) for 20-25 minutes. Emulsions were collected in 1.5 ml tube, prefilled with 200 μl of light mineral oil (Sigma-Aldrich, M5904-500ML).

After encapsulation, emulsions were transferred to 4° C. for 30-60 minutes to solidify the gelatin mix. Continuing procedures on ice, microcapsules were recovered from the emulsion using commercial emulsion breaker (Droplet Genomics, DG-EB-1) and resuspended in 1×PBS, supplemented with 0.1% Pluronic F-68. Microcapsule suspension was then transferred to a new 1.5 ml tube, supplemented with 0.1% (w/v) LAP and photo-polymerized under exposure to 405 nm LED device (Droplet Genomics, DG-BR-405) for 20 seconds. Next, microcapsules were rinsed twice in 1×PBS containing 0.1% Pluronic F-68, and then resuspended in 1 ml of 1×IMDM (Gibco, 12440053) or 1×DMEM (Gibco, 61965-059) containing 10% FBS and 1× Penicillin-Streptomycin. Microcapsule suspension was transferred in 60 mm Petri dish, prefilled with 4 ml of culture media and incubated at 37° C./5% CO₂. K-562 cell culture was followed for 4 days and SW620 cell culture was followed for 5 days.

Results presented in FIG. 39 for SW620 cells, and in FIG. 40 for K-562 cells show that both cell types expanded inside the microcapsules and formed individual spheroids (3D cell structures) derived from single-cells.

### Example 21: Some Other Features of the Microcapsules

To further investigate the stability and integrity of the microcapsules we evaluated microcapsule quality under different experimental conditions such as centrifugation force, different buffers, temperature, etc. We first generated water-in-oil droplets using a microfluidics chip 40 μm height and having a nozzle 40 μm wide. The flow-rates used were, 250 μl/hr for 3% (w/v) gelatin methacrylate (Sigma-Aldrich, 900496-1G) dissolved in 1×PBS buffer; 100 μl/h for 15% (w/v) dextran solution in 1×PBS, and 700 μl/hr for the carrier oil.

Droplet generation was performed at room temperature (˜22° C.) and resulting emulsion was collected off-chip into laboratory tube prefilled with 200 μl of light mineral oil. After encapsulation, the emulsion was transferred to 4° C. and incubated for 30-60 minutes to induce solidification (gelation) of the gelatin methacrylate. The intermediate microcapsules were recovered from the emulsion using commercial emulsion breaker (Droplet Genomics, DG-EB-1) and resuspended in ice-cold 1×PBS, supplemented with 0.1% Pluronic F-68. The microcapsule suspension was transferred into a new laboratory tube, supplemented with 0.1% (w/v) LAP and photo-polymerized under exposure to 405 nm LED device (Droplet Genomics, DG-BR-405) for 20 seconds. After photopolymerization, the microcapsules were rinsed twice in 1×PBS buffer containing 0.1% Pluronic F-68, and then applied to different external stress.

FIG. 41 summarizes the results and conditions under which microcapsule stability was evaluated by incubating microcapsules at different buffer for 60 min unless stated otherwise. The said conditions include microcapsules stability evaluation in pure water, 1× Dulbecco's phosphate-buffered saline (DPBS) buffer, 1×DPBS buffer containing 1% Pluronic F68, 1× Hanks' Balanced Salt Solution (HBSS) buffer, 1× saline-sodium citrate (SSC) buffer, 10 mM Tris-HCl, 100 mM NaCl, 5% DMSO in water, 25% glycerol, 70% ethanol, 90% methanol, 90% acetone, 2M Acetic acid for 30 min, 2M NaOH for 15 min. In all conditions tested microcapsules retained their core/shell structure.

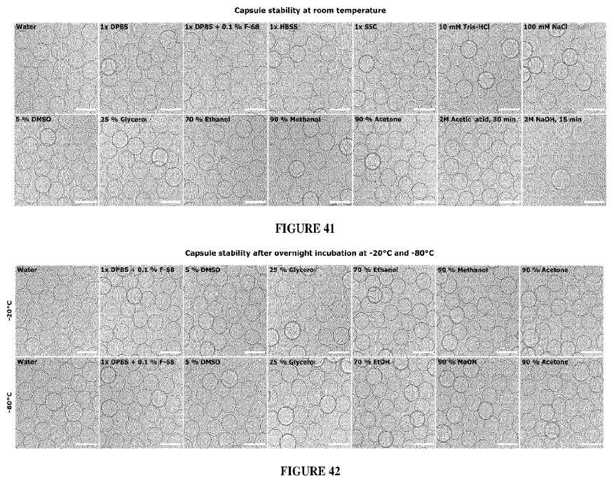

FIG. 42 summarizes the results and conditions under which microcapsule stability was evaluated. The microcapsules were added to a given solution (see list below) and then transferred to either −20° C. or −80° C. and incubated for 14 hours or longer. Following incubation, the microcapsules were centrifuged, supernatant discarded and microcapsules resuspended in 1×PBS supplemented with 0.1% Pluronic F68 and evaluated under bright field microscopy. The solutions in which microcapsules were suspended and cooled down at ˜20° C. or −80° C. included: water, 1×DPBS buffer containing 0.1% Pluronic F68, 5% DMSO, 25% glycerol, 70% ethanol, 90% methanol, 90% acetone. In all conditions tested microcapsules remained retained their core/shell structure.

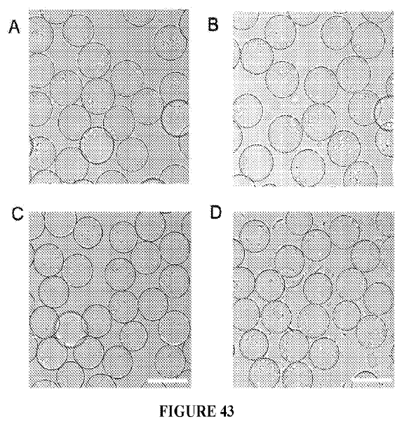

FIG. 43 summarizes other results and conditions under which microcapsule stability was evaluated. In particular the results show that capsules withstand centrifugal force of 20,000 g for 15 min (longer times were not tested). The presented results also show that capsules withstand sonication (1 second=65 joules) using VTUSC3 (Velleman) instrument for at least 15 minutes, (longer times were not tested), but some microcapsules break when sonication is performed for 15 cycles (30 seconds ON and 30 seconds OFF).

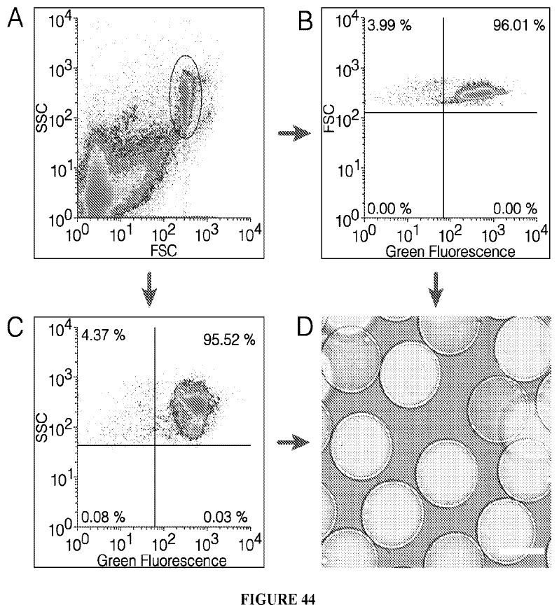

FIG. 44 summarizes results showing that microcapsules of the disclosed invention can be analyzed using Fluorescence-activated cell sorting (FACS) instrument. The microcapsules labelled with a fluorescent dye FITC-dextran 500K were loaded onto Partec CyFlow Space FACS instrument and signal derived from i) forward scatter, ii) side scatter and iii) fluorescence channels was recorded.

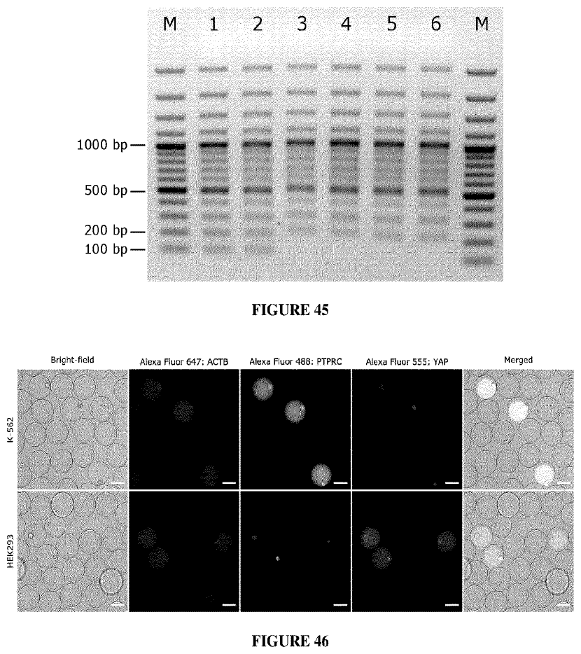

FIG. 45 summarizes the shell permeability measurements. GeneRuler 100 bp Plus DNA ladder (cat no. SM0321, ThermoFisherScientific) was loaded in water-in-oil droplets and biomolecule (DNA) retentions at different stages of microcapsule generation procedure were evaluated. First, the retention of encapsulated DNA fragments was evaluated by breaking droplets immediately after droplet collection off-chip. Next, the retention of encapsulated DNA fragments was evaluated immediately after solidification of the shell and formation of the intermediate-microcapsule. For that purpose, ˜20 μl of intermediate microcapsules were incubated with 0.5 μl Proteinase K (Thermo Fisher Scientific, AM2548) for 10 minutes and following incubation 10 μl of treated sample was combined with 2 μl of 6×DNA Gel Loading Dye (Thermo Fisher Scientific, R0611) and analyzed on 1% agarose gel after DNA electrophoresis. And finally, the DNA fragment retention was evaluated at room or at 50° C. temperature after incubation for 30 min with or without dextranase (the enzyme that reduces the viscosity of the inner core but should not affect the permeability properties of the shell). Results confirmed that microcapsules retain nucleic acid molecules that are 200 bp or longer, the molecular weight of which approximately corresponds to ˜120 kDa. Therefore, the cells within the microcapsule can be contacted with reagents as they diffuse from the external environment of the microcapsule (which may be a reaction buffer in which the microcapsule is suspended) through the semi-permeable shell, into the core. Similarly, reagents and buffer from a previous reaction can be removed from the core by placing the microcapsule in suitable external environment such that the reagents and buffer passively diffuse across the semi-permeable shell into the external environment down a concentration gradient.

### Example 22: Microcapsule Use for Nucleic Acid Analysis

To exemplify the microcapsule applicability for nucleic acid analysis of encapsulated cells we performed RT-PCR assay. The microcapsules comprising either K562 cells or HEK293 cells were generated as described in the Example 10.

Briefly, the K562 and HEK 293 cells were encapsulated separately at a limiting dilution such that each microcapsule, on average, would contain no more than one cell. After cell encapsulation, the microcapsules were washed in 1×PBS buffer containing 0.1% (w/v) Pluronic F-68. After washing the microcapsules were dispersed in ice-cold 70% ethanol and transferred to −20° C. for at least 30-60 min incubation. Upon fixing the encapsulated cells with alcohol, the cell cytoplasm gets dehydrated and biomolecules such as DNA and RNA gets stabilized against the action by nucleases. Fixed cells can be stored at ˜20° C. for extended periods of time before proceeding to rehydration and RT-PCR assay.

After storage at ˜20° C., the tube with microcapsules was equilibrated on ice for 5 minutes. Next, capsules were centrifuged at 2000 g for 2 minutes at 4° C. and washed once with 3×SSC buffer (Invitrogen, 15557044), supplemented with 0.04% BSA, 1 mM DTT and 0.2 U/μl RiboLock RNase Inhibitor (Thermo Scientific, E00381). Next, microcapsules were suspended in 10 mM Tris-HCl [pH 7.5] supplemented with 0.3% (v/v) IGEPAL CA-630, 40 mM DTT and 10 mM EDTA, and incubated at room temperature for 15 minutes, in order to lyse the encapsulated cells. Next, microcapsules were rinsed five-times in 10 mM Tris-HCl [pH 7.5] buffer containing 0.1% (v/v) Triton X-100) and subjected to a reverse transcription (RT) reaction. The cDNA synthesis was performed in 200 μl reaction mix, containing 100 μl close-packed capsules resuspended in 1×RT Buffer (Thermo Scientific, EP0751), 1× Oligo(dT)18 Primer (Thermo Scientific, S0131), 0.5 mM dNTP Mix (Thermo Scientific, R0192), 5 U/μl Maxima H Minus Reverse Transcriptase (Thermo Scientific, EP0751), 0.2 U/μl RiboLock RNase Inhibitor and incubated at 50° C. for 60 minutes. After cDNA synthesis, the microcapsules were rinsed 3-times in washing buffer (10 mM Tris-HCl [pH 7.5] containing 0.1% (v/v) Triton X-100) and then subjected to polymerase chain reaction (PCR).

The PCR was performed in 100 μl reaction volume by mixing 47 μl of closely-packed capsules with 53 μl of PCR reaction mix (Table 3).

TABLE 3
PCR mix composition

| Volume | Material | Final |
|---|---|---|
| 50 μl | 2x Phire Tissue Direst PCR Master Mix | 1x |
| 47 μl | Close-packed capsules | — |
| 0.5 μl | 100 μM 555-YAP-forward | 0.5 μM |
| 0.5 μl | 100 μM YAP-reverse | 0.5 μM |
| 0.5 μl | 100 μM 488- PTPRC -forward | 0.5 μM |
| 0.5 μl | 100 μM PTPRC -forward | 0.5 μM |
| 0.5 μl | 100 μM 647-ACTB-forward | 0.5 μM |
| 0.5 μl | 100 μM ACTB-forward | 0.5 μM |
| 100 μl | Final | — |

During the PCR, the specific markers preferentially expressed in HEK293 cells (YAP marker) or in K562 cells (PTPRC marker) or in both cell lines (ACTB marker), were amplified using marker specific primer set targeting the cDNA of YAP, PTPRC and ACTB transcripts (Table 4).

TABLE 4
The list of multiplex PCR primers used to amplify the markers of interest

| | | | Product |
|---|---|---|---|
| | Name | Sequence (5′-3′) | length |
| SEQ ID 1 | 555-YAP-forward | Alexa Fluor 555- | 550 bp |
| | | CCCTCGTTTTGCCATGAACC | |
| SEQ ID 2 | YAP-reverse | CCAGTGTTCCAAGGTCCACA | |
| SEQ ID 3 | 488- PTPRC -forward | Alexa Fluor 488- | 524 bp |
| | | ACATTGCTGCACAAGGTCCCAG | |
| SEQ ID 4 | PTPRC -reverse | AACCATCAGGCATCTCTGTCGC | |
| SEQ ID 5 | 647-ACTB-forward | Alexa Fluor 647- | 599 bp |
| | | ATTCCTATGTGGGCGACGA | |
| SEQ ID 6 | ACTB-reverse | AATGGTGATGACCTGGCCG | |

Indeed, it should be understood that other markers can be targeted during the PCR step or during RT step. Each primer pair contained a sequence specific oligonucleotide, fluorescently labelled at 5′ end, that served as a forward primer (Table 4). The reverse primer was not labelled with the fluorescent dye. The oligonucleotides targeting different markers carried different fluorescent dyes emitting light at different wavelength, therefore, enabling differentiation of gene expression based on the fluorescence signal. During the PCR, the fluorescently labeled oligonucleotides were incorporated into the PCR amplicons, turning an amplified DNA into a fluorescent product. Although, as exemplified here, only forward primer contained a fluorescent dye, it should be understood to the expert in the field that it should be possible to use both reverse and forward primers labelled with a fluorescent dye and by doing so increase the fluorescence signal intensity of PCR amplicon. The PCR was performed for 40 cycles with Phire Tissue Direct PCR Master Mix (Thermo Scientific, F170L) using thermocycling conditions provided in Table 5.

TABLE 5
The PCR thermocycling conditions

| Step | Temp | Time | | No Cycles |
|---|---|---|---|---|
| Initial denaturation | 98° C. | 5 | min | |
| Amplification | 98° C. | 5 | s | 30 |
| | 64° C. | 5 | s | |
| | 72° C. | 20 | s | |
| Final extension | 72° C. | 1 | min | 1 |

After the PCR, 100 units of Exonuclease I (NEB, M0293L) enzyme was added directly to post-PCR mix, incubated at 37° C. for 15 minutes and rinsed three-times in washing buffer (10 mM Tris-HCl [pH 7.5], 0.1% (v/v) Triton X-100) to remove the excess of fluorescently labeled forward primers that have not been incorporated into PCR amplicons. The capsules then were analyzed using a fluorescence microscopy.

The results presented in FIG. 46 show the fluorescent signal of RT-PCR product distributed within the capsule core. As a result, recording the fluorescence intensity and/or profile of RT-PCR product the expression of gene of interest can be detected and precisely quantified. In a given example, the differential expression of PTPCR and YAP markers in K562 and HEK293 cells can be used to differentiate cells. The capsules harboring K562 cells are positive in PTPCR marker, while capsules harboring HEK293 cell are YAP positive. In addition, both cell types are positive in ACTB marker since this gene is ubiquitously expressed in both cell types. The person with experience in the art will be aware of variety of nucleic acid analysis techniques applicable for analysis of the nucleic acids derived from the encapsulated cells (e.g., PCR, RT, RT-PCR, qPCR, DNA or RNA sequencing, DNA ligation, DNA replication, DNA extension, etc.).

### Example 23: Whole-Genome Amplification

The K-562 cells were encapsulated at a limiting dilution such that each capsule, on average contained no more than one cell. Specifically, K-562 cells were suspended in 15% (w/v) dextran, MW 500k at dilution ˜200k cells/100 μL and co-encapsulated with 3% (w/v) GMA solution in 1×PBS using a microfluidics chip 40 μm height and having a nozzle 40 μm wide. Flow-rates used were: 3% GMA solution—250 μL/h, 15% dextran solution with cells—100 μL/h and the carrier oil—700 μL/h. Encapsulation was performed at room temperature (21-22° C.) for 20-25 minutes. Emulsions were collected in 1.5 mL tube, prefilled with 200 μL of light mineral oil. After encapsulation, emulsions were immediately transferred at 4° C. for 30 minutes to solidify the shell. Continuing procedures on ice, the intermediate-microcapsules were recovered from the emulsion using commercial emulsion breaker (Droplet Genomics, DG-EB-1) and resuspended 1×DPBS buffer supplemented with 0.1% Pluronic F-68. Microcapsule suspension was mixed with 0.1% (w/v) LAP and exposed to 405 nm photo-illumination using LED device (Droplet Genomics, DG-BR-405) for 20 seconds. After cross-linking step, the microcapsules were suspended in GeneJET RNA Purification Kit Lysis Buffer (Thermo Scientific, K0732), containing 40 mM DTT and centrifuged. 900-1000 μL of supernatant was aspirated and replaced with 1 mL of fresh lysis buffer followed by incubation at room temperature for 5 min. After the incubation microcapsules were centrifuged and re-suspended again in the lysis buffer followed by centrifugation. The capsules were rinsed five-times in a washing buffer (10 mM Tris-HCl [pH 7.5] (Invitrogen, 15567027) containing 0.1% (v/v) Triton X-100 (Thermo Scientific, 85111) and subjected to multiple displacement amplification (MDA) reaction. During the washing steps the centrifugation was performed at 1000-2000 g for 2 min.

The MDA was performed in 100 μL reaction volume by mixing 50 μL of closely-packed capsules with 50 μl of MDA reaction mix containing 1× Reaction Buffer (Thermo Scientific, EP0091), 1 mM dNTP Mix (Invitrogen, 18427013), 25 μM Exo-Resistant Random Primer (Thermo Scientific, S0181), 1 mM DTT (Thermo Scientific, R0861) and 0.5 U/μL phi29 DNA Polymerase (Thermo Scientific, EP0091).

Whole genome amplification (WGA) by MDA reaction was performed at 30° C. for 6 hours. After the WGA, the microcapsules were rinsed once in 1 mL of 10 mM Tris-HCl [pH 7.5] buffer containing 0.1% (v/v) Triton X-100 and 5 mM EDTA (Invitrogen, 15575020). Then, capsules were rinsed twice in 10 mM Tris-HCl [pH 7.5] buffer containing 0.1% (v/v) Triton X-100. Post-MDA capsules were stained with 1×SYBR Green I (Invitrogen, S7585) and 5 μM SYTO 9 (Invitrogen, S34854) for 30 minutes at room temperature, then rinsed twice in 10 mM Tris-HCl [pH 7.5] buffer containing 0.1% (v/v) Triton X-100 and analyzed using a fluorescence microscopy.

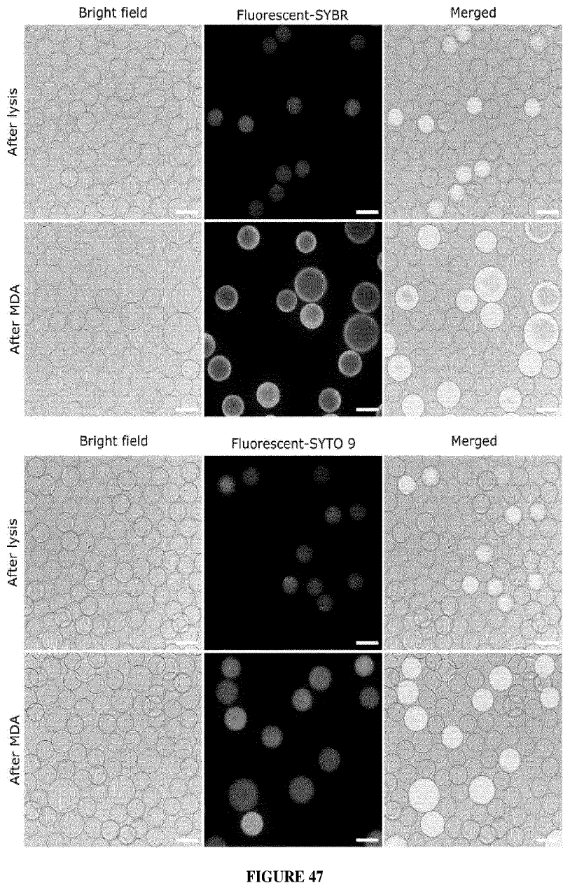

The results presented in FIG. 47 show that individual genomes were successfully amplified in microcapsules comprising cells. Interestingly, upon WGA some capsules swelled and expanded in size, presumably due to increased osmotic pressure exerted on the microcapsule shell.

### Example 24: Long-Term Preservation of Cells in Microcapsules

A long-term storage of mammalian cells and other biological entities often relies on cryopreservation in liquid nitrogen, or at ˜80° C. For example, cells could be cryopreserved for a desirable period of time, transported in a frozen form, and recovered at different location. Biological sample cryopreservation is also important for longitudinal studies, archiving and other applications. To illustrate the microcapsule applicability for encapsulated cell cryopreservation following experiment was conducted.

The microcapsules comprising A549 cells were generated as described in the Example 10 and divided into two equal fractions:

1) The microcapsules in the first fraction were incubated for up to 18 hours in a cell culture in 1×DMEM supplemented with 10% FBS and 1× Penicillin-Streptomycin (PS) in a cell culture incubator at 37° C./5% CO₂, centrifuged, resuspended in 1×DMEM supplemented with 10% FBS, 1×PS and 5% DMSO and frozen in a liquid nitrogen for at least 1 week.

2) The microcapsules in the second fraction were incubated 7 days in a cell culture in 1×DMEM supplemented with 10% FBS and 1×PS in a cell culture incubator at 37° C./5% CO₂, centrifuged, resuspended in 1×DMEM supplemented with 10% FBS, 1×PS and 5% DMSO and frozen in a liquid nitrogen for at least 1 week.

After cryopreservation and storage in a liquid nitrogen tank the microcapsules were thawed, washed 1×DMEM supplemented with 10% FBS and 1×PS and transferred to a cell culture incubator at 37° C./5% CO₂. At selected time points, the cell viability in microcapsules was evaluated using two DNA fluorescent dyes: SYTO 9 (Invitrogen, S34854), which stains nuclei acid in both live and dead cells, and ethidium homodimer-1 (Invitrogen, E1169), which stains nuclei acids in dead cells with compromised membranes.

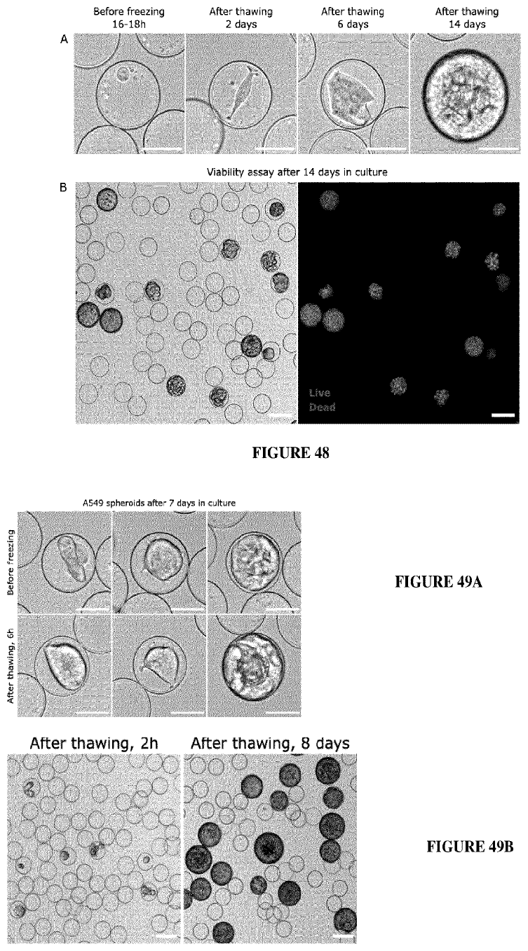

FIG. 48 shows encapsulated cells that were cryopreserved at Day 1 (when the majority of microcapsules on average comprise no more than one cell) stored in a liquid nitrogen for 1 week, recovered and cultivated in cell culture for 14 days. The cell viability (expressed as live cell fraction divided by dead cell fraction and multiplied by 100%) before cryopreservation and after thawing was similar, ˜85% and ˜80%, respectively. The results showed that majority of encapsulated cells (>80%) formed 3D cells structures (spheroids) after 14 days of cell culture in vitro. The fraction of microcapsules having live single-cell before freezing was ˜7.28%; after freezing and thawing it remained almost the same (˜7.16%), and the fraction of microcapsules having a spheroid after 14 days in culture was ˜6.03%.

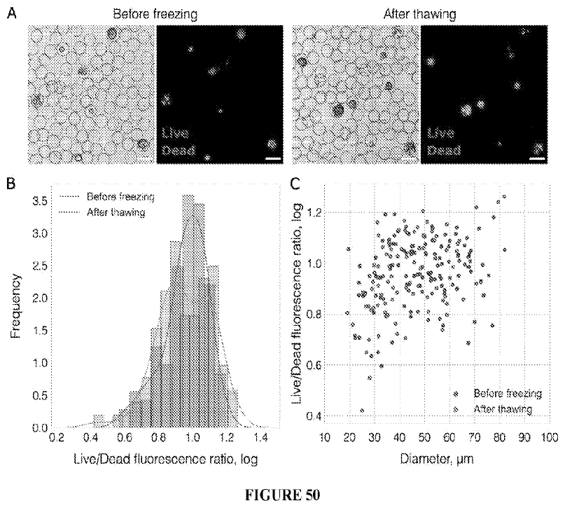

FIG. 49 shows encapsulated cells that were cryopreserved at Day 7 (when the majority of microcapsules on average comprise 3D cell structures/assemblies comprising multiple (>10) cells) stored in a liquid nitrogen for 1 week, recovered and cultivated in cell culture for 7 days.

The results FIGS. 49A and 49B showed that cryopreserved spheroids and those that were not cryopreserved expanded and grew in culture at virtually the same speed, thus suggesting that the cryopreservation of cells in microcapsules may improve their survival during freezing/thawing procedure, preserve 3D cell assemblies, and may help to sustain their biological functions. The results presented in FIG. 50 show that viability of cells in 3D structure, before and after cryopreservation, was nearly the same.

All publications, patents, and patent applications mentioned herein are hereby incorporated by reference in their entirety.

### REFERENCES

1. Chang, T. T. and M. Hughes-Fulford, Monolayer and Spheroid Culture of Human Liver Hepatocellular Carcinoma Cell Line Cells Demonstrate Distinct Global Gene Expression Patterns and Functional Phenotypes. Tissue Engineering Part A, 2009. 15(3): p. 559-567.
2. Tomasi, R. F. X., et al., Individual Control and Quantification of 3D Spheroids in a High-Density Microfluidic Droplet Array. Cell Reports, 2020. 31(8).
3. Xia, Y. and J. C. Izpisua Belmonte, Design Approaches for Generating Organ Constructs. Cell Stem Cell, 2019. 24(6): p. 877-894.
4. Aboulkheyr Es, H., et al., Personalized Cancer Medicine: An Organoid Approach. Trends Biotechnol, 2018. 36(4): p. 358-371.
5. Moshksayan, K., et al., Spheroids-on-a-chip: Recent advances and design considerations in microfluidic platforms for spheroid formation and culture. Sensors and Actuators B-Chemical, 2018. 263: p. 151-176.
6. Kato, Y., et al., Terminal differentiation and calcification in rabbit chondrocyte cultures grown in centrifuge tubes: regulation by transforming growth factor beta and serum factors. Proc Natl Acad Sci USA, 1988. 85(24): p. 9552-6.
7. Ivascu, A. and M. Kubbies, Rapid generation of single-tumor spheroids for high-throughput cell function and toxicity analysis. J Biomol Screen, 2006. 11(8): p. 922-32.
8. Tung, Y. C., et al., High-throughput 3D spheroid culture and drug testing using a 384 hanging drop array. Analyst, 2011. 136(3): p. 473-478.
9. Haisler, W. L., et al., Three-dimensional cell culturing by magnetic levitation. Nature Protocols, 2013. 8(10): p. 1940-1949.
10. Vadivelu, R. K., et al., Microfluidic Technology for the Generation of Cell Spheroids and Their Applications. Micromachines, 2017. 8(4).
11. Lee, J. M., et al., Generation of tumor spheroids using a droplet-based microfluidic device for photothermal therapy. Microsystems & Nanoengineering, 2020. 6(1).
12. Orive, G., et al., History, challenges and perspectives of cell microencapsulation. Trends in Biotechnology, 2004. 22(2): p. 87-92.
13. de Vos, P., et al., Polymers in cell encapsulation from an enveloped cell perspective. Advanced Drug Delivery Reviews, 2014. 67-68: p. 15-34.
14. Sabhachandani, P., et al., Generation and functional assessment of 3D multicellular spheroids in droplet based microfluidics platform. Lab on a Chip, 2016. 16(3): p. 497-505.
15. Sart, S., et al., Multiscale cytometry and regulation of 3D cell cultures on a chip. Nature Communications, 2017. 8.
16. Hong, S. M., et al., Collagen microsphere production on a chip. Lab on a Chip, 2012. 12(18): p. 3277-3280.
17. Cha, C. E. Y., et al., Microfluidics-Assisted Fabrication of Gelatin-Silica Core-Shell Microgels for Injectable Tissue Constructs. Biomacromolecules, 2014. 15(1): p. 283-290.
18. Li, W., et al., Microfluidic fabrication of microparticles for biomedical applications. Chemical Society Reviews, 2018. 47(15): p. 5646-5683.
19. Yu, L. F., et al., Alginate core-shell beads for simplified three-dimensional tumor spheroid culture and drug screening. Biomedical Microdevices, 2015. 17(2).
20. Siltanen, C., et al., Microfluidic fabrication of bioactive microgels for rapid formation and enhanced differentiation of stem cell spheroids. Acta Biomaterialia, 2016. 34: p. 125-132.
21. Ma, M. L., et al., Core-Shell Hydrogel Microcapsules for Improved Islets Encapsulation. Advanced Healthcare Materials, 2013. 2(5): p. 667-672.
22. Kim, C., et al., Generation of core-shell microcapsules with three-dimensional focusing device for efficient formation of cell spheroid. Lab on a Chip, 2011. 11(2): p. 246-252.
23. Alessandri, K., et al., Cellular capsules as a tool for multicellular spheroid production and for investigating the mechanics of tumor progression in vitro. Proceedings of the National Academy of Sciences of the United States of America, 2013. 110(37): p. 14843-14848.
24. Jo, Y. K. and D. Lee, Biopolymer Microparticles Prepared by Microfluidics for Biomedical Applications. Small, 2020. 16(9).
25. Huang, H. S., et al., Generation and manipulation of hydrogel microcapsules by droplet-based microfluidics for mammalian cell culture. Lab on a Chip, 2017. 17(11): p. 1913-1932.
26. Sakai, S., et al., Enzymatically fabricated and degradable microcapsules for production of multicellular spheroids with well-defined diameters of less than 150 mu m. Biomaterials, 2009. 30(30): p. 5937-5942.
27. Correia, C. R., et al., Liquified chitosan-alginate multilayer capsules incorporating poly(L-lactic acid) microparticles as cell carriers. Soft Matter, 2013. 9(7): p. 2125-2130.
28. Fattahi, P., et al., Core-shell hydrogel microcapsules enable formation of human pluripotent stem cell spheroids and their cultivation in a stirred bioreactor. Scientific Reports, 2021. 11(1).
29. Hwang, C. M., et al., Fabrication of three-dimensional porous cell-laden hydrogel for tissue engineering. Biofabrication, 2010. 2(3).
30. Vijayakumar, K., et al., Rapid cell extraction in aqueous two-phase microdroplet systems. Chemical Science, 2010. 1(4): p. 447-452.
31. Leonaviciene, G., et al., Multi-step processing of single cells using semi-permeable capsules. Lab on a Chip, 2020. 20(21): p. 4052-4062.
32. Dinh, N. D., et al., Functional reservoir microcapsules generated via microfluidic fabrication for long-term cardiovascular therapeutics. Lab Chip, 2020. 20(15): p. 2756-2764.
33. Watanabe, T., I. Motohiro, and T. Ono, Microfluidic Formation of Hydrogel Microcapsules with a Single Aqueous Core by Spontaneous Cross-Linking in Aqueous Two-Phase System Droplets. Langmuir, 2019. 35(6): p. 2358-2367.
34. Antona, S., I. Platzman, and J. P. Spatz, Droplet-Based Cytotoxicity Assay: Implementation of Time-Efficient Screening of Antitumor Activity of Natural Killer Cells. Acs Omega, 2020. 5(38): p. 24674-24683.
35. Erman Salih Istifli, M. T. H.a.H. B. I., Cell Division, Cytotoxicity, and the Assays Used in the Detection of Cytotoxicity. 2019: IntechOpen.
36. Shembekar, N., et al., Single-Cell Droplet Microfluidic Screening for Antibodies Specifically Binding to Target Cells. Cell Reports, 2018. 22(8): p. 2206-2215.
37. Segaliny, A. I., et al., Functional TCR T cell screening using single-cell droplet microfluidics. Lab on a Chip, 2018. 18(24): p. 3733-3749.
38. Antona, S., et al., Droplet-Based Combinatorial Assay for Cell Cytotoxicity and Cytokine Release Evaluation. Advanced Functional Materials, 2020. 30(46).
39. Lee, K. Y. and D. J. Mooney, Alginate: Properties and biomedical applications. Progress in Polymer Science, 2012. 37(1): p. 106-126.
40. Kikuchi, A., et al., Effect of Ca2+-alginate gel dissolution on release of dextran with different molecular weights. Journal of Controlled Release, 1999. 58(1): p. 21-28.
41. Hess, M., et al., Terminology of polymers containing ionizable or ionic groups and of polymers containing ions (IUPAC Recommendations 2006). Pure and Applied Chemistry, 2006. 78(11): p. 2067-2074.
42. Sun, T. L., et al., Physical hydrogels composed of polyampholytes demonstrate high toughness and viscoelasticity. Nature Materials, 2013. 12(10): p. 932-937.
43. Haag, S. and M. Bernards, Polyampholyte Hydrogels in Biomedical Applications. Gels, 2017. 3(4).
44. Brangwynne, C. P., P. Tompa, and R. V. Pappu, Polymer physics of intracellular phase transitions. Nature Physics, 2015. 11(11): p. 899-904.
45. McCarty, J., et al., Complete Phase Diagram for Liquid-Liquid Phase Separation of Intrinsically Disordered Proteins. The Journal of Physical Chemistry Letters, 2019. 10(8): p. 1644-1652.
46. Schuster, B. S., et al., Controllable protein phase separation and modular recruitment to form responsive membraneless organelles. Nature Communications, 2018. 9(1).
47. Quiroz, F. G. and A. Chilkoti, Sequence heuristics to encode phase behaviour in intrinsically disordered protein polymers. Nature Materials, 2015. 14(11): p. 1164-1171.
48. Muiznieks, L. D. and F. W. Keeley, Proline periodicity modulates the self-assembly properties of elastin-like polypeptides. J Biol Chem, 2010. 285(51): p. 39779-89.
49. Simon, J. R., et al., Programming molecular self-assembly of intrinsically disordered proteins containing sequences of low complexity. Nature Chemistry, 2017. 9(6): p. 509-515.
50. Uversky, V. N., et al., Intrinsically disordered proteins as crucial constituents of cellular aqueous two phase systems and coacervates. FEBS Letters, 2015. 589(1): p. 15-22.
51. Dzuricky, M., S. Roberts, and A. Chilkoti, Convergence of Artificial Protein Polymers and Intrinsically Disordered Proteins. Biochemistry, 2018. 57(17): p. 2405-2414.
52. Danielsen, S. P. O., et al., Molecular design of self-coacervation phenomena in block polyampholytes. Proceedings of the National Academy of Sciences, 2019. 116(17): p. 8224-8232.
53. Thorsen, T., et al., Dynamic pattern formation in a vesicle-generating microfluidic device. Phys Rev Lett, 2001. 86(18): p. 4163-6.
54. Garstecki, P., et al., Formation of monodisperse bubbles in a microfluidic flow-focusing device. Applied Physics Letters, 2004. 85(13): p. 2649-2651.
55. Sugiura, S., M. Nakajima, and M. Seki, Prediction of Droplet Diameter for Microchannel Emulsification. Langmuir, 2002. 18(10): p. 3854-3859.
56. Ward, T., et al., Microfluidic flow focusing: Drop size and scaling in pressure versus flow-rate-driven pumping. Electrophoresis, 2005. 26(19): p. 3716-3724.
57. Anna, S. L., N. Bontoux, and H. A. Stone, Formation of dispersions using “flow focusing” in microchannels. Applied Physics Letters, 2003. 82(3): p. 364-366.
58. Nie, Z., et al., Emulsification in a microfluidic flow-focusing device: effect of the viscosities of the liquids. Microfluidics and Nanofluidics, 2008. 5(5): p. 585-594.
59. Zheng, B., J. D. Tice, and R. F. Ismagilov, Formation of Droplets of Alternating Composition in Microfluidic Channels and Applications to Indexing of Concentrations in Droplet-Based Assays. Analytical Chemistry, 2004. 76(17): p. 4977-4982.
60. Teh, S.-Y., et al., Droplet microfluidics. Lab on a Chip, 2008. 8(2).
61. Chao, Y. and H. C. Shum, Emerging aqueous two-phase systems: from fundamentals of interfaces to biomedical applications. Chemical Society Reviews, 2020. 49(1): p. 114-142.
62. Holtze, C., et al., Biocompatible surfactants for water-in-fluorocarbon emulsions. Lab on a Chip, 2008. 8(10).
63. Chao, Y. and H. C. Shum, Emerging aqueous two-phase systems: from fundamentals of interfaces to biomedical applications. Chem Soc Rev, 2020. 49(1): p. 114-142.
64. Ananthapadmanabhan, K. P. and E. D. Goddard, Aqueous biphase formation in polyethylene oxide-inorganic salt systems. Langmuir, 2002. 3(1): p. 25-31.
65. Elbert, D. L., Liquid-liquid two-phase systems for the production of porous hydrogels and hydrogel microspheres for biomedical applications: A tutorial review. Acta Biomaterialia, 2011. 7(1): p. 31-56.
66. Comert, F. and P. L. Dubin, Liquid-liquid and liquid-solid phase separation in protein-polyelectrolyte systems. Advances in Colloid and Interface Science, 2017. 239: p. 213-217.
67. Urry, D. W., B. Starcher, and S. M. Partridge, Coacervation of Solubilized Elastin effects a Notable Conformational Change. Nature, 1969. 222(5195): p. 795-796.
68. Overbeek, J. T. and M. J. Voorn, Phase separation in polyelectrolyte solutions; theory of complex coacervation. J Cell Physiol Suppl, 1957. 49(Suppl 1): p. 7-22; discussion, 22-6.
69. Sing, C. E. and S. L. Perry, Recent progress in the science of complex coacervation. Soft Matter, 2020. 16(12): p. 2885-2914.
70. Mountain, G. A. and C. D. Keating, Formation of Multiphase Complex Coacervates and Partitioning of Biomolecules within them. Biomacromolecules, 2019. 21(2): p. 630-640.
71. Nakashima, K. K., M. A. Vibhute, and E. Spruijt, Biomolecular Chemistry in Liquid Phase Separated Compartments. Frontiers in Molecular Biosciences, 2019. 6.
72. Wang, X., P. Zhang, and L. Tian, Spatiotemporal organization of coacervate microdroplets. Current Opinion in Colloid & Interface Science, 2021. 52.
73. Andr6, A. A. M. and E. Spruijt, Liquid-Liquid Phase Separation in Crowded Environments. International Journal of Molecular Sciences, 2020. 21(16).
74. Chen, Y., et al., Construction of coacervate-in-coacervate multi-compartment protocells for spatial organization of enzymatic reactions. Chemical Science, 2020. 11(32): p. 8617-8625.
75. Dubin, P., et al., Macromolecular Complexes in Chemistry and Biology. 1994.
76. Roberts, S., et al., Complex microparticle architectures from stimuli-responsive intrinsically disordered proteins. Nature Communications, 2020. 11(1).
77. Van Den Bulcke, A. I., et al., Structural and rheological properties of methacrylamide modified gelatin hydrogels. Biomacromolecules, 2000. 1(1): p. 31-8.
78. Sakai, S., et al., An injectable, in situ enzymatically gellable, gelatin derivative for drug delivery and tissue engineering. Biomaterials, 2009. 30(20): p. 3371-3377.
79. Li, F. Y., et al., Microfluidic Encapsulation of Human Mesenchymal Stem Cells for Articular Cartilage Tissue Regeneration. Acs Applied Materials & Interfaces, 2017. 9(10): p. 8589-8601.
80. Babin, H. and E. Dickinson, Influence of transglutaminase treatment on the thermoreversible gelation of gelatin. Food Hydrocolloids, 2001. 15(3): p. 271-276.
81. Aisenbrey, E. A. and W. L. Murphy, Synthetic alternatives to Matrigel. Nature Reviews Materials, 2020. 5(7): p. 539-551.
82. Mazutis, L., et al., Single-cell analysis and sorting using droplet-based microfluidics. Nat Protoc, 2013. 8(5): p. 870-91.
83. Bounab, Y., et al., Dynamic single-cell phenotyping of immune cells using the microfluidic platform DropMap. Nature Protocols, 2020. 15(9): p. 2920-2955.
84. Wang, L., et al., Porous and responsive hydrogels for cell therapy. Current Opinion in Colloid & Interface Science, 2018. 38: p. 135-157.
85. Sepantafar, M., et al., Engineered Hydrogels in Cancer Therapy and Diagnosis. Trends in Biotechnology, 2017. 35(11): p. 1074-1087.
86. Foyt, D. A., et al., Exploiting Advanced Hydrogel Technologies to Address Key Challenges in Regenerative Medicine. Advanced Healthcare Materials, 2018. 7(8).
87. Link, D. R., et al., Geometrically mediated breakup of drops in microfluidic devices. Phys Rev Lett, 2004. 92(5): p. 054503.

1.-86. (canceled)

87. A microcapsule comprising:
(a) a core comprising a polyhydroxy compound and/or an antichaotropic agent; and
(b) a semi-permeable shell concentrically surrounding the core;
wherein the semi-permeable shell comprises a gel formed from a polyampholyte, wherein the polyampholyte in the gel is covalently cross-linked, wherein the microcapsule further comprises at least one biological entity.

88. The microcapsule of claim 87, wherein the at least one biological entity is selected from a cell, a microorganism, a bacterium, a virus, or a nucleic acid.

89. The microcapsule of claim 88, wherein the at least one cell is at least one prokaryotic cell, such as a bacterial cell or an archaea cell, or at least one eukaryotic cell.

90. The microcapsule of claim 87, wherein the microcapsule comprises at least one biological entity and the at least one biological entity is at least one nucleic acid, optionally wherein the at least one nucleic acid is at least 100 nucleotides long.

91. The microcapsule of claim 87, wherein the microcapsule comprises at least one solid particle, optionally wherein the at least one solid particle is a metal nanoparticle, a mineral particle, a polymer particle, a fluorescent nanoparticle, a magnetic nanoparticle or a composite particle.

92. The microcapsule of claim 87, wherein the polyampholyte is a single type of biopolymer, such as polypeptide, or is a mixture of two or more polyampholytes or a mixture of at least one polyampholyte and at least one of polyelectrolyte, polysaccharide, oligosaccharide, sugar, or synthetic polymer, such as poly(ethylene glycol), or is a thermo-responsive polymer capable of forming a thermo-reversible gel in response to a temperature change.

93. The microcapsule of claim 87, wherein the polyampholyte is:
(a) is a biopolymer, a modified biopolymer or a synthetic polymer, wherein the modified biopolymer is the polyampholyte modified with one or more chemically cross-linkable groups for the covalent cross-linking, wherein the polyampholyte has a degree of substitution of 10 to 90%, preferably 40 to 90%, more preferably 60 to 80%.
(b) comprises peptide bonds, and/or
(c) is a peptide, a polypeptide, an oligopeptide or a protein.

94. The microcapsule of claim 87, wherein the semi-permeable shell comprises peptide bonds, and which are biodegradable with a protease to release the inner content of the microcapsule.

95. The microcapsule of claim 94, wherein the polyampholyte comprises amino acids, and where in at least 10% of amino acids in the polyampholyte are disorder-promoting amino acids, preferably wherein at least 30% of amino acids in the polyampholyte are disorder-promoting amino acids, where the disorder promoting amino acids are selected from proline, glycine, glutamic acid/glutamate, serine, lysine, alanine, arginine, and glutamine.

96. The microcapsule of claim 87, wherein the polyampholyte is selected from the group consisting of collagen, mucin, laminin, elastin, elastin-like polypeptides, fibrin, silk fibrion, fibronectin, vimentin, glycinin, gluten, casein, or hydrolyzed forms thereof, such as gelatin.

97. The microcapsule of claim 87, wherein the gel is formed from a polyampholyte modified with a chemical group, selected from the group consisting of acrydite, acrylate, methacryloyl, acrylamide, methacrylamide, bisacrylamide, methacrylate, methacrylic acid, acrylic acid, polyacrylic acid, methacrylic anhydride, acryloyl, vinyl, vinylsulfone, vinylpyrrolidone, thiol, disulphide, cystamine, carboxyl, amine, imine, azide, triazole, tetrazine, azidophenylalanine, alkynyl, alkenyl, alkynes, diisocyanate, hydroxypropionic acid, hydroxy phenol, azobenzene, methylcyclopropene, trans-cyclooctene (TCO), norbornene, diacrylcyclooctyne (DBCO) or cyclooctanyl moieties and/or reagents.

98. The microcapsule of claim 87, wherein the microcapsule comprise at least one biological entity in the core and the shell is permeable to a reagent, an enzyme, a nutrient, and/or a substrate but not permeable to the biological entity of interest.

99. The microcapsule of claim 87, wherein the semi-permeable shell is permeable to smaller molecular weight compounds having a molecular weight of 200,000±100,000 Da or less through the shell, while mostly impermeable to larger molecular weight compounds, preferably wherein the semi-permeable shell allows for diffusion of smaller molecular weight compounds of having a molecular weight of 120,000±60,000 Da or less through the shell, while retaining larger molecular weight compounds.

100. The microcapsule of claim 87, wherein the core is a liquid, a semi-liquid, a polymer, or a hydrogel and comprises the polyhydroxy biodegradable compound selected from a polyelectrolyte, polysaccharide, a carbohydrate, an oligosaccharide, or a sugar, which can be natural or synthetic.

101. The microcapsule of claim 87, wherein the polyhydroxy compound has a molecular weight of 300 Da to 5000 kDa, preferably 10 kDa to 800 kDa, and even more preferably 400 kDa to 600 kDa.

102. The microcapsule of claim 87, wherein the polyhydroxy compound can be hydrolyzed upon treatment with a hydrolase enzyme (e.g., a glycosidase, a dextranase, an amylase, a sucrase, or a cellulase) to reduce the viscosity of the core.

103. The microcapsule of claim 87, wherein the antichaotropic agent is a kosmotropic salt such as a sulphate, a phosphate or a citrate, preferably ammonium sulphate.

104. The microcapsule of claim 87, wherein the microcapsule is from 1 μm to 100,000 μm in diameter, preferably between 1 μm and 500 μm in diameter, and more preferably between 20 μm and 200 μm in diameter and the shell of the microcapsule is from 0.2 to 100 μm thick, preferably from 1 to 10 μm thick.

105. The microcapsule of claim 87, wherein the microcapsule does not disintegrate during PCR, thermocycling or an incubation at an elevated temperature, such as at least 10 minutes at 50±10° C. or even at 90±10° C. and retains encapsulate biological species of interest during cryopreservation.

106. The microcapsule of claim 87, wherein the microcapsule is formed by (a) changing the temperature of a droplet, wherein the droplet is a water-in-oil droplet, a water-in-water droplet or a water-in-air droplet, comprising a core enriched in the polyhydroxy compound and/or antichaotropic agent and a shell enriched in the polyampholyte, to induce gelation of the polyampholyte; and (b) and covalently cross-linking the gelled polyampholyte

107. A composition comprising one or more microcapsule of claim 87 in a carrier oil, or in an aqueous solution.

108. The composition of claim 107, wherein the aqueous solution is a cell culture medium, or a cell storage medium or an aqueous buffer, optionally wherein the aqueous buffer is a buffer for washing cells or lysing the encapsulated cells.

109. An in vitro method for cultivating at least one cell encapsulated in or attached to an inner surface or an outer surface of a microcapsule according to claim 87, comprising incubating the microcapsule in an aqueous environment suitable to allow for cell survival, cell growth and/or cell proliferation.

110. The in vitro method of claim 109, wherein the aqueous environment is a suitable growth media comprising one or more nutrients needed for encapsulated cell survival and/or growth, wherein where the cell is encapsulated the method comprising allowing the one or more nutrients to diffuse into the core of the capsule.

111. The in vitro method of claim 109, wherein the method comprises releasing the encapsulated cells from the microcapsule after cultivation by hydrolyzing the microcapsule.

112. A method of storing cells, comprising suspending the microcapsule according to claim 88, in a cell storage medium comprising a cryoprotectant, and storing the microcapsule at temperature of sub-zero degrees Celsius,

113. The method of claim 112, wherein the at least one cell is comprised in the core of the microcapsule, optionally wherein the at least one cell in the core is at an inner surface of the semi-permeable shell, further optionally wherein the at least one cell is attached to the inner surface of the semi-permeable shell.

114. A method of releasing the contents of the core of a microcapsule of claim 87, the method comprising breaking the semi-permeable shell of the microcapsule.

115. A method of delivering at least one biological entity to a subject for the treatment of a disease, a disorder or an injury in the subject, the method comprising administering a microcapsule of claim 87, to the subject, wherein the microcapsule comprises the at least one biological entity, optionally wherein the at least one biological entity is comprised in the core of the microcapsule.

116. A microcapsule of claim 87 for use in delivering a medical therapy, wherein the microcapsule comprises at least one biological entity for the medical therapy, optionally wherein the at least one biological entity is comprised in the core of the microcapsule.

117. A method for the manufacture of a microcapsule medicament for delivering a treatment to a subject, wherein the microcapsule of claim 87 comprises at least one biological entity for the treatment.


SEQUENCE LISTING (6 sequences)

SEQ ID NO: 1 - protein, 20 aa, unidentified
CCCTCGTTTTGCCATGAACC

SEQ ID NO: 2 - protein, 20 aa, unidentified
CCAGTGTTCCAAGGTCCACA

SEQ ID NO: 3 - protein, 22 aa, unidentified
ACATTGCTGCACAAGGTCCCAG

SEQ ID NO: 4 - protein, 22 aa, unidentified
AACCATCAGGCATCTCTGTCGC

SEQ ID NO: 5 - protein, 19 aa, unidentified
ATTCCTATGTGGGCGACGA

SEQ ID NO: 6 - protein, 19 aa, unidentified
AATGGTGATGACCTGGCCG